SEQUENCE LISTING (65 sequences)

SEQ ID NO: 1 - protein, 4 aa, Artificial Sequence
  Substrate
GKKR

SEQ ID NO: 2 - protein, 4 aa, Artificial Sequence
  Substrate
RRKK

SEQ ID NO: 3 - protein, 8 aa, Artificial Sequence
  Substrate
RLIQKRSD

SEQ ID NO: 4 - protein, 8 aa, Artificial Sequence
  Substrate
LIQKRSDV

SEQ ID NO: 5 - protein, 6 aa, Artificial Sequence
  Substrate
FVNQHL

SEQ ID NO: 6 - protein, 8 aa, Artificial Sequence
  Substrate
LVEALYLV

SEQ ID NO: 7 - protein, 8 aa, Artificial Sequence
  Substrate
EALYLVCG

SEQ ID NO: 8 - protein, 8 aa, Artificial Sequence
  Substrate
ERGFFYTP

SEQ ID NO: 9 - protein, 7 aa, Artificial Sequence
  Substrate
PALFFRL

SEQ ID NO: 10 - protein, 5 aa, Artificial Sequence
  Substrate
LVIHT

SEQ ID NO: 11 - protein, 8 aa, Artificial Sequence
  Substrate
HQVYFVRK

SEQ ID NO: 12 - protein, 7 aa, Artificial Sequence
  Substrate
PARFFRL

SEQ ID NO: 13 - protein, 7 aa, Artificial Sequence
  Substrate
PAEFFAL

SEQ ID NO: 14 - protein, 7 aa, Artificial Sequence
  Substrate
PLEMFAL

SEQ ID NO: 15 - protein, 6 aa, Artificial Sequence
  Substrate
HSSKLQ

SEQ ID NO: 16 - protein, 6 aa, Artificial Sequence
  Substrate
QFYSSN

SEQ ID NO: 17 - protein, 14 aa, Artificial Sequence
  Substrate
GAGLRLSSYYSGAG

SEQ ID NO: 18 - protein, 10 aa, Artificial Sequence
  Substrate
SSIYSQTEEQ

SEQ ID NO: 19 - protein, 8 aa, Artificial Sequence
  Substrate
GVVNASCR

SEQ ID NO: 20 - protein, 8 aa, Artificial Sequence
  Substrate
SYVLASVS

SEQ ID NO: 21 - protein, 8 aa, Artificial Sequence
  Substrate
NNVEAATS

SEQ ID NO: 22 - protein, 8 aa, Artificial Sequence
  Substrate
TAVNASGN

SEQ ID NO: 23 - protein, 4 aa, Artificial Sequence
  Substrate
VXAX

SEQ ID NO: 24 - protein, 5 aa, Artificial Sequence
  Substrate
VVXAS

SEQ ID NO: 25 - protein, 7 aa, Artificial Sequence
  Substrate
VVNASCR

SEQ ID NO: 26 - protein, 11 aa, Artificial Sequence
  Substrate
RGVVNASSRLA

SEQ ID NO: 27 - protein, 12 aa, Artificial Sequence
  Substrate
RGVVNASSRLAC

SEQ ID NO: 28 - protein, 7 aa, Artificial Sequence
  Substrate
XXXXPXX

SEQ ID NO: 29 - protein, 7 aa, Artificial Sequence
  Substrate
XXXXVXQ

SEQ ID NO: 30 - protein, 10 aa, Artificial Sequence
  Substrate
KVKVXVVQPK

SEQ ID NO: 31 - protein, 7 aa, Artificial Sequence
  Substrate
PPXXLPI

SEQ ID NO: 32 - protein, 11 aa, Artificial Sequence
  Substrate
LEALFQGPDSQ

SEQ ID NO: 33 - protein, 11 aa, Artificial Sequence
  Substrate
LEALFQGPPVY

SEQ ID NO: 34 - protein, 11 aa, Artificial Sequence
  Substrate
LEALFQGPPIY

SEQ ID NO: 35 - protein, 11 aa, Artificial Sequence
  Substrate
LEALFQGPPAY

SEQ ID NO: 36 - protein, 11 aa, Artificial Sequence
  Substrate
LEALFQGPPQF

SEQ ID NO: 37 - protein, 11 aa, Artificial Sequence
  Substrate
LEALFQGPPTF

SEQ ID NO: 38 - protein, 11 aa, Artificial Sequence
  Substrate
LEALFQGPIEF

SEQ ID NO: 39 - protein, 11 aa, Artificial Sequence
  Substrate
MEALFQGPLQY

SEQ ID NO: 40 - protein, 11 aa, Artificial Sequence
  Substrate
MEALFQGPIQY

SEQ ID NO: 41 - protein, 11 aa, Artificial Sequence
  Substrate
MEALFQGPLEF

SEQ ID NO: 42 - protein, 11 aa, Artificial Sequence
  Substrate
MEALFQGPLRY

SEQ ID NO: 43 - protein, 11 aa, Artificial Sequence
  Substrate
IEALFQGPPVF

SEQ ID NO: 44 - protein, 11 aa, Artificial Sequence
  Substrate
IEALFQGPPQF

SEQ ID NO: 45 - protein, 11 aa, Artificial Sequence
  Substrate
IEALFQGPPKF

SEQ ID NO: 46 - protein, 11 aa, Artificial Sequence
  Substrate
IEALFQGPLKF

SEQ ID NO: 47 - protein, 116 aa, Homo sapiens
APFRSALESSPADPATLSEDEARLLLAALVQDYVQMKASELEQEQEREGSSLDSPRSKRCGNLSTCMLGTYTQDFNKFHTFPQTAIGVGAPGKKRDMSSDLERDHRPHVSMPQNAN

SEQ ID NO: 48 - protein, 7 aa, Artificial Sequence
  Herpes simplex virus protease substrate consensus
XXXASXX

SEQ ID NO: 49 - protein, 7 aa, Artificial Sequence
  West Nile virus protease substrate consensus
XXXXXXX

SEQ ID NO: 50 - protein, 9 aa, Artificial sequence
  West Nile virus protease substrate consensus
PNRKRGWPA

SEQ ID NO: 51 - protein, 11 aa, Artificial sequence
  West Nile virus protease substrate consensus
FASGKRSQIGV

SEQ ID NO: 52 - protein, 4 aa, Artificial sequence
  Enterovirus 3C protease substrate consensus
FQGP

SEQ ID NO: 53 - protein, 8 aa, Artificial sequence
  Coxsackievirus protease substrate consensus
TTGXXXQQ

SEQ ID NO: 54 - protein, 6 aa, Artificial sequence
  Coxsackievirus protease substrate consensus
AXGXQX

SEQ ID NO: 55 - protein, 8 aa, Artificial sequence
  Echovirus protease substrate consensus
XTGXXXQQ

SEQ ID NO: 56 - protein, 8 aa, Artificial sequence
  Echovirus protease substrate consensus
XXGAFXQQ

SEQ ID NO: 57 - protein, 6 aa, Artificial sequence
  Echovirus protease substrate consensus
XFGQQX

SEQ ID NO: 58 - protein, 9 aa, Artificial sequence
  Echovirus protease substrate consensus
XXGXFXQQS

SEQ ID NO: 59 - protein, 12 aa, Artificial Sequence
  Substrate
LEALFQGPPVYC

SEQ ID NO: 60 - DNA, 22 bp, Artificial Sequence
  Primer
caccatgacgatggacgagcag

SEQ ID NO: 61 - DNA, 4 bp, Artificial Sequence
  Primer extension
cacc

SEQ ID NO: 62 - DNA, 22 bp, Artificial Sequence
  Primer
tcacgccttgacgtatgactcg

SEQ ID NO: 63 - protein, 11 aa, Artificial Sequence
  Substrate
LEALFQGPDSQ

SEQ ID NO: 64 - protein, 14 aa, Artificial Sequence
  Substrate
KLEALFQGPPVYEA

SEQ ID NO: 65 - protein, 12 aa, Artificial Sequence
  Substrate
TSAVLQSGFRDK

(19) United States
(12) Patent Application Publication
(10) Pub. No.: US 2011/0306035 A1
(43) Pub. Date: Dec. 15, 2011

(54) Methods and Compositions for Detection of a Pathogen, Disease, Medical Condition, or Biomarker Thereof

(75) Inventors: Dorit Arad, Tel Aviv (IL); Yaniv Nevo, Ness-Ziona (IL); Assaf Ezra, Ness-Ziona (IL)

(73) Assignee: Dorit Arad, Tel Aviv (IL)

(21) Appl. No.: 13/056,486

(22) PCT Filed: Jul. 28, 2009
(86) PCT No.: PCT/IB09/06643

§ 371 Date: Apr. 18, 2011

Related U.S. Application Data
(60) Provisional application No. 61/129,887, filed on Jul. 28, 2008.
(60) Provisional application No. 61/136,105, filed on Aug. 12, 2008.
(60) Provisional application No. 61/136,143, filed on Aug. 14, 2008.
(60) Provisional application No. 61/159,485, filed on Mar. 12, 2009.

(51) Int. Cl. C12Q 1/37; C12Q 1/04; C12Q 1/34; C12Q 1/70

(52) U.S. Cl. 435/5; 435/34; 435/23; 435/18

(57) ABSTRACT

Provided are methods for detecting the presence or absence of a pathogen, disease, or medical condition, or biomarker thereof, using an enzymatic activity assay. In one embodiment, the method provided utilizes competitive inhibition of an enzyme for detecting a pathogen, disease, or medical condition, or biomarker thereof, in a subject. The method comprises providing a biological sample from the subject that may or may not contain an endogenous substrate. A test reaction is provided by contacting the biological sample with an enzyme indicative of the biomarker of a pathogen, disease, or medical condition and a substrate comprising a signaling moiety. The enzyme modifies the endogenous substrate and the substrate comprising the signaling moiety. Modification of the substrate comprising the signaling moiety by the enzyme produces a signal from the signaling moiety. Data from a control reaction comprising the enzyme and the substrate comprising the signaling moiety is further provided. The signal produced by the signaling moiety in the test reaction is detected. The presence of the biomarker of the pathogen, disease, or medical condition is indicated by a difference caused by the presence of the endogenous substrate in the biological sample between the signal produced in the test reaction and the data from the control reaction. In another embodiment, there is provide a method of detecting the presence or absence of enzymatic activity in a biological sample indicative of a pathogen, disease, or medical condition, or biomarker thereof, in a subject. The method comprises contacting a biological sample obtained from a subject that may or may not contain an enzyme with a substrate of the enzyme to be detected. The substrate comprises a signaling molecule such that when the enzyme is present in the biological sample, the enzyme modifies the substrate and the signaling moiety emits a signal, indicating the presence of a pathogen, disease, or a medical condition, or a biomarker thereof, in the subject.

This application claims the benefit of U.S. Provisional Application No. 61/129,887, filed Jul. 28, 2008; U.S. Provisional Application No. 61/136,105, filed Aug. 12, 2008; U.S. Provisional Application No. 61/136,143, filed Aug. 14, 2008; and U.S. Provisional Application No. 61/159,485, filed Mar. 12, 2009.

Provided are methods for detecting a pathogen or a disease or a medical condition using an enzymatic activity assay.

Direct detection of the levels of a pathogen, disease or medical condition, or biomarkers that indicate the presence of a pathogen, disease, or a medical condition (hereinafter “biomarkers thereof”), in a subject can be achieved using immunoassays or nucleic acid amplification methods, such as PCR. There is, however, a continuing need for a rapid sensitive method for detecting a pathogen, or a disease or a medical condition, or a biomarker thereof, in a subject.

Many organisms have characteristic enzymatic activity associated with different stages of growth, differentiation, or metabolism. Likewise, pathological processes often have characteristic enzymatic activities that can be employed in their diagnosis, such as biomarkers, for example, cancer markers and cardiac enzymes.

Provided is a method for competitive inhibition of an enzyme for detecting a pathogen or a disease or a medical condition, or a biomarker thereof, in a subject. The method comprises providing a biological sample from the subject that may or may not contain an endogenous substrate. A test reaction is provided by contacting the biological sample with an enzyme indicative of the biomarker of a pathogen, disease, or medical condition and a substrate comprising a signaling moiety. The enzyme modifies the endogenous substrate and the substrate comprising the signaling moiety. Modification of the substrate comprising the signaling moiety by the enzyme produces a signal from the signaling moiety. Data from a control reaction comprising the enzyme and the substrate comprising the signaling moiety is further provided. The signal produced by the signaling moiety in the test reaction is detected. The presence of the biomarker of the pathogen, disease, or medical condition is indicated by a difference caused by the presence of the endogenous substrate in the biological sample between the signal produced in the test reaction and the data from the control reaction. The modification may comprise cleavage of the substrate or addition of a moiety to the substrate, such as a phosphate group. The method may also be used to detect a dysfunctional biological cascade in the subject by detecting an endogenous substrate in a biological sample that is modified by an enzyme participating in the biological cascade.

Also provided is a method of detecting a dysfunctional biological cascade in a subject using an array of competitive inhibition assays. The method comprises providing a biological sample from the subject that may or may not contain an endogenous substrate. An array of test reactions is further provided by contacting in each test reaction the biological sample with an enzyme that participates in the biological cascade and a substrate comprising a signaling moiety. The enzyme modifies the endogenous substrate and the substrate comprising the signaling moiety, and modification of the substrate comprising the signaling moiety by the enzyme produces a signal from the signaling moiety. Further provided is a reference profile from data from an array of control reactions comprising the enzyme and the substrate comprising the signaling moiety. The signal produced by the signaling moiety in the test reaction is detected and a sample profile of the signals produced from the array of test reactions is created. The sample profile is compared with the reference profile and the presence of a dysfunction of the biological cascade is indicated by a difference between the sample profile and the reference profile.

In another aspect, a method of detecting the presence or absence of a dysfunction of a biological cascade is provided using an array of test reactions that detects in a biological sample an enzyme that participates in the biological cascade. The method comprises providing the biological sample from a subject that may or may not contain the enzyme and providing an array of test reactions by contacting in each test reaction the biological sample with a substrate comprising a signaling moiety. The enzyme, if present, modifies the substrate comprising the signaling moiety and modification of the substrate by the enzyme produces a signal from the signaling moiety. The signal produced by the signaling moiety is then detected in the test reactions. The signal produced indicates the presence of a dysfunction in the biological cascade in the subject.

The biological cascade may be, for example, chosen from a coagulation cascade, fibrinolysis cascade, kinin cascade, signaling cascade, mitogen-activated protein kinase (MAPK) cascade, and inflammation cascade. Also, the pathogen, disease, or medical condition may be chosen from coagulation disorders, cancer, inflammation, neurodegenerative disorders, hypertension, vasodilation, diabetes, and allergy.

Also provided are methods for determining the effectiveness of a therapeutic treatment in a subject. The method comprises contacting two or more biological samples obtained from a subject at different time points of a therapeutic treatment with a substrate comprising a signaling moiety. An enzyme in the biological sample, if present, modifies the substrate and modification of the substrate by the enzyme produces a signal from the signaling moiety. The signal produced from the signaling moiety is detected and a difference in the signals produced from the two or more biological samples indicates the effectiveness of therapeutic treatment. The biological samples may be obtained before, during, and/or after treatment.

Also provided are methods for detecting enzymatic activity in a sample of peripheral white blood cells (WBC). The method comprises contacting a WBC sample that may or may not contain an enzyme obtained from a subject with a substrate comprising a signaling moiety. The enzyme modifies the substrate and modification of the substrate produces a signal from the signaling moiety. The signal produced is detected from the signaling moiety and the signal produced is indicative of enzymatic activity in the sample. The methods may be used to detect, for example, cytomegalovirus (CMV), human immunodeficiency virus (HIV), or human T-cell lymphotrophic virus (HTLV).

Also provided is a method for detecting the presence or absence of a fungal infection in a subject. The method comprises contacting a biological sample obtained from the subject that may or may not contain an enzyme produced from the fungus with a substrate comprising a signaling moiety. The enzyme modifies the substrate and modification of the substrate by the enzyme produces a signal from the signaling moiety. The signal produced from the signaling moiety is detected and the signal produced indicates the presence of a fungal infection in the subject. The fungus may be, for example, chosen from from Candida, Cryptococcus neoformans, Aspergillus fumigates, Blastocladiomycota, chytridiomycota, Dikarya, Glomeromycota, Microsporidia, and Neocallimastigomycota.

Also provided is a method for detecting the presence or absence of a meningitis infection in a subject. The method comprises contacting a biological sample obtained from the subject that may or may not contain an enzyme produced by a meningitis pathogen with a substrate comprising a signaling moiety. The enzyme modifies the substrate and modification of the substrate by the enzyme produces a signal from the signaling moiety. The biological sample is also contacted with one or more inhibitors of non-specific protease activity or meningitis protease activity. The signal produced from the signaling moiety is detected and the signal produced indicates the presence of a meningitis infection in the subject. The one or more inhibitors of meningitis protease activity, for example, inhibit bacterial meningitis but do not inhibit viral meningitis. As another example, the one or more inhibitors of meningitis protease activity inhibit pneumococcus protease activity but do not inhibit viral meningitis. In a further example, the one or more inhibitors of meningitis protease activity inhibit meningococcus protease activity but do not inhibit viral meningitis. In yet another example, the one or more inhibitors of meningitis protease activity inhibit both pneumococcus and meningococcus protease activities but do not inhibit viral meningitis. Thus, if a signal is produced in the presence of one or more inhibitors of meningitis protease activity that inhibit bacterial meningitis but do not inhibit viral meningitis, then the meningitis is a viral meningitis. On the other hand, if a signal is produced in the presence of one or more inhibitors of meningitis protease activity that inhibit pneumococcus protease activity and if a signal is not produced in the presence of one or more inhibitors of meningitis protease activity that inhibit meningococcus protease activity, then the meningitis is meningococcus. Similarly, if a signal is produced in the presence of one or more inhibitors of meningitis protease activity that inhibit meningococcus protease activity and if a signal is not produced in the presence of one or more inhibitors of meningitis protease activity that inhibit pneumococcus protease activity, then the meningitis is pneumococcus.

Also provided are methods for detecting the presence or absence of a disease or medical condition, or a biomarker thereof, in a subject. The disease or medical condition may be, for example, a dysfunctional endocrine system or prostate cancer. The method comprise contacting a biological sample obtained from the subject that may or may not contain an enzyme indicative of the disease or medical condition with a substrate comprising a signaling moiety. The enzyme modifies the substrate and modification of the substrate by the enzyme produces a signal from the signaling moiety. The signal produced from the signaling moiety is detected and the signal produced indicates the presence of a the disease or medical condition. The disease or medical condition may be, for example, a dysfunctional endocrine system and the enzyme may be, for example, aromatase. The disease or medical condition may also be, for example, prostate cancer, and the enzyme may be, for example, prostate specific cancer (PSA).

The methods provided may additionally comprise, for example, a separation step. In those embodiments, the substrate comprises a separation moiety and a signaling moiety allowing (1) separation between substrates that are processed in the reaction and substrates that are not processed and (2) detection of the processed substrates. Separation may be achieved by either specific binding of two moieties, such as between an antibody and antigen and between nucleic acids, or through binding to an immobilized surface, such as membranes, chips, and beads.

In a further aspect, the methods provided may include, for example, an amplification step. The method comprises contacting a biological sample with a first substrate fused to a first enzyme (called a zymogen) that becomes activated upon cleavage of the first substrate by the enzyme indicative of the pathogen, disease, or medical condition, or a biomarker thereof. The activated first enzyme modifies a second substrate comprising a signaling moiety and produces a signal from the signaling moiety. Cleavage of the zymogen produces a second active enzyme, which may activate another zymogen to produce a third active enzyme, and so forth. Each of the activated enzymes modifies a specific substrate. A signal is generated as result of each modification and therefore amplified. If a biological sample contains a substrate that competes with the cleavage sequence used to create the first zymogen, the signal generated will be reduced.

In some embodiments, the signaling moiety may be an enzyme, a fluorophore, a chromophore, a protein, a peptide, a chemiluminescent substance, a quencher, a Fluorescence Resonance Energy Transfer (FRET) pair, a pre-enzyme, and a radiosotope.

In some methods provided, one or more inhibitors of non-specific enzymatic activity may be added to the biological sample. In another aspect, one or more activators of an enzyme may be added to the biological sample.

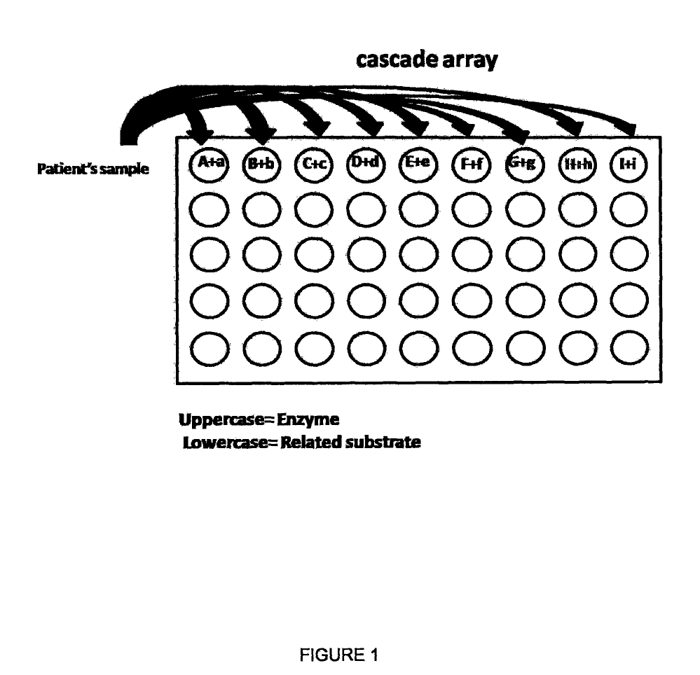

FIG. 1 is an example of an assay for a biological cascade.

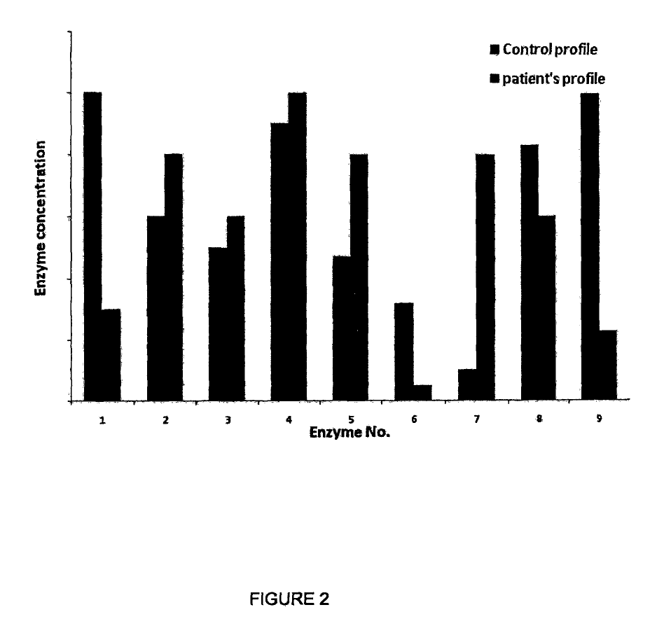

FIG. 2 is an example of a reference (control) profile and a sample (patient's) profile based on signals detected from an assay for a biological cascade.

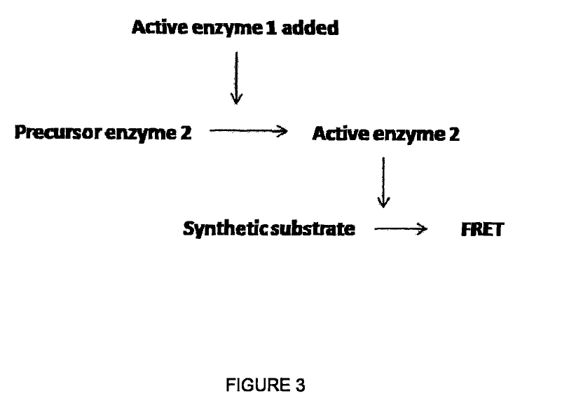

FIG. 3 is an example of an assay for a biological cascade utilizing inactive enzyme precurors (zymogens).

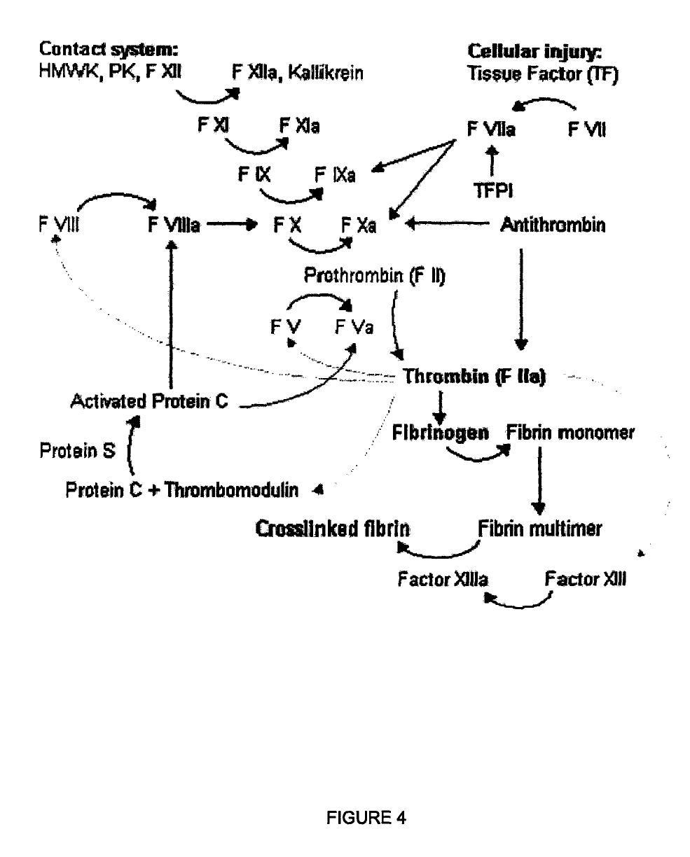

FIG. 4 is an illustration of a coagulation cascade.

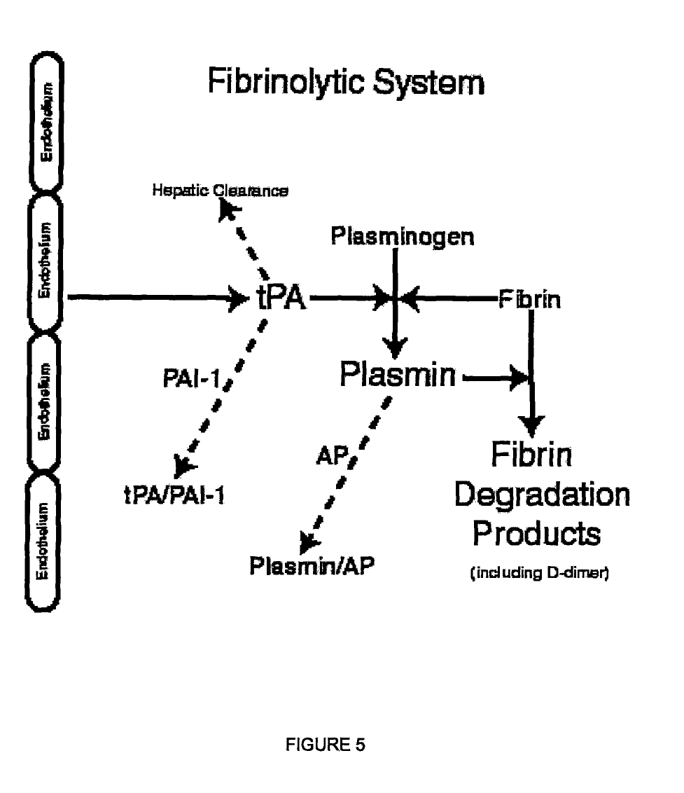

FIG. 5 is an illustration of a fibrinolysis cascade.

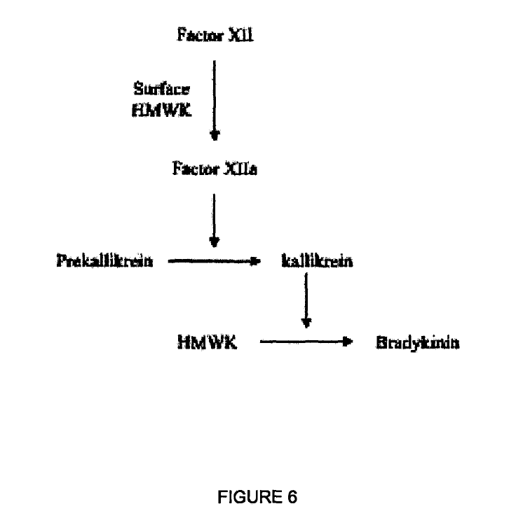

FIG. 6 is an illustration of a kinin cascade.

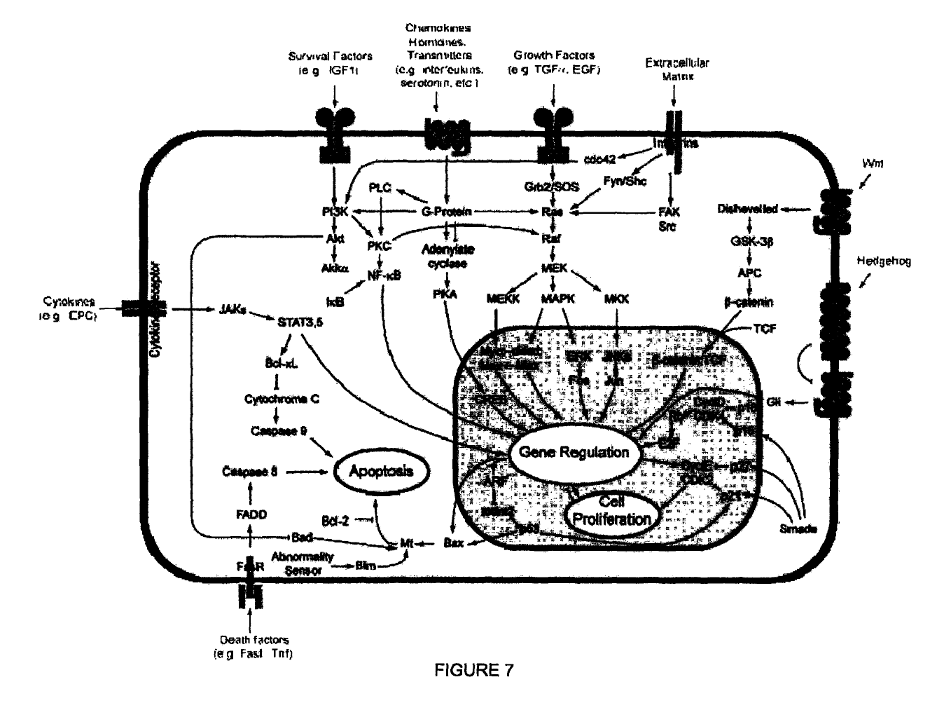

FIG. 7 is an illustration of a signaling cascade.

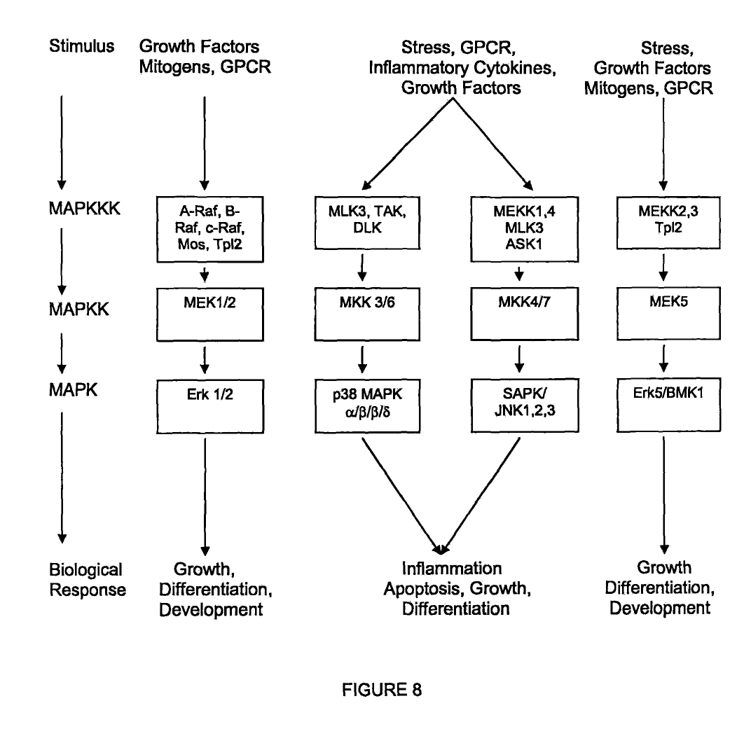

FIG. 8 is an illustration of a mitogen-activated protein kinase (MAPK) cascade.

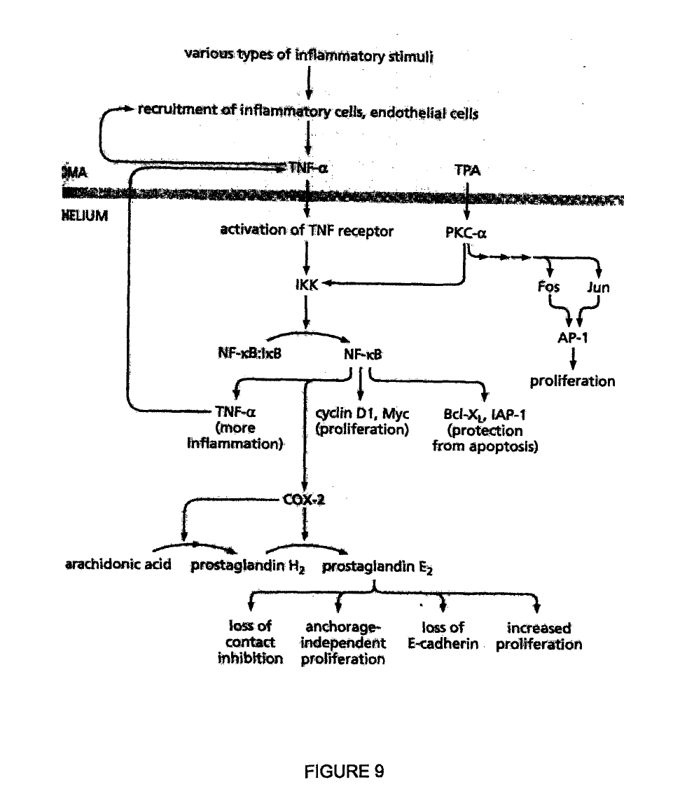

FIG. 9 is an illustration of an inflammation cascade.

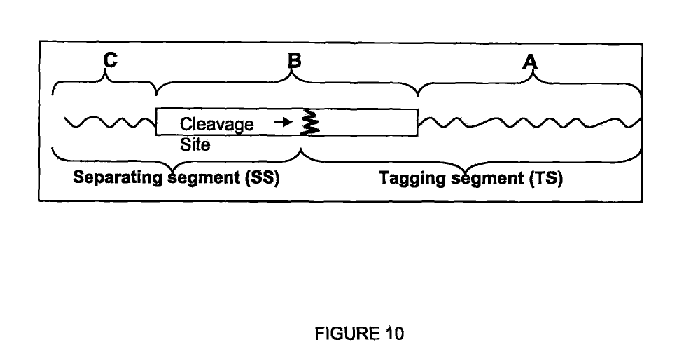

FIG. 10 is an illustration of the structure of a substrate.

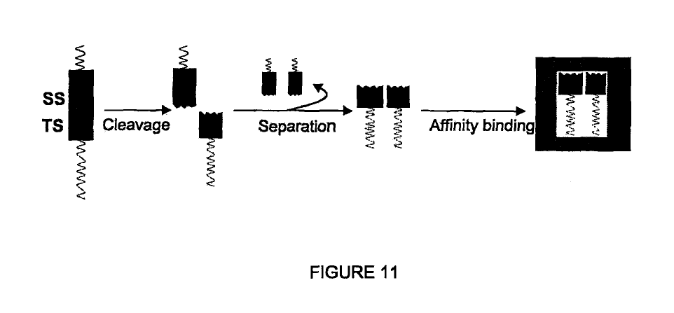

FIG. 11 is an illustration of an embodiment of the provided method comprising a separation step.

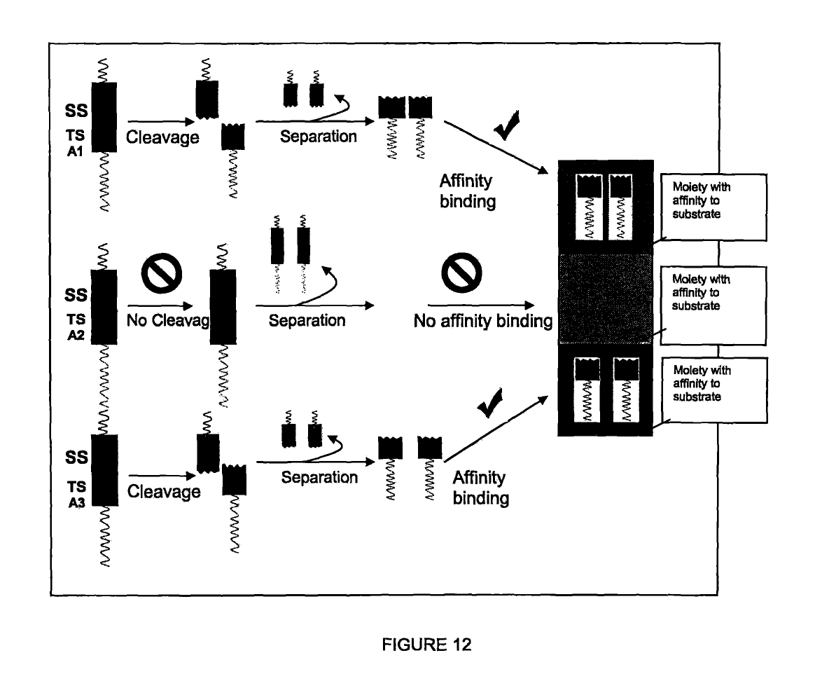

FIG. 12 is an illustration of an embodiment of the provided method for detecting cleavage of multiple substrates.

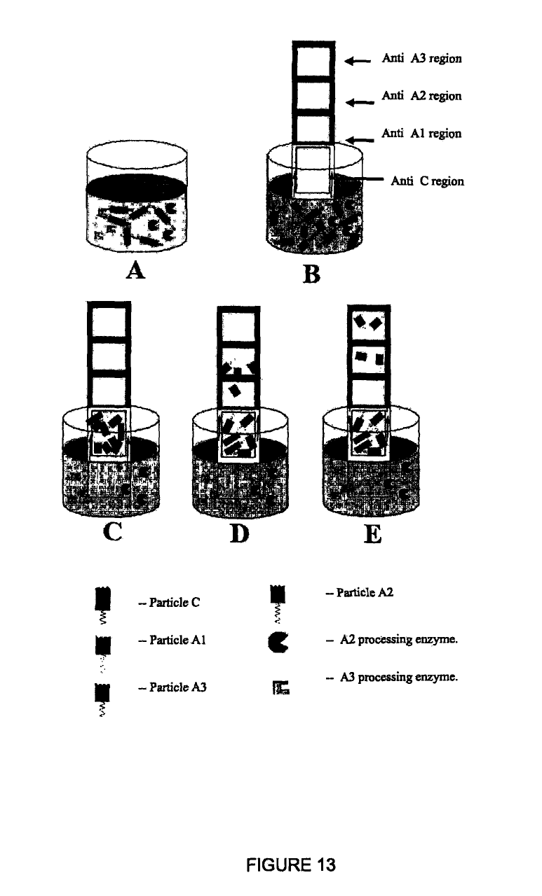

FIG. 13 is an illustration of a dynamic separation system.

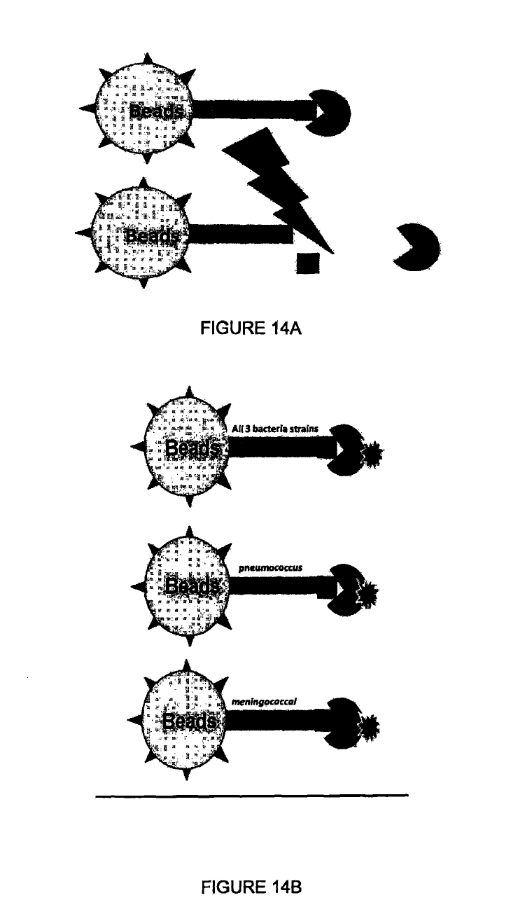

FIGS. 14A and 14B illustrate a method for detecting neuraminidase based on oligosaccharide beads and fluorescence labeled ligands (lectins). FIG. 14A shows the cleavage of the sialic acid and lectin by neuraminidase. FIG. 14B shows the various combinations of oligosaccharide/sialic acid/lectin combination for distinguishing between human, avian, and swine neuraminidases. SNA: Sambucus Nigra Lectin; MAL: Maackia amurensis lectin; WGA: Weat Germ Aglutinin.

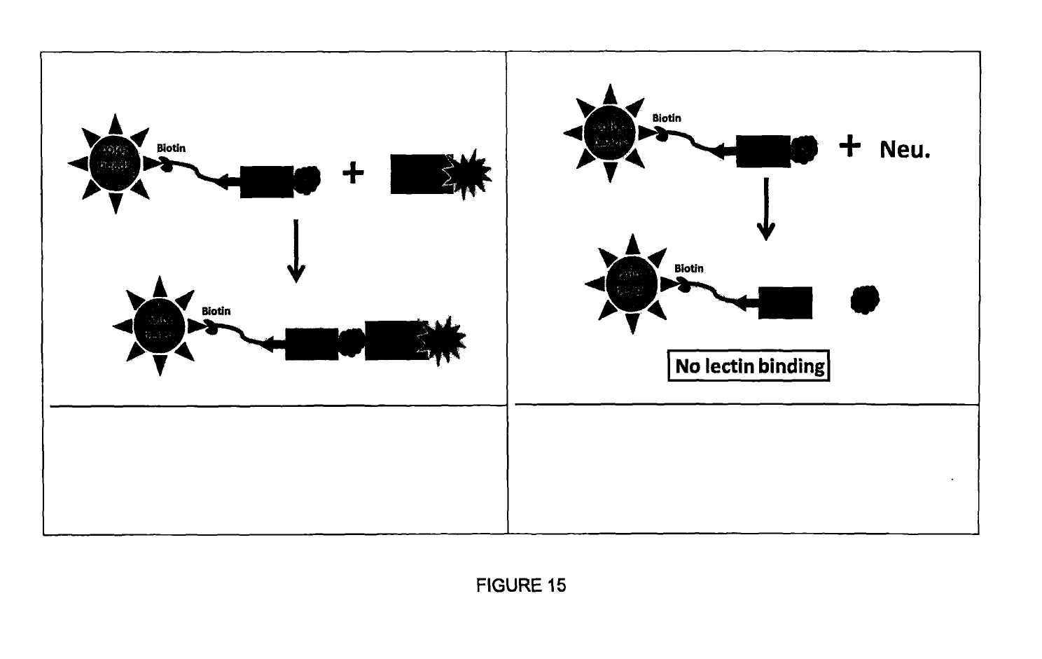

FIG. 15 is an illustration of a multiplex assay for neuraminidase detection based on oligossacharide beads and fluorescence labeled ligands (lectins).

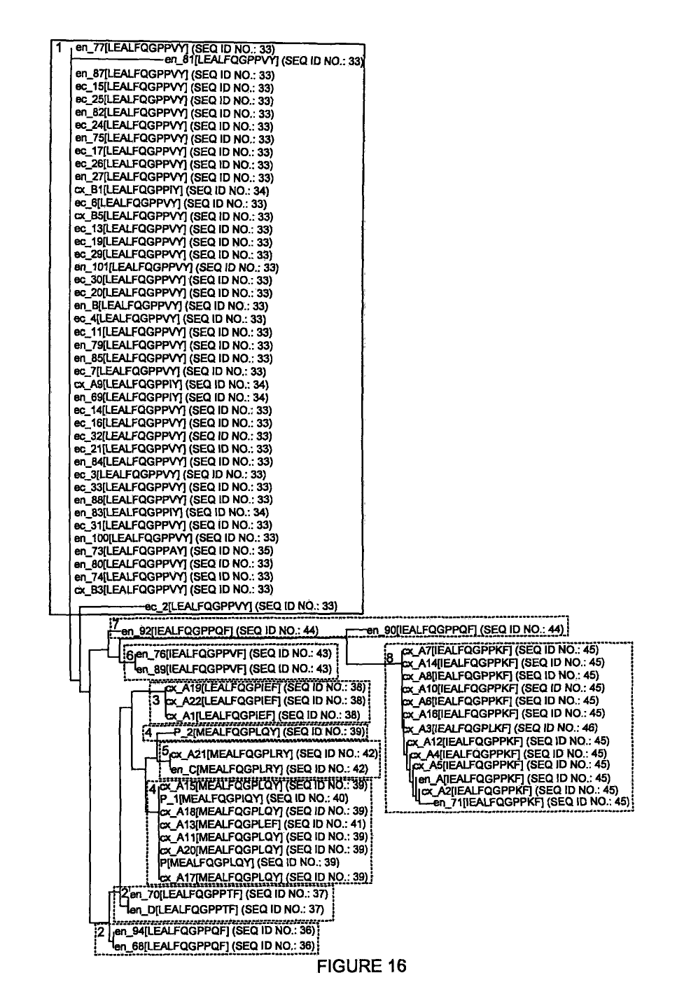

FIG. 16 is an evolutionary tree created according to 3D structure modeling of enterovirus 3C protease with it substrate Camb2.

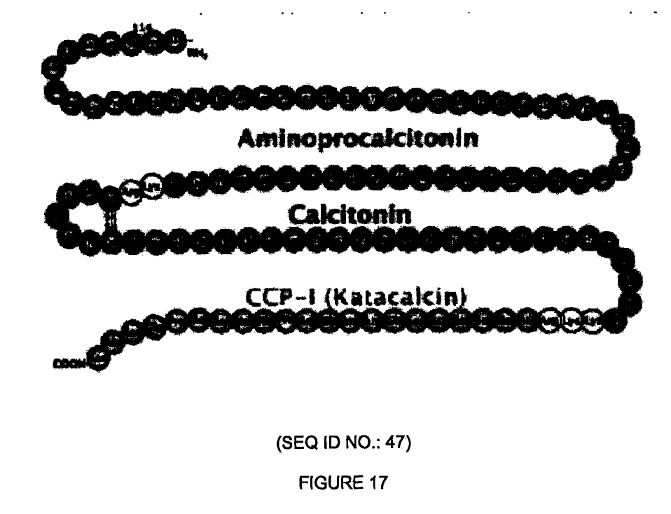

FIG. 17 is an illustration of the structure of procalcitonin (PCT).

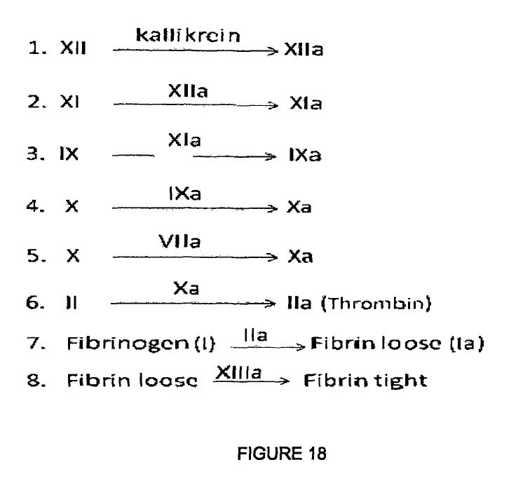

FIG. 18 is an example of an assay for the coagulation cascade.

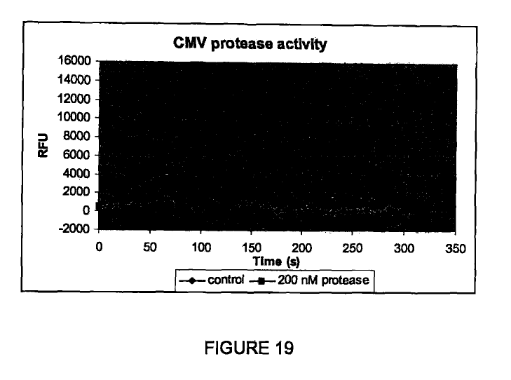

FIG. 19 shows the activity of 200 nM of recombinant CMV protease with 4 μM of the substrate Bach1.

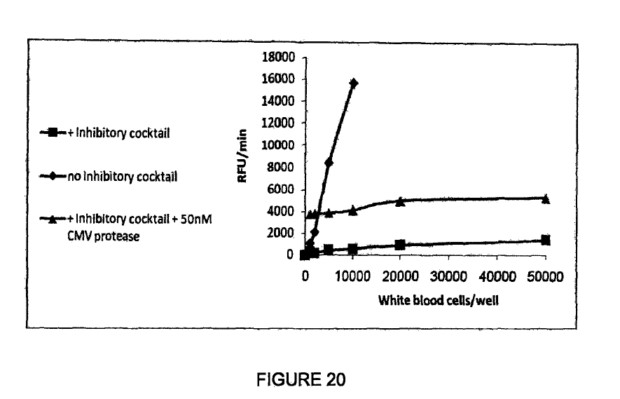

FIG. 20 shows WBC samples with and without inhibitors of non-specific protease activity and the effect of the inhibitors on CMV protease activity.

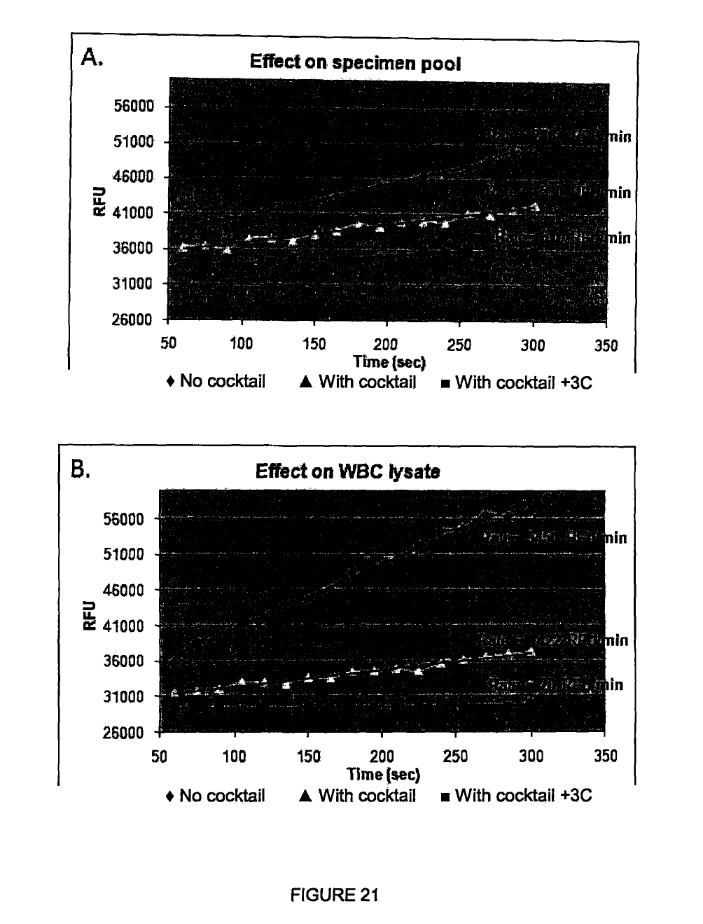

FIG. 21 shows the effect of an inhibitory cocktail on recombinant human rhinovirus 3C protease (3C) in specimen pools (FIG. 21A) and in WBC lysates (FIG. 21B).

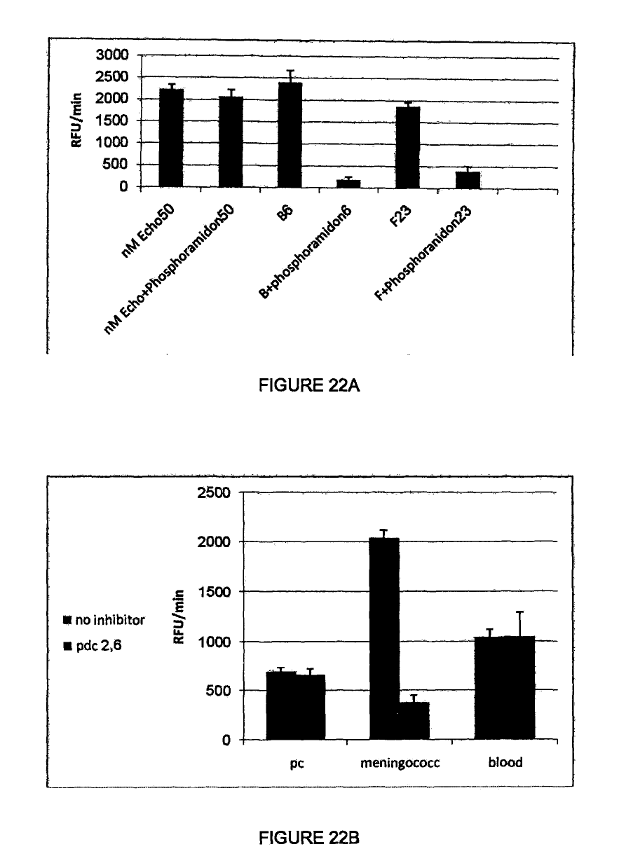

FIG. 22 shows the effect of inhibitors on viral and bacterial meningitis. FIG. 22A shows the effect of phosphoramidon on pneumococcus 6B and 23F but not on echovirus 3C protease. FIG. 22B shows the effect of 2,6-pyridinedicarboxylic acid on meningococcus but not on echovirus 3C protease.

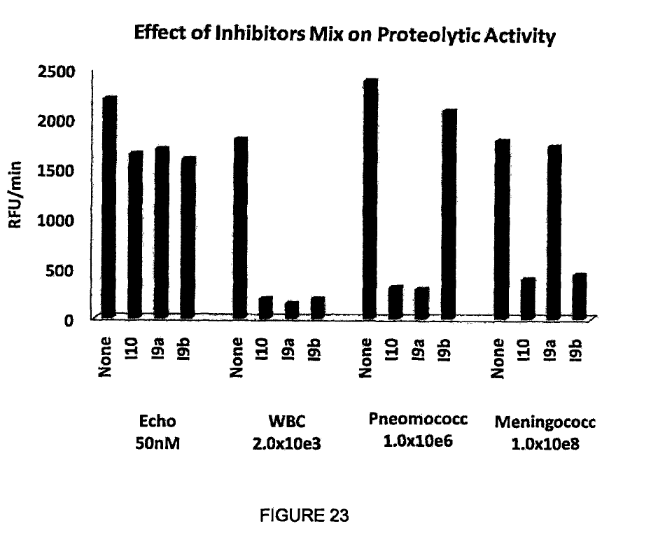

FIG. 23 shows the effect of inhibitor cocktails I10, I9a, and I9b on enterovirus 3C protease, pneumococc protease, and meningococc protease activities.

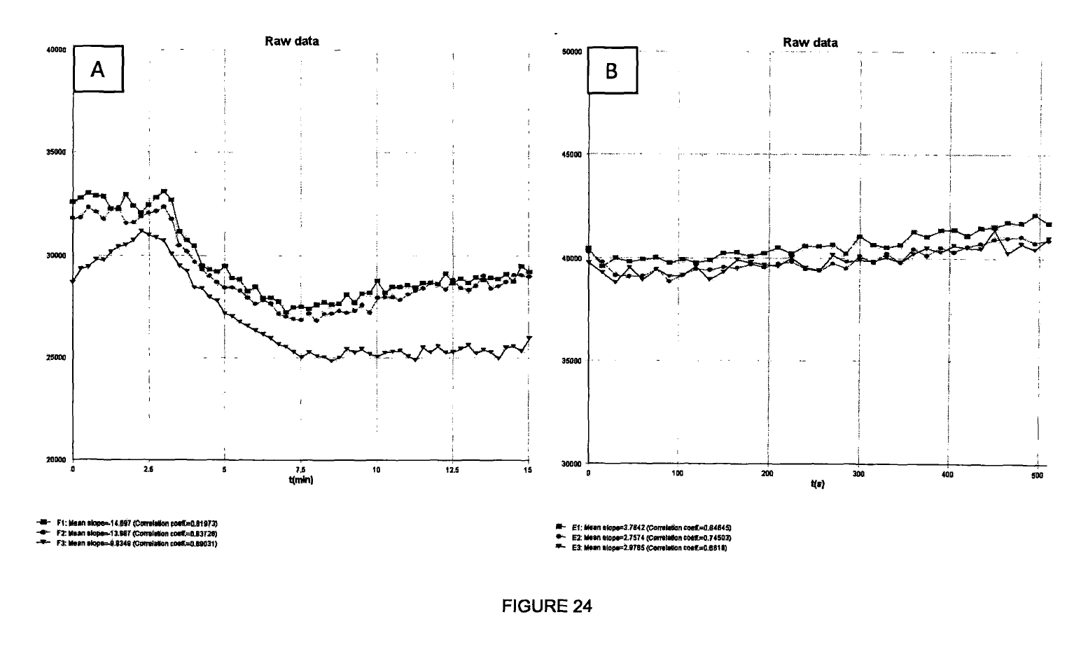

FIGS. 24A and 24B show the effect of nonbinding (NB) plates on the blank curve shape. FIG. 24A shows the wave shaped blank curve typical of regular plates and FIG. 24B shows the more linear shape with a slight positive slope with NB plates.

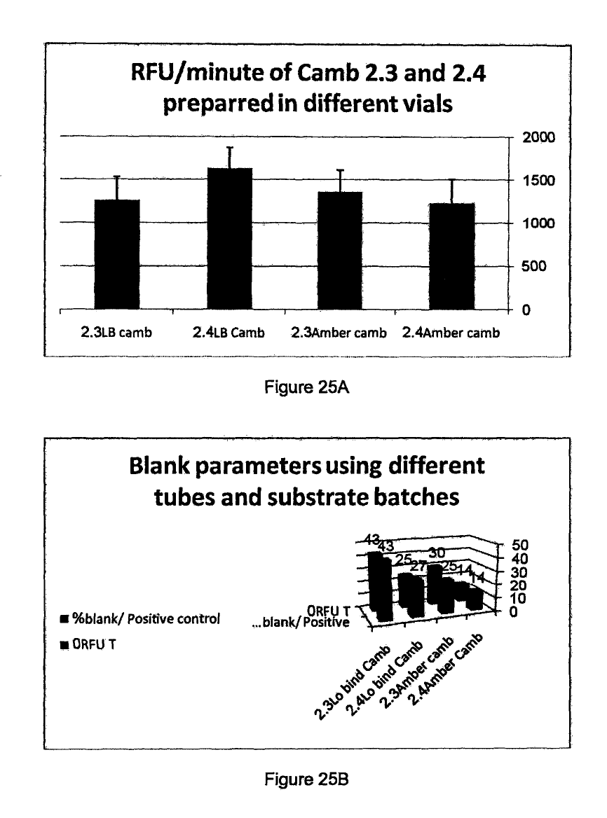

FIGS. 25A and 25B show the effect of different types of tubes for preparation of substrates on the enterovirus assay. FIG. 25A shows enterovirus activity using substrates Camb2.3 and Camb2.4 prepared in amber and low binding (1b) tubes. FIG. 25B shows the blank parameters of the enterovirus assay using different tubes and different substrate batches.

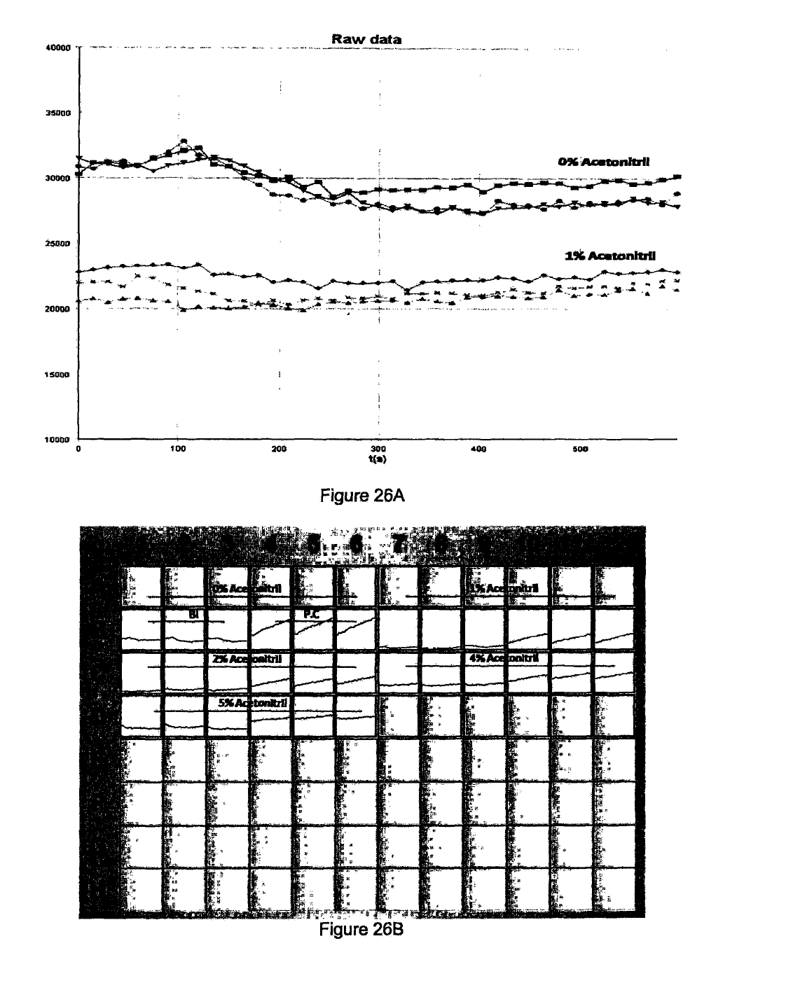

FIGS. 26A and 26B show the effect of acetonitrile on the enterovirus assay. FIG. 26A shows a blank comparison in the absence and in presence of 1% Acetonitril. FIG. 26B shows the effect of 0-5% Acetonitrile on enterovirus assay.

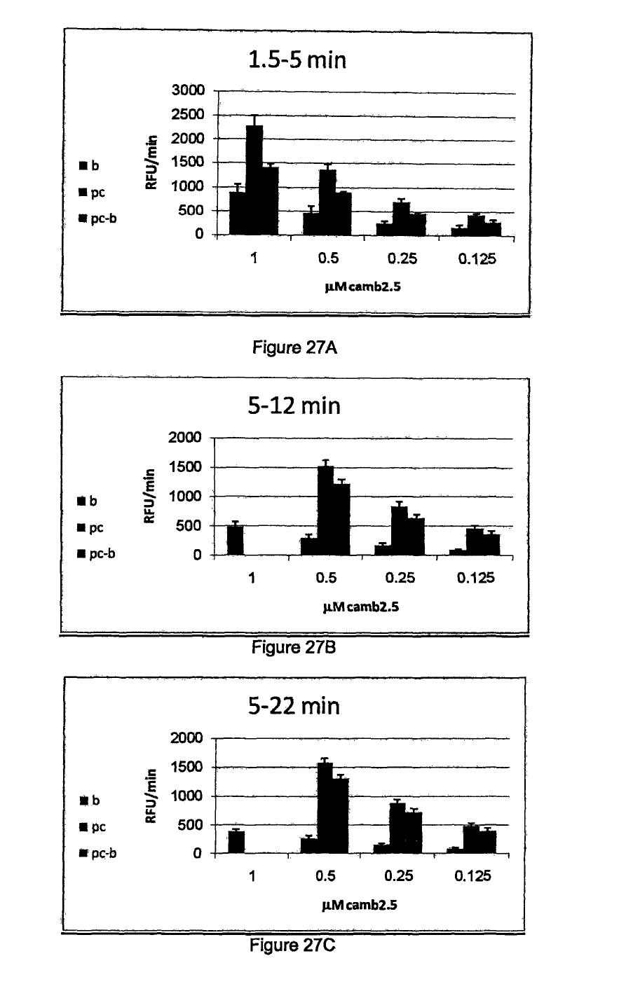

FIGS. 27A-27C show reaction rates in the enterovirus assay at different substrate concentrations (Camb2) and time intervals. FIG. 27A: 1.5-5 min; FIG. 27B: 5-12 min; FIG. 27C: 5-22 min. The data represent means of three experiments (each experiment was performed in triplicates)±S.E.

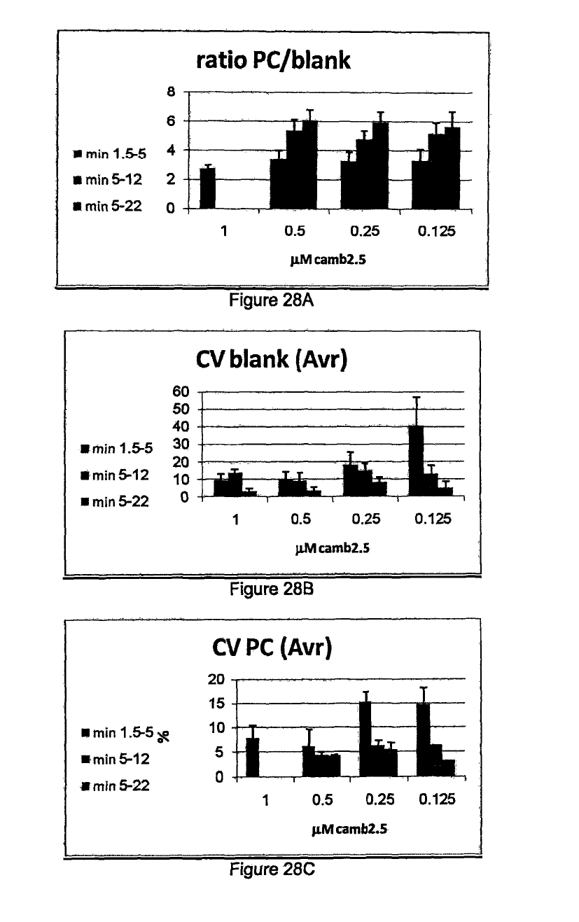

FIG. 28A shows the ratio between positive control and blank at different substrate concentrations (Camb2) and time intervals in the enterovirus assay. The data represent means of three experiments (each experiment was performed in triplicates)±S.E. FIGS. 28B and 28C show CV values of blank and positive control, respectively, at different substrate concentrations (Camb2) and time intervals in the enterovirus assay. The data represent means of three experiments (each experiment was performed in triplicates)±S.E.

As used herein, “biological sample” refers to any sample obtained from a subject, including, but not limited to, amniotic fluid mucus, saliva, throat wash, blood, white blood cells (WBC), serum, plasma, urine, cerebrospinal fluid (CSF), sputum, tissue biopsy, broncheoalveolar fluid, vaginal fluid, and tear fluid. In one aspect, red blood cells in a biological sample are removed before analysis by, for example, centrifugation. In another aspect, the red blood cells are removed by, for example, centrifugation, before freezing the sample.

As used herein, “biomarker” refers to a substance used as an indicator of normal biologic processes, pathogenic processes, or pharmacologic responses to a therapeutic intervention. A biomarker may include, for example, an antibody, peptide, protein, nucleic acid, an exogenous substance, or a chemical substance.

As used here, an “endogenous”substrate refers to a substrate that originates from an organism, tissue, or cell, and one that is not exogenously added to the biological sample being tested.

As used herein, “medical condition” refers to normal biological situations, such as pregnancy, that might benefit from medical assistance or have implications for medical treatments.

As used herein, “data from a control reaction” refers to a signal or analysis of a signal produced from a control reaction. A control reaction refers to a reaction that serves as a negative or positive control for a test reaction comprising a biological sample. Thus, for example, the control reaction may comprise an enzyme and a substrate comprising a signaling moiety that is modified by the enzyme but does not contain a biological sample. Alternatively, the control reaction may comprise an enzyme and a substrate comprising a signaling moiety, as well as a biological sample known to lack the biomarker being tested. Other control reactions are readily determinable by those skilled in the art. The data from a control reaction may be obtained simultaneously with a test reaction or may be obtained before or after performing a test reaction.

As used herein, “disease” refers to an abnormal medical condition of a subject that impairs bodily functions. Diseases include, but are not limited to, infections caused by, for example, fungi, yeast, or bacteria, cancer, auto-immune disorders, neurodegenerative disorders, allergies, cardiovascular disorders, and coagulation disorders.

As used herein, “enzyme” refers to any biomolecule that catalyzes chemical reactions. Enzymes include, but are not limited to, proteases, lipases, phospholipases, phosphatases, esterases, neuroaminidases, isomerases, hydrolases, polymerases, and helicases. Specific examples of enzymes include the viral proteases, neuraminidase, prostate specific antigen (PSA), and Sap2.

As used herein, “inhibitor” refers to any agent that abolishes or reduces the activity of an enzyme.

As used herein, “modify” refers to any chemical change in a substrate. Modification includes, but is not limited to, cleavage of the substrate and addition of a moiety such as the addition of a phosphate group.

As used herein, “pathogen” refers to an infectious agent that causes disease or an illness in a host. Pathogens include, but are not limited to, bacteria, viruses, yeast, and fungi.

As used herein, “procalcitonin” refers to full length 116 amino acid procalcitonin (SEQ ID NO: 47), or any of its naturally occurring truncated products, such as procalcitonin comprising 3-116 amino acids of SEQ ID NO:47, aminoprocalcitonin, immature calcitonin and calcitonin carboxypeptide-I (CCP-I or katacalcin).

As used herein, “separation moiety” refers to a moiety that allows separation of a component of the assay from another assay component. In an embodiment, a separation moiety is chosen from an immunological binding agent, a magnetic binding moiety, a peptide binding moiety, an affinity binding moiety, and a nucleic acid moiety.

As used herein, “signaling moiety” refers to any moiety that directly or indirectly produces a detectable signal. For example, the signaling moiety can be a detectable label that produces a fluorescencent, a chemiluminescent or a calorimetric signal. The signaling moiety may be chosen from an enzyme, a fluorophore, a chromophore, a protein, a peptide, a chemiluminescent substance, a quencher, a Fluorescence Resonance Energy Transfer (FRET) pair, a pre-enzyme, and a radiosotope. The signaling moiety may comprise an affinity pair.

As used herein, “affinity pair” refers to any two moieties that have affinity towards each other. Examples of affinity pairs include, but are not limited to, Biotin-Avidin; an Antibody-Substrate/antigen; Receptor-Substrate; Sialo-oligosacharid/ganglizides—lectins; Sense-Anti-sense DNA/RNA strands, based on nucleic acid hybridization; Nucleic acid Aptamers/target substrate; and pH dependent color molecule.

As used herein, “subject” refers to any person or non-human animal. The subject may be healthy or in need of treatment for a disease, disorder, or infection, or may refer to any subject for whom treatment may be beneficial. Non-human animals include all domesticated and feral vertebrates.

As used herein, “substrate” refers to a molecule that is capable of being modified by an enzyme. The substrate may be present in a biological sample. Or the substrate may be added to the test assay. In one embodiment, the substrate may comprise the general formula A-B, wherein B comprises a substance capable of being modified by an enzyme and A comprises a signaling moiety. In another embodiment, the substrate may comprise the general formula A-B-C, wherein B comprises a substance capable of being modified by an enzyme, and A and C each comprises a signaling moiety. The signaling moiety may be selected from an enzyme, a fluorophore, a chromophore, a protein, a peptide, a chemiluminescent substance, a quencher, a Fluorescence Resonance Energy Transfer (FRET) pair, a pre-enzyme, and a radiosotope. In yet another embodiment, A comprises a signaling moiety and C comprises a separation moiety. In a further embodiment, A comprises part of an affinity pair and C comprises a separation moiety. Substrates are well-known in the art and may be prepared according to the methods described in WO2005/01791, WO2007/029262, and WO 2007/049276, or any other methods known in the art. In addition, substrates may be designed by using 3D modeling of the enzyme, such as by modeling an enzyme bound to its substrate or an inhibitor.

In one embodiment, the substrate is a FRET based substrate. The fluorophore and quencher (the FRET pair) are attached at each side of the cleavage sequence of the substrate. Upon cleavage, the FRET becomes disassociated from the fluorophore such that fluorescence is emitted.

The substrate may be specific for one enzyme. The substrate may recognize multiple enzymes. For example, a single substrate may recognize multiple enzymes within a viral serotype but not of other serotypes, thereby distinguishing between serotypes.

As used herein, a sample of “white blood cells (WBC)” is any blood derived sample comprising WBC, such as at least 70% v/v WBC, further such as at least 80% v/v WBC, even further at least 90% v/v WBC, or even further such as at least 95%v/v WBC. Accordingly, the sample of WBC may include other components, such as bacteria and bacterial components.

The methods provided may be semi-quantitative by using control samples that contain a limited range of known enzyme and/or substrate concentrations. The methods may be quantitative by using control samples that contain a full range of known enzyme and/or substrate concentrations. Or the methods may be qualitative and may be observed by detecting a difference between the test sample and control sample.

### Enzymatic Assay Based on Competitive Inhibition for Detecting the Presence or Absence of a Substrate in a Biological Sample

In some embodiments, the method provided utilizes competitive inhibition of an enzyme for detecting the presence or absence of a pathogen or a disease or a medical condition, or a biomarker thereof, in a subject. The method comprises providing a biological sample from the subject that may or may not contain an endogenous substrate. A test reaction is further provided by contacting the biological sample with an enzyme indicative of the biomarker of a pathogen, disease, or medical condition and a substrate comprising a signaling moiety. The enzyme modifies the endogenous substrate and the substrate comprising the signaling moiety. Modification of the substrate comprising the signaling moiety by the enzyme produces a signal from the signaling moiety. Data from a control reaction comprising the enzyme and the substrate comprising the signaling moiety is also provided. The signal produced by the signaling moiety in the test reaction is detected. The presence of the biomarker of the pathogen, disease, or medical condition is indicated by a difference caused by the presence of the endogenous substrate in the biological sample between the signal produced in the test reaction and the data from the control reaction.

### Enzymatic Assay for the Detection of the Presence or Absence of a Dysfunction in a Biological Cascade

A biological cascade is a series of chemical reactions in which the products of one reaction are consumed in the next reaction. Examples of biological cascades include, but are not limited to, the coagulation cascade, the complement system, the signal transduction cascades, the fibrinolysis cascade, the apoptosis cascade, the MAPK cascade, the inflammation cascade, the kinin cascade, and the allergy cascade.

A dysfunction of one or more enzymes participating in a biological cascade may lead to a pathological medical condition. Some pathological medical conditions associated with a dysfunctional cascade result from an abnormal enzymatic activity of an enzyme participating in the cascade. An abnormal biological cascade may lead to the development of a pathological medical condition. Such pathological medical conditions may include, but are not limited to, coagulation disorders, cancer, inflammation, neurodegenerative disorders, hypertension medical conditions, vasodilation medical conditions, diabetes, and allergy.

In one embodiment, there is provided a method of determining in a biological sample the enzymatic activity of a plurality of enzymes participating in a biological cascade, both quantitatively and qualitatively. An activity profile of all the tested enzymes can be generated and correlated with a pathological medical condition. That profile allows not only the qualitative determination between a healthy or a pathological medical condition in the biological sample, but also allows the identification of one or more enzymes associated with the pathological medical condition and the nature of its dysfunction (e.g., lack of activity, increased activity, low activity etc.). The method is based on competitive inhibition as described above.

The activity of enzymes participating in a biological cascade can be determined using a competitive inhibition assay. A biological sample that may or may not contain an endogenous substrate is provided and contacted with an enzyme participating in the cascade and a substrate comprising a signaling moiety. The enzyme modifies the endogenous substrate and substrate comprising the signaling moiety and modification of the substrate comprising the signaling produces a signal from the signaling moiety. If the biological sample does not contain an endogenous substrate for the enzyme, there will be no competition with the substrate comprising the signaling moiety and a signal is produced from the signaling moiety as a result of modification of the substrate comprising the signaling moiety. If the biological sample contains an endogenous substrate for the enzyme, it will compete with the substrate comprising the signaling moiety and reduce the signal produced from the signaling moiety.

A dysfunction in a biological cascade may also be detected by using an array of competitive inhibition assays. The method comprises providing a biological sample from the subject that may or may not contain an endogenous substrate. An array of test reactions is further provided by contacting in each test reaction the biological sample with an enzyme that participates in the biological cascade and a substrate comprising a signaling moiety. The enzyme modifies the endogenous substrate and the substrate comprising the signaling moiety, and modification of the substrate comprising the signaling moiety by the enzyme produces a signal from the signaling moiety. Upon detecting the signal produced, a “sample profile” comprising signal levels from each assay may be created and compared with a reference profile. As used herein, “reference profile” refers to an activity profile of each enzyme participating in a cascade. The enzymatic activity of the various enzymes may be presented as a histogram, a pie, or by any other means for presenting enzymatic activity. A difference between the sample profile and the reference profile indicates a dysfunction in the cascade. The difference may also indicate a pathological medical condition in the subject.

In another embodiment, the presence or absence of a dysfunction of a biological cascade is detected using an array of test reactions that detects in a biological sample an enzyme that participates in the biological cascade. The method comprises providing the biological sample from a subject that may or may not contain the enzyme and providing an array of test reactions by contacting in each test reaction the biological sample with a substrate comprising a signaling moiety. The enzyme, if present, modifies the substrate comprising the signaling moiety and modification of the substrate by the enzyme produces a signal from the signaling moiety. The signal produced by the signaling moiety is then detected in the test reactions. The signal produced indicates the presence of a dysfunction in the biological cascade in the subject.

In yet another embodiment, there is provided a method of determining effectiveness of a therapeutic treatment of a pathological medical condition in a subject. In this instance, at least two arrays are utilized. The arrays test for activity of enzymes in a biological cascade with biological samples obtained at different time points before, during, or after treatment. Thus, for example, for one array, the biological sample tested may be obtained before beginning treatment, and for another array, the biological sample may be obtained at the completion of treatment. In another example, biological samples may be obtained during treatment but at different time points. In yet another example, a biological sample may be obtained before treatment, during treatment, and after treatment.

Sample profiles for each array may be compared to a reference profile. A decrease in the difference between the profiles with treatment indicates effectiveness of treatment.

In another embodiment, biological cascades may be tested as illustrated in FIG. 1. To each well of the array, a biological sample that may or may not contain an endogenous substrate is contaced with an enzyme that participates in the cascade and a substrate comprising a signaling moiety. Fluorescence in each well is measured and activity profiles are created as shown in FIG. 2.

In another embodiment, an inactive precursor of the enzyme participating in the biological cascade may be utilized. An example of the embodiment is shown in FIG. 3. To activate the enzyme, a second enzyme is added that activates the first inactive enzyme. The activated enzyme then can act on one or more substrates.

### Examples of Biological Cascades

Coagulation Cascade

Coagulation is a complex process by which blood forms clots. It plays a role in homeostasis (the cessation of blood loss from a damaged vessel). Coagulation is initiated almost instantly after an injury to the blood vessel in the endothelium. Platelets immediately form a haemostatic plug at the site of injury. Later, proteins in the blood plasma called coagulation factors respond in a complex cascade to form fibrin strands which strengthen the platelet plug.

The coagulation cascade has two pathways, the contact activation pathway (called the intrinsic pathway) and the tissue factor pathway (called the extrinsic pathway) that lead to fibrin formation. See FIG. 4 The pathways are a series of reactions in which a zymogen (an inactive enzyme precursor) of a serine protease and its glycoprotein co-factor are activated to become active components that then catalyze the next reaction in the cascade, ultimately resulting in cross-linked fibrin. Serine proteases act by cleaving other proteins at specific sites. The coagulation factors circulate as inactive zymogens. Table 1 lists a number of coagulation factors.

TABLE 1
Plasma Coagulation Factors

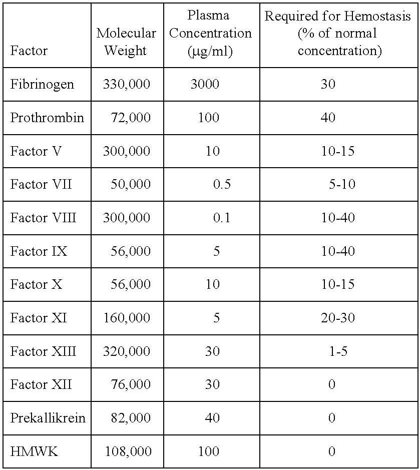

Structure of the Coagulation Protease Zymugens

The protease zymogens involved in coagulation are secreted into the bloodstream by hepatocytes and contain a signal peptide that is removed during transit into the endoplasmic reticulum. About 200 amino acids at the c-terminal end of each zymogen are homologous to trypsin and contain the Ser, Asp, His residues of the active site of the protease. Those domains appear to be involved in specific interactions between the proteases and their substrates, cofactors and/or inhibitors.

Non-Enzymatic Protein Cofactors

Non-enzymatic protein cofactors include factor V and VIII, tissue factor and high-molecular weight kininogen (HMWK). See Table 2 below. Factors V and VIII are large plasma proteins that contain repeated sequences homologous to the copper-binding protein ceruloplasmin. Thrombin cleaves factors V and VIII to yield activated factors (Va and VIIIa). Factors Va and VIIIa have no enzymatic activity. Instead, they serve as cofactors that increase the proteolytic efficiency of Xa and IXa, respectively.

Tissue factor is a non-enzymatic lipoprotein constitutively expressed on the surface of cells that are not normally in contact with plasma. It is expressed on the surface of “activated” monocytes and endothelial cells exposed to various cytokines such as tumor necrosis factor. Tissue factor greatly increases the proteolytic efficiency of VIIa.

TABLE 2
Non-enzymatic Protein Cofactors

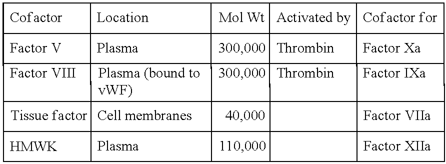

The Fibrinolysis Cascade

The fibrinolysis cascade (see FIG. 5) acts in opposition to the coagulation system and involves degrading the fibrin clot when it is no longer needed. It also serves to prevent extension of a clot beyond the site of injury. Fibrinolysis is initiated by tPA (tissue plasminogen activator) or uPA (urokinase-like plasminogen activator), which converts plasminogen to plasmin in the presence of fibrin by cleaving the Arg561-Va1562 peptide bond in plasminogen. Plasmin degrades the fibrin clot and intact fibrinogen to soluble fibrin/fibrinogen degradation products (FDP). Plasmin also inactivates factors Va and VIIIa (as does Protein C and Protein S). tPA is produced by endothelial cells; activation of plasminogen is major mechanism for lysis of fibrin clots. Recombinant tPA is used to treat myocardial infarction, stroke, and in some cases, acute thrombosis. uPA is produced by urine and plasma; it keeps renal tracts free of blood clots. It also plays a role in initiating nonfibrinolytic activities of plasmin. Excessive fibrinolysis is regulated by a plasmin inhibitor (antiplasmin, formerly called alpha2-antiplasmin) and plasminogen activator inhibitor 1 (PAI-1). PAI-1 is synthesized by hepatocytes and endothelial cells, is present in platelets and plasma, and can bind to fibrin and inhibit plasminogen activators tPA and uPA. PAI-1 is an acute phase reactant protein, and may increase 30-50 fold over baseline, possibly immediately inactivating systemically administered tPA. Homozygous deficiency of plasminogen is associated with ligneous conjunctivitis (a rare form of chronic pseudomembranous conjunctivitis), and replacement therapy with plasminogen is therapeutic. Neither heterozygous plasminogen deficiency (0.5 to 2.0% of subjects with thrombosis) nor tPA deficiency is associated with increased risk of thrombosis.

The Kinin Cascade

The kinin-kallikrein cascade (or the kinin cascade) plays a role in inflammation, blood pressure control, coagulation, and pain. See FIG. 6. Their mediators bradykinin and kallidin are vasodilators and act on many cell types. Kinins are small peptides, and tissue injury induces activation of these peptides, resulting in vasodilation and increased permeability. A function of kallikrein is to amplify the activation of coagulation and the fibrinolytic cascades. Kallikrein also cleaves high molecular weight kininogen (HMWK) to produce bradykinin, a potent inflammatory mediator that produces vasodilation during the recruitment of leukocytes.

Signaling Cascade

Apoptosis is a form of programmed cell death (PCD) in multicellular organisms. It is a type of PCD and involves a series of biochemical events leading to a characteristic cell morphology and death. Morphological changes include blebbing, changes to the cell membrane such as loss of membrane asymmetry and attachment, cell shrinkage, nuclear fragmentation, chromatin condensation, and chromosomal DNA fragmentation. Processes of disposal of cellular debris whose results do not damage the organism differentiate apoptosis from necrosis.

The caspases, which are cysteine proteases that are homologous to the C. elegans ced-3, play a role in the apoptotic signaling cascade that is activated in most cases of apoptotic cell death. The catalytic activity of caspases depends on a cysteine residue within a highly conserved pentapeptide QACRG. The caspases specifically cleave their substrates after Asp residues.

The signaling cascade involving caspases is depicted in FIG. 7. Both the extrinsic and the intrinsic pathways lead to apoptosis. In many pathological processes, a dysfunction in the apoptosis cascade can lead to uncontrolled proliferation and cancer.

The MAPK Cascade

Mitogen-activated protein (MAP) kinases are serine/threonine-specific protein kinases that respond to extracellular stimuli (mitogens) and regulate various cellular activities, such as gene expression, mitosis, development, differentiation, transmission of oncogenic signals and cell survival/apoptosis.

MAPK is involved in the action of most nonnuclear oncogenes. It is responsible for cell response to growth factors such as brain-derived neurotrophic factor (BDNF) or nerve growth factor. Extracellular stimuli lead to activation of a MAP kinase via a signaling cascade composed of MAP kinase, MAP kinase kinase (MKK, MEKK, or MAP2K), and MAP kinase kinase kinase (MKKK or MAP3K). See FIG. 8.

A MAP3K that is activated by extracellular stimuli phosphorylates a MAP2K on its serine and threonine residues, and then MAP2K activates a MAP kinase through phosphorylation on its serine and tyrosine residues.

All MAPK pathways operate through sequential phosphorylation events to phosphorylate transcription factors and regulate gene expression. They can also phosphorylate cytosolic targets to regulate intracellular events.

The Inflammation Cascade

The inflammation cascade is a complex biological response of vascular tissues to harmful stimuli, such as pathogens, damaged cells, or irritants. It is a protective attempt by the organism to remove the injurious stimuli as well as initiate the healing process for the tissue. An example of a model of an inflammation cascade in endothelial cells is shown in FIG. 9.

### Enzymatic Assay for Detecting the Presence or Absence of an Enzyme in a Biological Sample

A method for detecting enzymatic activity in a biological sample is well-known and has been described in WO2005/01791, WO2007/029262, and WO 2007/049276. Detection of enzymatic activity indicates the presence of a pathogen, disease, or medical condition, or a biomarker thereof in a subject from which the biological sample was obtained. The method generally comprises contacting a biological sample obtained from a subject that may or may not contain an enzyme with a substrate of the enzyme to be detected. The substrate comprises a signaling molecule such that when the enzyme is present in the biological sample, the enzyme modifies the substrate and the signaling moiety emits a signal, indicating the presence of a pathogen, disease, or a medical condition, or a biomarker thereof, in the subject. In one embodiment, multiple enzymes may be detected in a biological sample using an array.

Some non-limiting pathogens, diseases, and medical conditions to be detected by the provided methods include those caused by fungi, yeast, bacteria, cancer, auto-immune disorders, neurodegenerative disorders, and allergies. In addition, the method provided can also be utilized for the diagnostic of cancerous medical conditions, genetic diseases, heart medical conditions (e.g. cardiovascular disorders) and coagulation disorders.

### Determining the Effectiveness of a Treatment

In addition to detecting a pathogen, disease, or medical condition, or a biomarker thereof, in a subject, the methods provided may be useful for determining the effectiveness of a treatment for the pathogen, disease, or medical condition. Biological samples may be obtained at different time points before, during, or after treatment and subjected to the enzymatic assay of the present methods. In one embodiment, a biological sample is obtained prior to treatment, and another is obtained during treatment. In another embodiment, a biological sample is obtained before treatment, and another is obtained after completion of treatment. In yet another embodiment, two or more biological samples are obtained during treatment. A difference, such as a reduction, in the signals produced from the two or more biological samples is indicative of the effectiveness of therapeutic treatment.

### Separation Step

Any of the methods provided can include a separation step. In this embodiment, the method comprises: 1) separation between substrates that are processed in the reaction and substrates which are not processed; and 2) detection of processed substrates only. Separation may be achieved by either specific binding of two moieties, such as between an antibody and antigen and between nucleic acids, or through binding to an immobilized surface, such as membranes, chips, and beads. The detection step can be based on affinity or via a signaling moiety, or both.

The substrate used in the method that comprises a separation step is comprised of three parts: A,B and C (FIG. 10). The core molecule (segment B), which has a specific cleavage site, is associated at one end to a signaling moiety (A), which serves to detect cleaved substrates. At the other end, segment B is connected to a separation moiety (C) that separates between processed and unprocessed substrates. Upon cleavage of molecule B, the substrate produces two fragments: (1) Signaling moiety (TS) that contains part A and a part of B and (2) a Separation moiety (SS) that contains part C and a part of B.

FIG. 11 shows an embodiment of a method provided. The substrate reacts with its enzyme and upon cleavage, segment C is used to separate between the processed and unprocessed substrates. Segment A in this instance is part of an affinity pair. When processed, only the TS segment of the processed substrate (that contains the tagging molecule) binds to its affinity pair. The affinity binding process is therefore detected only for cleaved substrates. In this way it is possible to detect only molecules that were processed.

Another embodiment is shown in FIG. 12. In this embodiment, the cleavage of multiple substrates can be detected using the above described method if the substrates are similar in their separation moiety (C) but differ in their specific cleavage molecule (B). In this case, each substrate has a unique and different signaling moiety (A) that can be associated with the core molecule comprising the cleavage site (B). After cleavage and separation between processed and unprocessed substrates, only the TS of the different (and processed) substrates are bound by affinity (in accordance to the above described method). Any molecule that contains C (unprocessed substrates or SS of processed substrates) is separated out by the separation moiety. The different TSs of different substrates may be distinguished by immobilizing its binding partner to a predetermined location on a solid surface, such as a membrane, well, or chip, such that each location can bind only to one kind of TS. By knowing which TS should bind to the predetermined location, the substrates processed can be identified. Because each substrate is specific to the enzyme that initiated the substrate cleavage, the enzyme can be identified, allowing the deduction of which pathogen, disease, or medical condition, or biomarker thereof, is present in the subject.

Yet another embodiment utilizes the Reverse pH System (RPHS). In this embodiment, the C segment is a molecule common to all substrates. The A segments that are associated with the various substrates are dye molecular entities in which their different dyes are sensitive to different pHs. After cleavage, the reaction mixture is filtered through a column with affinity to segment C. Any molecule that contains segment C of FIG. 10 (unprocessed substrates or segment SS of processed substrates) will be retained at the column. Only the TS segment of the processed substrates (that does not contain segment C) will be transferred to a chamber that has a number of cells, each having different pHs. Once the TS segment (that contains A) comes in contact with the cells having different pHs, the cell changes color according to the properties of segment A. This indicates which substrates have been processed.

Other examples of separation systems include the following:
1. Immobilized Separation System (ISS)—In this embodiment, segment C in FIG. 10 is a spacer linked to an immobilized surface via beads, nitrocellulose membrane, biotin-avidin or other affinity pair. After cleavage, any unprocessed substrate or the SS of the processed substrate is removed by separating the immobilized surface (by extraction, centrifugation, filtration etc.) from the reaction mixture, leaving only the TS of the processed substrates. This method also allows monitoring the kinetics of each substrate.
2. Dynamic Separation System (DSS)—This embodiment is shown in FIG. 13. In this system, segment C in FIG. 10 is a special molecule common or unique to all substrates. After cleavage, the reaction mixture contacts a solid surface, such as a membrane or chip. The membrane is vertical and comprises a moiety with affinity to segment C at the bottom. Other parts of the membrane include different loci comprising a moiety with affinity to segment A of the different substrates. The reaction mixture is then pushed along the length of the membrane or chip by capillary or electro force. Any molecule that contains C (unprocessed substrates or SS of processed substrates) will be retained at the bottom of the membrane. Only the TS of the processed substrates (that do not contain C) will be able to move up the membrane and bind by affinity to their predetermined loci.
3. Affinity Filtration System (AFS)—In this embodiment, the reaction mixture is filtered through a column with affinity to C, thus any molecule that contains C (unprocessed substrates or SS of processed substrates) will remain in the column. The flow through will contain only the TS of the processed substrates.

### Detection System

Modification of substrates can be detected by a number of methods. Examples of detection methods include the following:
Antibody/Receptor-substrate—Immunochemistry can be used to detect and measure binding between the antibody or receptor to the substrate.
Ligand/Receptor-substrate—Immunochemistry can be used to detect and measure binding between the ligand or receptor to the substrate.
Marker—The signaling moiety (A) of FIG. 10 can be a molecule that produces color, fluorescence, FRET or any other measurable, visible or easily detectable molecule.
DNA/RNA Hybridization—Hybridization can be detected and measured, for example, by fluorescence or use of a color probe.
Enzymatic reaction—The signaling moiety (A) can be an enzyme that catalyzes color or fluorescence or any other measurable, visible or any other easily detectable reaction.

### Reaction Conditions

Reaction conditions can be optimized to increase specificity and/or sensitivity of the methods of the invention. Examples of reaction conditions that may be optimized include reaction temperature, reaction time, solvent, buffer, plates, and tubes. Thus, for example, the reaction may be performed at ambient temperature, including room temperature and body temperature. Examples of optimization for the enterovirus assay in CSF samples are provided in Examples 16-21 below. Moreover, the methods of the invention may be performed in a laboratory setting or in field conditions.

### Inhibitor

In some embodiments, non-specific modification of substrates may be high such that it impedes the detection of enzymatic activity. The source of this activity may be due to the presence of non-specific enzymes being able to modify the substrate. The human genome encodes for hundreds of enzymes, some of which have no apparent specificity. Many of these have been identified in the biological samples based on comprehensive bioinformatics and each tissue/organ source has been associated with a number of these non-specific enzymes. Exemplary tissue/organ sources include muscular, urinary, respiratory, digestion, neurological, reproduction, skin, circulatory, skeletal, and endocrine. Each non-specific enzyme can be analyzed for their target sequence and compared to the sequence in the substrate of interest. An inhibitor or a cocktail of inhibitors can be selected and added to the samples to inhibit the activity of non-specific enzymes, while having minimal effect on the activity of the enzyme of interest. An example of such inhibitors includes, but is not limited to, Pestatin A, AEBSF, Aprotinin, E-64, Heparin, Bestatin, GW311616A and eglin C for inhibiting non-specific activity against a CMV protease substrate in a sample of white blood cells. Another example of inhibitors includes, but is not limited to, E-64, Pepstatin A, Aprotinin, Acetyl-DEVD-CHO, EDTA +EGTA, AEBSF, Eglin C, and Bestatin for inhibiting non-specific activity against a human rhinovirus (HRV) substrate in a nasal wash sample.

In addition, background noise may be caused indirectly by the pathogen. For example, many pathogens induce inflammation, which can induce various enzymatic reactions within the body that may impede detection of an enzyme or substrate produced directly by the pathogen. Thus, in one embodiment, one or more inhibitors may be useful for inhibiting enzymatic activity associated with an infection. In a specific embodiment, the one or more inhibitors inhibits enzymatic activity as a result of meningitis induced inflammation.

In a specific embodiment, one or more inhibitors may be useful for distinguising between viral and bacterial infections, for example, between viral and bacterial meningitis. The one or more inhibitors can be selected to inhibit bacterial meningitis protease activity but not viral meningitis protease activity. Thus, when a biological sample obtained from a subject is contacted with a substrate capable of being modified by a meningitis protease in the presence of the one or more inhibitors selective against bacterial meningitis protease, the signal produced is indicative of a viral meningitis infection in the subject. The one or more inhibitors can also be used to distinguish between bacterial infections causing the same disease or medical condition. For example, the one or more inhibitors can be used to distinguish between pneumococcus and meningococcus infections, both of which cause bacterial meningitis infections. In this embodiment, one or more inhibitors can be selected to inhibit pneumococcus protease activity but not viral meningitis. Another one or more inhibitors can be selected to inhibit meningococcus protease activity but does not viral meningitis. In yet another embodiment, the one or more inhibitors can be selected to inhibit both pneumococcus and meningococcus protease activities but not viral meningitis. Thus, if a signal is produced in the presence of one or more inhibitors that inhibits pneumococcus protease activity and if a signal is not produced in the presence of one or more inhibitors that inhibits meningococcus protease activity, then the meningitis is meningococcus. Similarly, if a signal is produced in the presence of one or more inhibitors that inhibits meningococcus protease activity and if a signal is not produced in the presence of one or more inhibitors that inhibits pneumococcus protease activity, then the meningitis is pneumococcus.

### Activator

In some embodiments, an activator may be added to the reaction mixture. As used herein, “activator” refers to any agent that induces or increases the activity of an enzyme. In an embodiment, the activator is Na₂SO₄.

Amplification of Signal

Levels of an enzyme or substrate in a biological sample may, in some instances, be low or even below detection level. For example, procalcitonin levels during bacterial infection can be as low as 0.5 ng/ml (40 μM). In order to detect such low levels, amplification of a signal maybe useful. Amplification of a signal may be achieved by utlizing a zymogen activation cascade. A zymogen, or a proenzyme, is an inactive enzyme precursor. A zymogen requires a biochemical change (such as a hydrolysis reaction revealing the active site, or changing the configuration to reveal the active site) for it to become an active enzyme. Generally, a specific part of the precursor enzyme is cleaved in order to activate it.

For example, a cleavage sequence may be fused to an enzyme to create a zymogen. The cleavage sequence may be the same or similar to that of a substrate that may be present in a biological sample. Cleavage of the sequence by an enzyme will release the inhibition on the zymogen rendering an active proteolytic enzyme. This enzyme would then react with a quantified set of zymogens, which would release a quantified amount of free enzyme, which would react with a quantified amount of specific substrate. For each reaction, a signal may be detected, thereby producing an amplification of a signal. If a biological sample contains a substrate that competes with the cleavage sequence used to create the original zymogen, the signal generated will be reduced.

### Detection of Neuraminidase

In one embodiment, the method can be used to detect neuraminidase activity associated with specific types of bacteria in a biological sample and thus, can also be used to detect bacteria infection. Neuraminidase (also known as sialidase, acylneuraminyl hydrolase, and EC 3.2.1.18) is an enzyme common among animals and a number of microorganisms. It is a glycohydrolase that cleaves terminal alpha-ketosidically linked sialic acids from glycoproteins, glycolipids and oligiosaccharides. Many of the microorganisms, containing neuraminidase on their surface, are pathogenic to man. These pathogenic organisms include bacteria such as Vibrio-Cholerae, Arthrobacter ureafaciens, and bacterial involved in bacterial meningitis, such as Haemophilus influenzae, meningococcal, and pneumococcal meningitis. The meningococcal and some isolates of Haemophilus influenzae express a neuraminidase enzyme that cleaves sialic acid α-2.3 linked to galactose. The meningococcal species recognize cytidine monophospho-N-acetylneuraminic acid (CMP-NANA) and 5-acetylneuraminic acid (Neu5Ac), while Haemophilus influenza recognizes only the Neu5Ac form ((NeuAcα 2-3Gal). The pneumococcus species has been shown to cleave sialic acid-containing substrates with α-2,3 and α-2,6 linkages to galactose as well as those with α-2,6 linkages to N-acetylgalactosamine (NeuAcα 2-3Gal, NeuAcα 2-6Gal, NeuGcα 2-3Gal, NeuGcα 2-6Gal). Other linkages useful for detecting neuraminidase activity include α2-8, α2-9 and cyclic neuraminidic acid linkages. Other linkages useful for detecting neuraminidase activity include α2-8, α2-9 neuraminidic acid linkages (such as meningococcal B and C, and Arthrobacter ureafaciens) and cyclic neuraminidic acid linkages (such as Pseudomonas)

Thus, substrates can be constructed to distinguish neuraminidases originating from different bacterial strains. Examples of sialic acids that can be attached to specific glycoproteins are shown below.

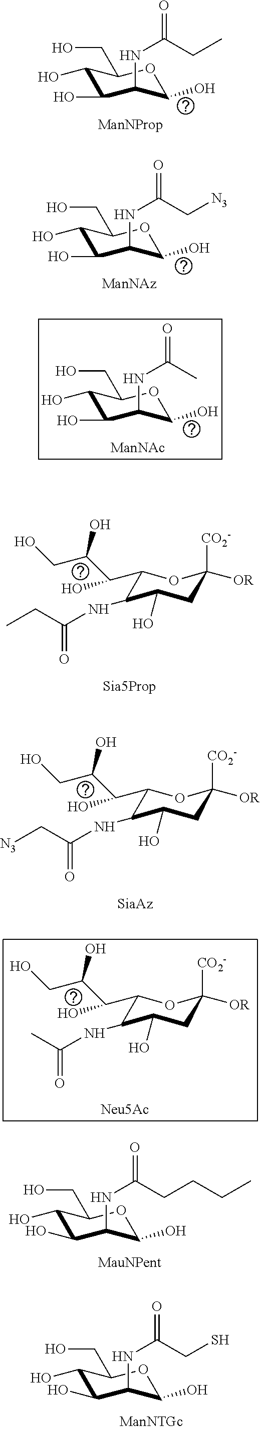

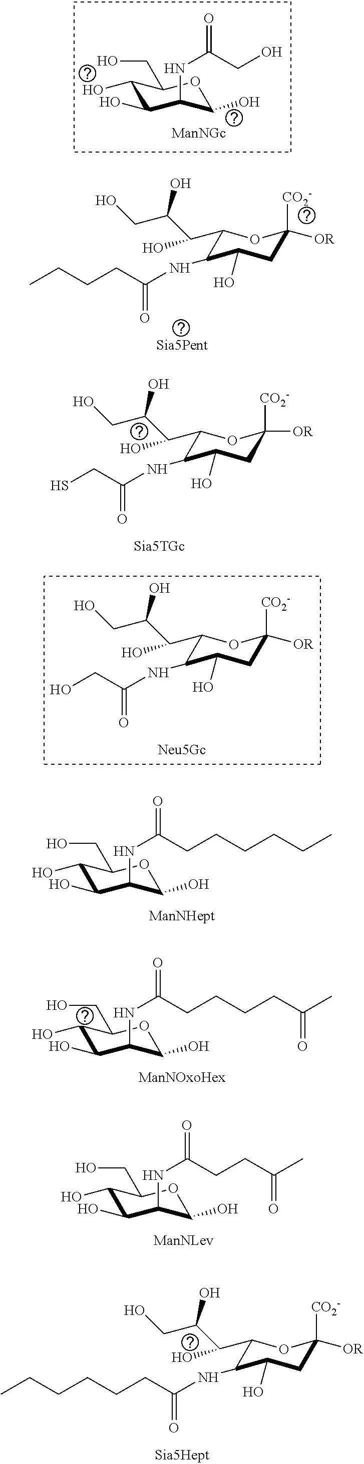

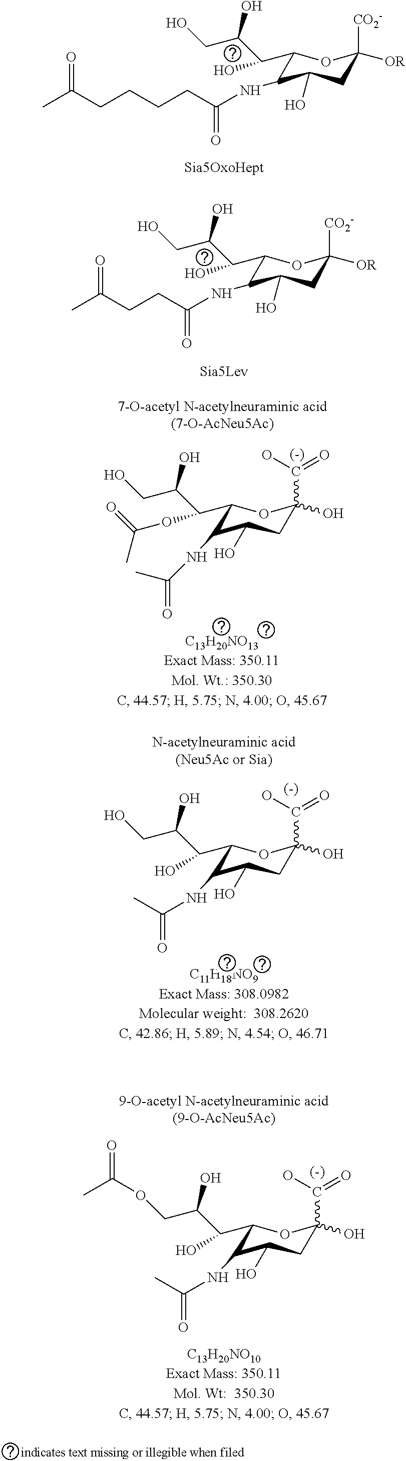

### Detection of Bacterial Meningitis by Detecting Neuraminidase Activity

Detection of the above bacteria in biological samples can be performed by detection of the specific neuraminidases activity. The assay may also be performed in order to detect bacterial meningitis in CSF. As CSF itself has no endogenous neuraminidase activity, the presence of bacterial neuraminidase activity indicates the presence of bacteria in the CSF. Non-limiting examples of such assays for detecting neuraminidase activity in a biological sample are described below.

### Neuraminidase Detection Based on Oligosaccharide Beads and Fluorescence Labeled Ligands (Lectins)

Neuraminidase activity can be detected using sialic acid and its ligand (a lectin). The substrate comprises sialic acid on one end of an oligosaccharide, which is covalently bound to magnetic beads, and a fluorescence-labeled lectin associated with the sialic acid.
SNA—Sambucus Nigra Lectin, specific for Neu5AC(2-6)Gal̂̂.
MAL—Maackia amurensis lectin, Neu5AC(2-3)Gal̂̂.
WGA—Weat Germ aglutinin lectin, most Si-glycan formations.
See FIGS. 14A-14B. Cleavage of this substrate by neuraminidase will result in the separation of the fluorescence-labeled lectin. After the magnetic beads are pulled down, fluorescence can be detected in the supernatant.

Use of NeuAcα 2-3Gal will allow detection of all three meningitis-assosiated bacterial strains. Using NeuAcα 2-6Gal will allow the detection of the pneurnococcu species, and using CMP-NANA will allow detection of the meningococcal species.

In another embodiment, the assay can be performed as a multiplex assay. In this case, the sialic acid-associated oligosaccharides are covalently bound to magnetic beads as described above. The bead/oligosaccharide mixture is first contacted with a biological sample and then the fluorescence labeled lectin is added. If there is no neuraminidase present in the sample, lectin will bind to sialic acid and will be pulled down with the magnetic beads. See FIG. 15, left panel. However, if neuraminidase is present in the sample, the sialic acid will be cleaved from the oligosaccharide and lectin will bind to the sialic acid but will not be pulled down with the magnetic bead. See FIG. 15, right panel.

### Detection of Serotypes

In an embodiment, the method provided can be used to detect serotypes of certain pathogens. In this instance, the enzyme of the pathogen recognizes a common cleavage sequence within the members of the serotype. Alternatively, strains of pathogens may be classified according to similarities in cleavage sequences so that modification of a single substrate represents the pathogens of that class. For example, the Enterovirus (EV) comprises 120 reported human pathogens. The 3C protease is an enzyme in the life cycle of the virus and is relatively conserved between strains. When the proteases and their substrates were analyzed using evolutionary tree analysis and 3D structure modeling between the enzyme and Camb2 substrate [QSY9-Leu-Glu-Ala-Leu-Phe-Gln-Gly-Pro-Pro-Val-Tyr-Cys-(Alexa532)-NH₂]] (SEQ ID NO:59), they could be classified into eight groups (see FIG. 16):
Groups 1 and 6: Enterovirus Group 1 has the largest number of clones (40 out of a total of 73). Most of the clones in group 1 share a identical binding site. Nevertheless, Enterovirus 81, Enterovirus 83 and Echovirus 2 have some irregularities in the composition of the active site accompanied by substitutions to Val and Iso in the cleavage site at the P4 position. However, the presence of this similar cleavage sequences in the rest of the clones in the group indicated that these changes did not affect the ligand-receptor interactions. Therefore, these clones would expect to show similar behavior as Echovirus 30 and Coxsackievirus B5. Comparing group 1 with group 6 indicates substitutions in the margins of the substrate. These substitutions of Phe to Tyr and Leu to Iso are physicochemically conservative and therefore groups 1 and 6 can be gathered into one single group.
Group 2 and 7: Enterovirus Group 2 comprises 4 clones all with identical active sites. However, in the cleavage sequence, there are changes at positions P4 and P5. At position P4, there is either ASN or Thr. This can indicate that either the co-evolved position in the active site is not in the alignment or that P4 has no bearing on the specificity. The difference in the cleavage sequences of groups 2 and 7 is in the margin of the substrate at position P6 having L and M, respectively, These substitutions are conservative substitutions. However, according to preliminary docking results and according to an analysis of the solved structure of the 3C protease of the rhinovirus 14, position P6 of the substrate is found within a conserved patch on the surface of the 3C protease and therefore it may play a role as a specificity determinant.
Group 3: In Enterovirus group 3, all members have identical binding sites and cleavage sequences. Furthermore, positions P3, P4 and P5 are substituted to IEF, respectively, from substrate (Camb 2) (SEQ ID NO:59).
Group 4: Almost all members in this Enterovirus group share the same cleavage site sequence and the same active site. The only aberrant strain is the Coxsackievirus A13, which has in positions P4 and P5, Glu and Phe, respectively, instead of Asn and Phe. Therefore, Coxsackievirus A13 can be assigned either to group 3, which also has Glu and Phe at positions P4 and P5, or to this group. In both cases, the substitutions of the amino acids are physicochemically conservative.
Group 5 and 8: these Enterovirus groups present a unique feature of a charged amino acid in position P4 (Arg or Lys).

Thus, a substrate can be designed such that when it becomes modified by an enzyme, it is indicative of a serotype or subgroup of pathogens.

### Kits

Kits comprising the enzymes or substrates for use in the methods described herein are also provided. In one embodiment, the kit components may be packaged separately and admixed immediately before use. In another embodiment, two or more components may be packaged together. An exemplary kit may comprise one or more of the following reagents: a negative control sample free of an enzyme or substrate; a positive control sample comprising an enzyme or a substrate; a signal generation reagent for development of a detectable signal from the signaling moiety; a sample collection means such as a syringe, throat swab, or other sample collection device; and reagents for performing a separation step. The kits may also comprise an inhibitor and/or an activator of an enzyme.

Packaging of the reagents included in the kits may include, for example, ampules made from glass, organic polymers, ceramic, or metal; bottles; envelopes, test tubes, vials, flasks, syringes, and the like.

Kits may also be supplied with instructional materials. Instructions may be printed on paper or may be supplied in electronic format, such as a floppy disc, CD-ROM, DVD-ROM, etc. Detailed instructions may not be physically associated with the kit; instead, a user may be directed to an internet web site specified by the manufacturer or distributor of the kit, of supplied as electronic mail.

The following examples are intended to illustrate the disclosure without limiting the scope thereof.

### EXAMPLE 1

### Demonstration of Competition Inhibition of Chemotrypsin by Two Substrates

### Reagents and Instruments:

| | | working | | Stock | |
|---|---|---|---|---|---|
| Reagent | description | concentrations | | Con. | |
| a-Chymotrypsin | Enzyme | 100 | ng/ml | 20 | ug/ml |
| Type IV | | | | | |
| Sub.-1 - Camb-2.4 | Substrate | 50 | nM | 1 | mM |

| | | | | |
|---|---|---|---|---|
| Sub.-2 | “Inhibitory” | 40 nM to | 20 | mM |
| M-Suc-Ala-Ala-Pro- | Substrate | 40 uM | | |
| Phe-p-Nitr | | | | |

Dilution and reaction Buffer: HEPES 100 mM pH=7.5.
Substrate 1 (Sub.-1) Camb-2.4 (a batch of Camb2 substrate): FRET substrate based on Alexa 532 fluorophore.
Substrate 2 (Sub.-2): Commercially available colorimetric base (420NM) a-Chymotrypsin substrate. Dissolve in 1:1 HEPES/DMSO to 20 mm. additional 1:10 dilution was done in HEPES buffer.
Enzyme—a-Chymotrypsin: Commercially available a-Chymotrypsin Type IV was diluted in HEPES buffer to a stock of 20 ug/ml. Fluorimeter: DMV, Polar-Star Galaxy. Parameters:
   Ex. 531 mm, Em. 560 mm.
   Temprature RT.
   Total reading Time 10 min. Gain 90

All tests will be performed in triplicates.

### Reaction Procedure

Solution-1: HEPES buffer.
Solution-2: Substrate-1 stock was diluted 1:10 in 1000 ul HEPES to give a 2× solution of 100 nM.
Solution-3, a-d: Substrate-2 stock was serially diluted 1:10 with HEPES and 10 ul of each dilution was added into 40 ul of HEPES. a-400 uM, b-40 uM, c-4 uM, d-400 nM (10× solutions)
Solution-4: Enzyme stock was diluted 1:20 into 300 ul of HEPES to give a 10× solution.

Using a multi-Channel pipette, the above Solutions were added into a non binding blacked bottom 96 wells plat, using columns 1-6 of rows A-C. 30 ul Solution-1 and 50 ul Solution-2 were added to each well. Into rows A-C, 10 ul of Solution-1 was added to columns 1 and 2, 10 ul Solution-3a was added to column 3, 10 ul Solution-3b was added to column 4, 10 ul Solution-3c was added to column 5 and 10 ul Solution-3d was added to column 6. Into rows A-C. 10 ul of Solution-1 was added to columns 1, 10 ul of Solution-4 was added to columns 2-6. Fluorimertic reading was then started.

### Results:

TABLE 3

| | | | | Rate FU/min | Inhibition |
|---|---|---|---|---|---|
| Column | Sub.-1 | Sub.-2 | Enzyme | (Average) | % |
| 1 | 50 nM | 0 | 0 | 0 | — |
| 2 | 50 nM | 0 | 100 pg/ml | 20000 | — |
| 3 | 50 nM | 40 uM | 100 pg/ml | 9000 | 55% |
| 4 | 50 nM | 4 uM | 100 pg/ml | 12000 | 40% |
| 5 | 50 nM | 400 nM | 100 pg/ml | 16000 | 20% |
| 6 | 50 nM | 40 nM | 100 pg/ml | 18000 | 10% |

Under the above conditions, a 10% competitive inhibition can be seen when the two substrates are at about 1:1 in molar ratio. The competition appears to be in a logarithmic scale. Sensitivity can be increased by changing the enzyme concentration, reducing Substrate 1 concentration, and enhancing the fluorimeter gain.

### EXAMPLE 2

### Detection of Procalcitonin (PCT) for the Detection of a Bacterial Infection

The host response to bacterial infection involves the activation of complex immune mechanisms and the release of a wide array of inflammatory mediators. Procalcitonin (PCT) has recently been proposed as a marker of bacterial infection in critically ill subjects. PCT is a 116 amino acid peptide with a sequence identical to that of the prohormone of calcitonin. Muller et al., Crit Care Med (2000) 28:977-83. (FIG. 17). Under normal metabolic conditions, PCT is only present in the C cell of the thyroid gland. However, in bacterial infection and sepsis, intact/truncated (3-116) PCT is found in the blood and, more importantly, its level correlates with the severity of sepsis.

Furthermore, in microbial infections and in various forms of inflammation, circulating levels of several calcitonin precursors, including PCT but not mature calcitonin, increase up to several thousand-fold. This increase and especially the course correlates with the severity of the condition and with mortality. Initially, procalcitonin consisting of 116 amino acids is secreted. Due to rapid cleavage by dipeptidases, a 114 amino acid long procalcitonin is found in the circulation. Additional cleaving leads to circulating aminoprocalcitonin, immature calcitonin and calcitonin carboxypeptide-I (CCP-I), previously known as katacalcin. In sepsis, these peptides are variably increased, often to huge levels due to ubiquitous expression and secretion. However, serum levels of mature calcitonin, which is only produced by thyroidal c-cells, remain normal or are only slightly increased.

Therefore, during sepsis or inflammation, the concentration of procalcitonin in the blood is elevated. In the case of bacterial meningitis, a high correlation was reported between cerebrospinal fluid PCT levels and bacterial meningitis, close to 100%. Hence, quantifying the level of procalcitonin in the blood or even in the CSF would be useful in detecting bacterial infections, such as bacterial meningitis.

In this example, competitive inhibition is utilized for detecting procalcitonin. The method comprises the following compositions:

1) One or more enzymes capable of cleaving PCT, such as prohormone convertase 1 (PC1);

2) One or more substrate compounds capable of being modified by the one or more enzymes capable of cleaving PCT to form corresponding one or more compound derivatives, each of the substrate compounds having the same or different signaling moieties linked to the substrate compounds. In the presence of the enzyme(s), the one or more substrate compound will undergo a modification identical or similar to that which will occur in PCT. For example, the one or more substrates will have a cleavage site identical or similar to the cleavage site of the PCT, with lower affinity to the one or more enzymes.

The assay may be performed with two separate samples: a control sample that does not contain PCT and a test sample that contains a biological sample (e.g. blood or cerebrospinal fluid). The one or more enzymes and the one or more substrate compounds are added to each sample.

If there is no PCT in the test sample, the substrate compounds are modified (e.g. cleaved) at the same rate in both samples. However, if the test sample contains PCT, e.g. the sample contains bacteria, the PCT acts as a competitive substrate for the enzymes that are present in the sample and as a result, a lower overall activity is exhibited from the substrate compounds added to the test sample relative to the control sample.

The values obtained from the assay can also be compared to a clinical reference table. The table, containing ranges of PCT concentrations, may be derived from established clinical data and can be used to correlate the results with severity of bacterial sepsis.

A high PCT concentration test result indicates sepsis and enables the appropriate drug treatment. A low PCT concentration test result indicates normal subject levels and will lead to an appropriate treatment and further testing if applicable.

PCT levels during an infection can be as low as 0.5 ng/ml (40 μM). An amplification system may be beneficial for detecting low levels of infection. Thus, the assay may also comprise an amplification system that amplifies the signal detected from an enzymatic reaction. For example, a cleavage sequence of PCT may be fused to a proenzyme zymogen molecule (for example prothrombin). Cleavage of the PCT sequence by, for example, PC1 or other PCT sequence recognition enzyme will release the inhibition on the proenzyme rendering an active proteolytic enzyme (e.g., thrombin). This enzyme would then react with a quantified set of proenzymes, which would release a quantified amount of free enzyme, which would react with a quantified amount of specific substrate for this enzyme (e.g., thrombin reacting with thrombin substrate).

The assay signal intensity is quantified with different concentrations of enzyme—proenzyme against a control sample that does not contain PCT. Exposing the quantified reaction to different concentrations of PCT, lowers the initial signal intensity. The procalcitonin amount is calculated from the intensity of the final signal. The amplification method allows the detection of serum PCT concentrations as low as 0.5 ng/ml.

Accordingly, examples of substrates that may be used in conjunction with PC1 or other PCT-modifying enzymes include, but are not limited to:
1. A FRET substrate comprising amino acids 8-20 of PCT that includes the PC-1 cleavage site.
2. Amino acids 3-60 of PCT comprising a pNA-colorimetric marker released after PC1 cleavage. This assay is based on spectrophotometric detection of the chromophorep-nitroanilide (pNA) after cleavage from the labeled substrate. The pNA light emission can be quantified using a spectrophotometer or a microtiter plate reader at 400- or 405-nm.
3. Proenzyme: PCT in the biological sample competes with a proenzyme comprising a PC1 specific cleavage sequence. Following cleavage by PC1, the proenzyme becomes activated to modify a substrate comprising a signaling moiety and generates a signal.
4. Full length procalcitonin (116 amino acids)
5. A pro-caspase 3 zymogen, with an activator cap of 4-20 amino acid, that covers the enzyme active site, and contains the sequence GKKR (SEQ ID NO.: 1), or RRKK (SEQ ID NO.: 2) and several other combinations of two adjacent basic amino acid (R&K) therein.

### EXAMPLE 3

### Detection of Methicillin Resistant Staphylococcus Aureus Toxin

The provided method can also be used to detect, for example, community associated methicillin resistant staphylococcus aureus (CA-MRSA) or S. aureus homogeneously resistant to methicillin (HoMRSA) by quantifying the protein levels of specific peptides of MRSA. An example of such a peptide is the 21 aa peptide Phenol soluble modulin (PSM), that is responsible for many disease features of MRSA. Another example is the detection of the Panton-Valentine leucocidin (PVA) toxin that is expressed in higher levels in HoMRSA.

Glutamyl endopeptidase cleaves PSM at the amino acid glu at position 16. To detect PSM and therefore, MRSA, a biological sample is contacted with glutamyl endopeptidase and a substrate comprising a signaling moiety and a glutamyl endopeptidase cleavage site that competes with PSM. The signal from the signaling moiety is measured. It is determined whether there is a difference in the signal compared with a control sample that does not contain PSM. If there is a difference (e.g. a decrease) in signal compared with the control sample, that difference indicates the presence of PSM in the biological sample.

Other exemplary S. Aureus antigens that can be detected according to the provided method include: Ribitol, Polysaccharide A polysaccharide B, Teicoic Acids, Protein A, PVA toxin, PSM toxin, coagulase, staphylokinase, desoxyribonuclease, hyaluronidase, lipase, Hemolysin: alpha, beta, gamma, delta, Valentine Leukocidin, LUK, Exfoliative exotoxin, Toxic Shock Syndrome Toxin, Enterotoxins C,D. Hemolysin gamma, Leukocidin Panton-valentine, TSST-1, Penicillin binding protein 2, and Exfoliative exotoxin.

### EXAMPLE 4

### Substrates for Detection of Fungal Infections

Systemic fungal infections (fungemias) have emerged as causes of morbidity and mortality in immuno-compromised subjects (e.g., AIDS, cancer chemotherapy, organ or bone marrow transplantation). In addition, hospital-related infections in subjects not previously considered at risk (e.g. subjects in an intensive care unit) have become a cause of major health concern. Hence, fungal infections pose a target for diagnosis and therapy.

The method may be utilized for the detection of various fungi, such as, for example, Candida, Cryptococcus neoformans, Aspergillus fumigates, Blastocladiomycota, chytridiomycota, Dikarya, Glomeromycota, and Microsporidia, Neocallimastigomycota.

Candida, which is found in the human digestive tract, mouth, and genital region, is one of the most common organism implicated in fungal infections. Three major extra-cellular hydrolytic enzyme families produced by the Candida species (e.g., C. dubliniensis, C. tropicalis, C. parapsilosis, C. albicans) are the secreted aspartyl proteinases (Sap), phospholipase B enzymes, and lipases.

One specific enzyme is Sap2. Sap2 is expressed abundantly in cultures of Candida albicans and is therefore an attractive target for the detection of Candida infection. The Sap2 cleavage site shows a preference for phenylalanine at the amino acid residue immediately N-terminal to the cleavage sige (“P1” site). A multiple sequence alignment of the substrates of Sap2, including secretory immunoglobulin A, insulin B, albumin, and collagen, provides sequences that may be utilized in the disclosed method. In addition, the alignment has allowed for the construction of artificial substrates that compete with the natural substrate that may be present in a biological sample suspected of containing a fungal infection. The alignment is provided in Table 4 below.

TABLE 4
Substrate compounds cleavable by Sap2.

| P4 | P3 | P2 | P1 | P1′ | P2′ | P3′ | P4′ | Km | SEQ ID NO.: |
|---|---|---|---|---|---|---|---|---|---|
| Arg | Leu | Ile | Gln | Lys | Arg | Ser | Asp | | SEQ ID NO.: 3 |
| Leu | Ile | Gln | Lys | Arg | Ser | Asp | Val | | SEQ ID NO.: 4 |
| — | — | Phe | Val | Asn | Gln | His | Leu | | SEQ ID NO.: 5 |
| Leu | Val | Glu | Ala | Leu | Tyr | Leu | Val | | SEQ ID NO.: 6 |
| Glu | Ala | Leu | Tyr | Leu | Val | Cya | Gly | | SEQ ID NO.: 7 |
| Glu | Arg | Gly | Phe | Phe | Tyr | Thr | Pro | | SEQ ID NO.: 8 |
| Pro | Ala | Leu | Phe | Phe | Arg | Leu | — | 10-15 μM | SEQ ID NO.: 9 |
| Leu | Val | Ile | His | Thr | — | — | — | | SEQ ID NO.: 10 |
| His | Gln | Val | Tyr | Phe | Val | Arg | Lys | | SEQ ID NO.: 11 |
| Pro | Ala | Arg | Phe | Phe | Arg | Leu | — | | SEQ ID NO.: 12 |
| Pro | Ala | Glu | Phe | Phe | Ala | Leu | — | | SEQ ID NO.: 13 |
| Pro | Leu | Glu | Met | Phe | Ala | Leu | — | | SEQ ID NO.: 14 |

### EXAMPLE 5

### Detection of Cancer

A cancerous process alters the expression pattern of genes and more specifically the expression pattern of proteases. These proteases are a part of the intracellular regulation and also of extracellular activities. Extracellular activities include degradation and tissue remodeling of the extracellular matrix that is associated with malignancies, a process mainly facilitated by metalloproteinase.

Several cancer biomarkers have been identified, such as α-fetoprotein (AFP), carcinoembryonic antigen (CEA), prostate-specific antigen (PSA) and CYFRA21. PSA is the most abundant kallikrein-like serine protease in seminal plasma and is measurable in serum after the onset of puberty. The retrograde release of PSA into the bloodstream is a rare event in young healthy men and occurs with a frequency of less than one PSA molecule per million secreted PSA molecules. When the prostate becomes diseased, leakage into blood is highly increased and hence serum PSA serves as a marker of prostate cancer. However, PSA testing in the clinic often suffers from a high false positive rate. Several studies have tried to improve the diagnostic value of PSA through detecting other kallikrain-like proteases, such as KLK2, KLK4 and KLK1. Furthermore, it has been found that in blood, PSA manifests little or no catalytic activity. This is mainly due to a greater than or equal to 105-fold excess of protease inhibitors such as α1-antichymotrypsin (ACT), α2-macroglobulin, and protein C inhibitor (PCI), which inactivate any catalytic PSA by forming stable covalent complexes in serum. Therefore, PSA in blood exists in multiple forms: free or in complexes with the various protease inhibitors. The free form of PSA, which constitutes about 5-35% of the total blood PSA, is catalytically inert. However, PSA photolytic activity can be increased by >103-fold in the presence of 1.3M Na2SO4. Therefore, for determining activity of a member of the kallikerin family, an activator may be added to the biological sample in order to increase the detection of the protease. Alternatively, complexe formation can be reversed by affinity chromatography and can release active PSA. Enzymatic activity of free active PSA may be detected using substrates of PSA that are modified by PSA. Examples of substrates that may be useful for detection of PSA include:

| | |
|---|---|
| HSSKLQ | (SEQ ID NO.: 15) |
| QFYSSN | (SEQ ID NO.: 16) |
| GAGLRLSSYY-SGAG | (SEQ ID NO.: 17) |
| SSIYSQTEEQ | (SEQ ID NO.: 18) |

The substrates further comprise a signaling moiety that produces a signal upon modification, allowing detection of substrates modified by PSA. The signal produced may, for example, be fluorescence.

### EXAMPLE 6

### Detection of the Stroke and Coagulation Cascades Using an Array

An assay for the coagulation cascade can be performed as shown in FIG. 18. Each test tube contains a kinetically calibrated serine protease enzyme of the coagulation cascade at a known concentration and its substrate. The substrate comprises a specific cleavage site and a signaling moiety and retains the kinetic properties of the natural substrate.

A biological sample is added to each test tube. If a substrate of the protease in the test tube is present in the biological sample, it competes with the substrate comprising a signaling moiety and the reaction may be analyzed by FRET substrate based assay or by fluorescence polarization.

The biological sample can be obtained from different populations, and a profile of people at risk for stroke and haematological disorders can be created based on their activities of markers of the coagulation cascade. For example, samples can be obtained from healthy donors, donors at risk for stroke, and donors who have had stroke.

### EXAMPLE 7 Detection of aromatase

Aromatase is a member of the cytochrome P450 superfamily, whose function is to aromatize androgens, producing estrogens. Steroids are composed of four fused rings. Aromatase transforms the left-hand ring (the A-ring) of steroids to an aromatic state (hence the name) through oxidation and subsequent elimination of a methyl group.

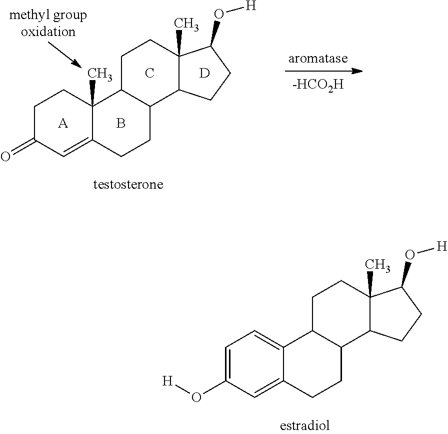

The aromatase enzyme can be found in many tissues including gonads, brain, adipose tissue, placenta, blood vessels, skin, bone, endometrium, breast as well as in tissue of endometriosis, uterine fibroids, breast cancer, and endometrial cancer. While postmenopausal women have low levels of circulating plasma estrogens, the local synthesis or intratumoral production of estrogens that takes place in breast carcinoma tissue itself can lead to higher estrogen levels in the tumor. Thus aromatase inhibitors have become useful in the management of patients with breast cancer whose lesion was found to be estrogen receptor positive.

Aromatase is the final expression of the gonado-endocrine function. Intratumoral aromatase has been considered a viable clinical target for the treatment of estrogen receptor-positive postmenopausal breast cancer patients. However, routine evaluation methods for the detection of aromatase expression in clinical specimens have not been established.

The examination of the localization of aromatase in human tissues may be used as a diagnostic tool for the function of the endocrine system as well as providing better treatment for postmenopausal breast cancer patients by giving information related to the malignancy level.

The method provided can be used to assay different tissues and detect aromatase level as a measure of malignancy and function of the endocrine system in post-menopausal women. An example of a substrate useful for detecting aromatase includes methoxy-4-trifluoromethyl-coumarin (MFC), a fluorogenic substrate that is rapidly converted by aromatase to the highly fluorescent product, 7-hydroxy-4-trifluoromethyl coumarin (7-HFC). Thus, a biological sample from a subject may be obtained and contacted with MFC, and its fluorescence may be measured to detect the presence of aromatase in the biological sample.

### EXAMPLE 8

### Design of Substrates for Detection of Cytomegalovirus (CMV)

Production of Recombinant CMV Protease

CMV encodes a serine protease whose catalytic domain, assemblin (28 kDa), is released by self-cleavage from a 74 kDa precursor (pPR, pUL80a). Assemblin is a serine protease and structural studies revealed that it has a distinctive protein fold and a Ser-His-His catalytic triad. Enzymatic studies have shown that it exhibits allosteric activation through homodimerization. Its dissociation constant is relatively high, approximately 1 μM, but can be decreased about two orders of magnitude by structure-enhancing (kosmotropic) salts, such as Na₂SO₄.

First, a CMV protease was cloned as a source of an enzyme to mimic the endogenous protease produced by CMV. Additional uses of the recombinant protease included use as a positive control, assay validation, optimization, and normalization.

Herpesvirus 5 (CMV), Strain AD-169, ATCC number: VR-538, was purchased from the ATCC as a template for cloning. The forward primer (5′-CACCATGACGATGGACGAGCAG-3′) (SEQ ID NO:60) was added with a 5′ primer extension, CACC (SEQ ID NO:61), to facilitate directional cloning into a topoisomerase cloning vector (pET 151/D-TOPO, Invitrogen) according to the manufacturer's instructions. The reverse primer (5′-TCACGCCTTGACGTATGACTCG -3′) (SEQ ID NO:62) was used to introduce a stop codon, TGA, to the 3′ prime end. PCR was used to amplify the CMV protease gene using a proofreading polymerase (Vent, NEB). PCR products were purified and their integrity was verified by sequencing. They were cloned directly into pET 151/D-TOPO (Invitrogen) and transformed into the E. coli BL21 expression strain.

CMV protease was purified on a Ni—NTA agarose column (QIAGEN, cat. no. 30210) under denaturizing (8M urea) conditions using the 6× His tag conferred on the protease by the expression vector. Purity typically approached approximately 95% by densitometry analysis. Once purified, the CMV protease was refolded by a series of Guanidine HCl dilutions using a dialysis bag with a nominal molecular weight cut-off of 6-8 kDa. After dialysis, the refolded protease was aliquoted in storage buffer (25 mM Hepes, 150 mM NaCl, 1 mM EDTA, 1 mM DTT, 10% Glycerol pH7.5) and stored at −70° C. The concentration of the protease was calculated by fluorescent spectroscopy using the FluoroProfile™ Protein Quantification Kit (Sigma, cat. FP0010).

Substrates

Peptides were designed to be used as competitive substrates for the CMV protease. Peptide sequences were designed to mimic the original cleavage sequence of the CMV protease. HCMV protease cleavage site sequences are known (Baum et al., “Proteolytic activity of human cytomegalovirus u180 protease cleavage site mutants,” J Virol. 1994 June; 68(6): 3742-3752) and are also provided in 5 4 below:

TABLE 5
HCMV protease cleavage site sequences

| P5 | P4 | P3 | P2 | P1 | \\ | P1′ | P2′ | P3′ | SEQ ID NO.: |
|---|---|---|---|---|---|---|---|---|---|
| Gly | Val | Val | Asn | Ala | \\ | Ser | Cys | Arg | SEQ ID NO.: 19 |
| Ser | Tyr | Val | Lys | Ala | \\ | Ser | Val | Ser | SEQ ID NO.: 20 |
| Asp | Asp | Val | Glu | Ala | \\ | Ala | Thr | Ser | SEQ ID NO.: 21 |
| Thr | Ala | Val | Asp | Ala | \\ | Ser | Gly | Asp | SEQ ID NO.: 22 |
| | | Val | Xxx | Ala | \\ | Ser/Ala | | | SEQ ID NO.: 23 |

The HCMV consensus cleavage site deduced from the three known protease cleavage sites in the CMV UL80 polyprotein is VXA, A/S (SEQ ID NO.: 23). See Table 5 above. Examples of useful substrates for detecting a CMV infection also include sequences containing the consensus seqeunce VV (X not K)A-/-S (SEQ ID NO.: 24) or VVNA-/-SCR (SEQ ID NO.: 25). Studies of single-amino-acid substitution mutations within the UL80 cleavage sites confirmed the importance of amino acids in the P3, P1, and P1′ positions relative to the scissile bond.

Specific examples of useful substrates for detecting a CMV infection include the following:

(SEQ ID NO.: 26)
(Dabcy1)-R-G-V-V-N-A-/-S-S-R-L-A-(EDANS)
(herein referred to as Bach1)
(SEQ ID NO.: 27)
QSY9-R-G-V-V-N-A-/-S-S-R-L-A-C(alexa532)-NH2
(herein referred to as Camb3)
(SEQ ID NO.: 27)
QSY9-R-G-V-V-N-A-/-S-S-R-L-A-C(alexa532)-6XPEG-
K(Biotin)-NH2
(herein referred to as Camb4)

The symbol “-/-” denotes a cleavage site by the protease.

Bach1 is commercial peptide from Bachem (lot number 0563789). Peptides Camb3 and Camb4 were synthesized by Cambridge Research Biochemicals Ltd., Cleveland, United Kingdom.

The activity of the recombinant proteases was verified using the substrate, Bach1. Recombinant protease was mixed with Bach1 in a standard assay buffer (25 mM HEPES, 150 mM NaCl, 5 mM EDTA, 5 mM EGTA 5% Glycerol, 0.9 M Na2SO4, 1 mM DTT, pH 8.5). The reaction was monitored using a multi-plate fluoremeter reader (BMG fluostar, Ex. 340 nm, Em. 490 nm). FIG. 19 shows the activity of 200 nM CMV protease with 4 uM of Bach 1.

Substrate Specificity and Cross Reactivity

To determine whether the method is specific for detection of CMV, cross reactivity between human rhinovirus (HRV), Enterovirus (EV) (COX and Echo), SARS and CMV proteases was examined. Reactions were initiated by adding 100 μl of a substrate's stock solution to 100 ul of each protease in the standard assay buffer. 4 μM of the substrates PEP1 (for HRV) [(DABCYL)-L-E-A-L-F-Q-/-G-P-D(EDANS)-S-Q] (SEQ ID NO:63), Elm1 (for EV) [K(DABCYL)L-E-A-L-F-Q-G-P-P-V-Y-E(EDANS)A] (SEQ ID NO:64), PEP6 (for SARS) [(DABCYL)T-S-A-V-L-Q-S-G-F-R-D(EDANS)K] (SEQ ID NO:65), and Bach1 (for CMV) [(DABCYL)-Arg-Gly-Val-Val-Asn-Ser-Ser-Arg-Leu-Ala-(EDANS)] were used and 50 nM, 100 nM, 1.5 uM and 200 nM of HRV, EV, SARS and CMV proteases were used, respectively. Experiments were run in duplicates and repeated twice.

As shown in Table 6, CMV protease was found to be completely specific. CMV protease only cleaved substrates containing a CMV protease cleavage site and not other cleavage sites for other proteases.

TABLE 6

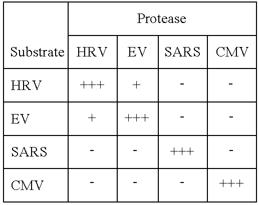

In accordance with the method, at least the following viruses may be detected:
Rhesus cytomegalovirus strain 68-1, Human herpesvirus 5 (strain 1042), Human herpesvirus 5 (strain 119), Human herpesvirus 5 (strain 2387) Human herpesvirus (strain 4654), Human herpesvirus 5 (strain 5035), Human herpesvirus 5 (strain 5040), Human herpesvirus 5 (strain 5160), Human herpesvirus 5 (strain 5508), Human herpesvirus 5 strain AD169, Human herpesvirus 5 strain Eisenhardt, Human herpesvirus 5 strain Merlin, Human herpesvirus 5 strain PT, Human herpesvirus strain Toledo, Human herpesvirus 5 strain Towne, Chimpanzee cytomegalovirus, Aotine herpesvirus 1, Baboon cytomegalovirus OCOM4-37, ercocebus agilis cytomegalovirus 1, Cercopithecus cephus cytomegalovirus 1, Colobus badius cytomegalovirus 1, Colobus guereza cytomegalovirus 1, Crocidura russula cytomegalovirus 1, Macaca fascicularis cytomegalovirus 1, Mandrillus cytomegalovirus, Phacochoerus africanus cytomegalovirus 1, Pongo pygmaeus cytomegalovirus 1, Porcine cytomegalovirus, Simian cytomegalovirus.

### EXAMPLE 9

### Detection of CMV in White Blood Cells (WBC)

CMV infects many cell types including endothelial, epithelial, fibroblast and white blood cells. Therefore, CMV protease may be released into the blood. The first step in detection of CMV protease in blood samples was to work with whole blood. However, no CMV protease activity was detected in whole blood samples. The next step was to work with plasma because it is simple to prepare. The initial testing of plasma samples from CMV subjects did not reveal clear activity. Plasma separated from 60 blood samples were tested and 20 were positive for CMV and 40 were negative. The correlation between the results obtained by the method described below and PCR (performed by Prof. Dana Wolf) was poor. In order to isolate active CMV protease, WBC were isolated from whole blood samples. Surprisingly, when WBC were used, there was a high correlation between the results of the instant method and PCR. Thus, WBC were used for the detection of CMV.

An example of a method for obtaining a sample of white blood cells is provided as follow. 1.5 to 5 ml venous blood is collected into an EDTA-treated tube, using aseptic venipuncture. Blood samples are kept at room temperature (20° C.-25° C., although other temperatures may be used for storage) until processing. Generally, however, processing should be performed within 6 to 8 hours of sample collection in order to avoid WBC lyses. In subjects with severe neutropenia (absolute neutrophil count less than 200/μl), at least 10 ml of blood may be required.

Erythrocyte Lysing Solution (no preservative) is used for the isolation of WBC. A 10× Stock Solution is prepared by dissolving in 1 liter of H₂O: 89.9 g NH4Cl, 10.0 g KHCO3, 370.0 mg tetrasodium EDTA. The pH is adjusted to 7.3. The solution is stored at 4° C. in full, tightly closed 50 ml tubes. A 1× Working Solution is prepared by adding 50 ml of 10× Lysing Stock Solution to 450 ml H₂O and mixed well. It may be stored at room temperature for up to one week.

30 ml Lysing Buffer is mixed with 2 ml blood, is incubated for 5 minutes, and is centrifuged at 1000 rpm for 5 minutes at room temperature. The supernatant is aspirated and the pellet is resuspended in 30 ml of Phosphate Buffered Saline (PBS). The mixture is centrifuged at 1000 rpm for 5 minutes and the supernatant is aspirated and the pellet is resuspended in 1 ml of PBS. The cells are then counted using a hemocytometer or an automated cell counter. The cells are adjusted to a concentration of 1000 cells/μl by diluting in PBS.

A typical in vitro assay for detection of CMV protease is performed at room temperature in standard assay buffer: 25 mM HEPES, 150 mM NaCl, 5 mM EDTA, 5 mM EGTA 5% Glycerol, 0.9M Na2SO4, 1 mM DTT, pH 8.5. A fluorescent labeled substrate is typically used at a concentration of 4 μM. Enzyme concentration may vary from 1 μM to 500 μM.

### Use of Inhibitors

Some clinical specimens may have high levels of background activity that could impair the detection of enzymatic activity This may be due to the presence of unspecific proteases that are capable of cleaving the substrate. Background noise may be reduced by including in the assay one or more inhibitors that can inhibit background activity while having minimal effect on the specific enzyme being tested.

It was found that WBC lysates also had high background activity resulting from non-specific cellular proteases. Based on a comprehensive bioinformatics study, several proteases that can account for background activity were identified. A cocktail of eight inhibitors were chosen to reduce the undesired background: Pestatin A, AEBSF, Aprotinin, E-64, Heparin, Bestatin, GW311616A and eglin C (all inhibitors were obtained from Sigma-Aldrich, except for eglin C, which was obtained from Alexis).

All inhibitors were dissolved at 200X, aliquoted, stored and used according to the manufacturers' instructions. The inhibitors were not freeze thawed. Prior to addition of substrate to initiate the assay, the cocktail was incubated for 2.5 min with the biological sample/CMV protease.

The background activity of the WBC sample was tested by adding 4 μM of the CMV protease substrate Camb4, with or without the inhibitory cocktail. As shown in FIG. 20, the WBC sample without the inhibitory cocktail showed high background noise. Spiking the WBC sample containing the inhibitory cocktail with 50 nM of recombinant CMV protease showed that the inhibitory cocktail exhibited a minor inhibitory effect (up to 20%) CMV protease activity.

Clinical Specimens

Clinical specimen were collected under the Helsinki committee's approval from Soroka Medical Center, Beer sheva, Israel, Hadasa Ein Carem Hospital, Jerusalem, Israel, and Sheba Hospital Tel-Hashomer, Israel. A total of ten blood samples were obtained: 4 from neonates with congenital CMV, 5 from transplanted subjects under treatment for CMV infection, and one from a healthy donor as a control. Specifically, WBC were isolated 6 h post collection, counted, and diluted to 1000 WBC/μl with PBS. 50,000 WBC (50 μl) from each sample was incubated with an inhibitory cocktail (comprising 10 μM Pestatin A, 1 mM AEBSF, 75 μM Aprotinin, 50 μM E-64, 20 U/ml Heparin, 6 μM Bestatin, 10 μM GW311616A and 500 μM eglin C) for 2.5 min. The reaction was started by adding 50 μl of the optimized 1× assay buffer (containing 25 mM HEPES, 150 mM NaCl, 5 mM EDTA, 5 mM EGTA 5% Glycerol, 0.9M Na2SO4, pH 8.5) and 4 μM Camb4 as substrate. Reactions were run in duplicates and measured for 5 mM. Results were compared to the control sample obtained from a healthy individual (sample 10). Samples that had higher signals than those of the healthy individual were considered positive for CMV. All samples were also analyzed for CMV by PCR by Prof. Dana Wolf (samples 1-4) at the virology laboratory at Hadasa Ein Carem Hospital, Jerusalem, Israel, and at Sheba Hospital Tel-Hashomer, Israel, by Prof. Ela Mendelson (samples 5-9). Results are summarized in Table 7.

TABLE 7
Comparison of PCR vs. protease activity test

| Sample | | | | |
|---|---|---|---|---|
| number | Referenee: PCR | | Present Method | Sample origin |
| 1 | 25,000 | copies/ml | High Positive | Congenital CMV |
| 2 | 15,000 | copies/ml | Medium Positive | Congenital CMV |
| 3 | 0 copies/ml Negative | | Negative | Congenital CMV |
| 4 | Weak Positive | | Weak Positive | Congenital CMV |
| 5 | 1700 | copies/ml | Weak Positive | Transplanted subjects |
| 6 | 0 copies/ml Negative | | Weak Positive | Transplanted subjects |
| 7 | 3100 | copies/ml | Weak Positive | Transplanted subjects |
| 8 | <50 | copies/ml | Weak Positive | Transplanted subjects |
| 9 | <50 | copies/ml | Weak Positive | Transplanted subjects |

| | | | |
|---|---|---|---|
| 10 | Negative | Negative | Healthy donor |

Analysis of the results as presented in Table 8 suggests that the instant protease activity test has a positive predictive value of 100% and a negative predictive value of 66%. False positive value was 12.5% and false negative value was 0%. Overall accuracy in these results was 90%.

TABLE 8
Analysis of samples 1-9

Protease activity test

| | | Positive | Negative |
|---|---|---|---|
| PCR | Positive | 7 | 0 |
| | Negative | 1 | 2 |

### EXAMPLE 10

### Substrates for Detection of Retroviruses

The provided method may also be utilized for detecting the following viruses:

Orthoretrovirinae, Lentivirus, Primate lentivirus group

Human immunodeficiency virus 1: HIV-1 M:C_{—}92BR025, HIV-1 M:C_ETH2220, HIV-1 M:F1_BR020, HIV-1 M:F1_VI850, HIV-1 5 M:F2_MP255C, HIV-1 M:F2_MP257C, HIV-1 M:G_{—}92NG083, HIV-1 M:G_SE6165, HIV-1 M:H_{—}90CF056, HIV-1 M:H_VI991, HIV-1 M:J_SE9173, HIV-1 M:J_SE9280, HIV-1 M:K_{—}96CMMP535, HIV-1 M:K_{—}97ZR-EQTB11, HIV-1 N_YBF106, HIV-1 NYBF30, HIV-1 O_ANT70, HIV-1 O_MVP5180, Human immunodeficiency virus 3, Human immunodeficiency virus type 1 (ARV2/SF2 ISOLATE), Human immunodeficiency virus type 1 (BH10 ISOLATE), Human immunodeficiency virus type 1 (BH5 ISOLATE), Human immunodeficiency virus type 1 (BH7 isolate), Human immunodeficiency virus type 1 (BH8 ISOLATE), Human immunodeficiency virus type 1 (BRAIN ISOLATE), Human immunodeficiency virus type 1 (BRU ISOLATE), 15 Human immunodeficiency virus type 1 (CDC-451 ISOLATE), Human immunodeficiency virus type 1 (CLONE 12), Human immunodeficiency virus type 1 (ELI ISOLATE), Human immunodeficiency virus type 1 (HXB2 ISOLATE), Human immunodeficiency virus type 1 (HXB3 ISOLATE), Human immunodeficiency virus type 1 (isolate YU2), Human immunodeficiency virus type 1 (JH3 ISOLATE), Human immunodeficiency virus type 1 (JRCSF ISOLATE), Human immunodeficiency virus type 1 (KB-1 isolate), Human immunodeficiency virus type 1 (Lai isolate), Human immunodeficiency virus type 1 (MAL ISOLATE), Human immunodeficiency virus type 1 (MFA ISOLATE), Human immunodeficiency virus type 1 (MN ISOLATE), Human immunodeficiency virus type 1 (NDK ISOLATE), Human immunodeficiency virus type 1 (NEW YORK-5 ISOLATE), Human immunodeficiency virus type 1 (NIT-A isolate), Human immunodeficiency virus type 1 (OYI ISOLATE), Human immunodeficiency virus type 1 (PV22 ISOLATE), Human immunodeficiency virus type 1 (RF/HAT ISOLATE), Human immunodeficiency virus type 1 (SC ISOLATE), Human immunodeficiency virus type 1 (SF162 ISOLATE), Human immunodeficiency virus type 1 (SF33 ISOLATE), Human immunodeficiency virus type 1 (STRAIN UGANDAN/ISOLATE U455), Human immunodeficiency virus type 1 (WMJ1 isolate), Human immunodeficiency virus type 1 (WMJ2 ISOLATE), Human immunodeficiency virus type 1 (Z-84 ISOLATE), Human immunodeficiency virus type 1 (Z2/CDC-Z34 ISOLATE), Human immunodeficiency virus type 1 (ZAIRE 3 ISOLATE), Human immunodeficiency virus type 1 (ZAIRE 6 ISOLATE), Human immunodeficiency virus type 1 (ZAIRE HZ321 ISOLATE), Human immunodeficiency virus type 1 1w12.3 isolate. Human immunodeficiency virus 2:Human immunodeficiency 5 virus type 2 (ISOLATE BEN), Human immunodeficiency virus type 2 (ISOLATE ROD), Human immunodeficiency virus type 2 (ISOLATE ST), Human immunodeficiency virus type 2 (isolate ST/24.1C#2), HIV-2 BEHO, HIV-2 B_UC1, HIV-2.D205, Human immunodeficiency virus type 2 (ISOLATE D205,7), Human immunodeficiency virus type 2 (isolate 7312A), Human immunodeficiency virus type 2 (ISOLATE CAM2), Human immunodeficiency virus type 2 (ISOLATE D194), Human immunodeficiency virus type 2 (ISOLATE GHANA-1), Human immunodeficiency virus type 2 (isolate KR), Human immunodeficiency virus type 2 (ISOLATE NIH-Z), Human immunodeficiency virus type 2 (ISOLATE SBLISY).

For the detection of the above viruses, a competitive substrate having the following formula can be used:

(SEQ ID NO.: 28)
(S/G)(Q/G/R/K)(N/C/D)(Y/Hydrophobic/Aromatic)-/-P(I/V/Hydrophobic)(V/Q)

Other viruses to be detected may include, without being limited thereto:

Retroviridae; Orthoretrovirinae; Deltaretrovirus; Primate T-lymphotropic Human T-cell lymphotrophic virus type 1 (Caribbean isolate), Human T-cell lymphotrophic virus type 1 (isolate MT-2), Human T-cell lymphotrophic virus type 1 (strain ATK), Human T-cell lymphotropic virus type 1 (african isolate), Human T-cell lymphotropic virus type 1 (north american isolate).

HTLV (Human T-Cell Lymphotrophic Virus)

Peptides that can be used as competitive substrates for the detection of HTLV may include a cleavage site at the capsid and nucleocapsid (CA/NC) and Pr/P3. These include, without being limited thereto:

(SEQ ID NO.: 29)
(V/L/T/P)X(Hydrophobic)(F/L) -/- V(Hydrophobic)Q
(SEQ ID NO.: 30)
KVKV(F/L) -/- VVQPK
(SEQ ID NO.: 31)
PPX(Hydrophobic)L -/- PI

### EXAMPLE 11

### Neuraminidase Activity Detection Based on Oligosaccharide Beads and Fluorescent Labeled Ligands (Lectins)

Determining whether the cause of meningitis is viral or bacterial may improve the nature and course of treatment of patients suspected of having meningitis infection. Because bacterial strains causing meningitis are associated with extracellular neuraminidase activity and not in the CSF, monitoring neuraminidase activity in the serum may be used as an indicator of bacterial meningitis infection. Uses of the NeuAcα 2-3Gal neuraminidase system allows the detection of all three meningitis associated bacteria strains.

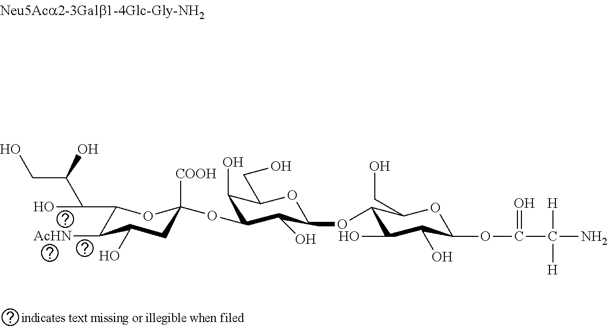

### Reagents:

Si-NeuAcα 2-3Gal, MW=600 dalton (Dextra Laboratories UK, Cat. # NH312)
6-Joe-SE (6-Carboxy-4′,5′-dichloro-2′,7′-dimethoxyfluorescein, succinimidyl ester): MW=600 dalton (AnaSpec USA, cat. #81011)
Lectins: MAL—Maackia amurensis lectin, Neu5AC(2-3)Gal̂̂. MW=150000 daltons. (Sigma, Cat.#L8025)
Beads—M280/MyOne-dynabeads-tos. Activated. (Invitrogen)
Neuraminidase enzyme: a(2-3)-NNeuraminidase from Streptococcus pneumoniae (Sigma, Cat. #N7271).
All other salts were purchased from Sigma-Aldrich.

### Si-Sugar/Dynabeads-Tos.

0.1 M sodium Borat, pH 9.5 (labeling buffer A)
3M Ammonium Sulfate in 0.1 M sodium Borate, pH 9.5 (labeling buffer B)
HEPES buffer (35 mM) pH=7.4, contains MnCl₂ 10 mM and CaCl₂ at 20 mM) Binding Buffer)
0.1 M Tris-HCl pH=9.0
1). Incubate 50 ul pellet beads (from 500 ul reagent, capacity for about 250 nm) with 100 ul of 5 mg/ml (0.5 mg, 750 nm) of NH₂-NeuAcα 2-3Gal-Si, 330 ul Buffer-B and 520 ul buffer-A, for 36 h at room temperature in a 2 ml round bottom tube with rolling.
2). Add 1 ml 0.1M Tris and incubate overnight at room temperature.
3). Wash 5× with 1.0 ml Binding Buffer and resuspend in 1500 ul Binding buffer.

### Lectins-Joe (SE-NH2 Reaction)

Prepare 1.0 M Tris-HCl pH=8.0
Prepare 0.1 M sodium tetraborate, pH 8.5 (labeling buffer C)
1). Dissolve 1000 μg (1.6 uM) of the 6-Joe-SE in 60 μL DMSO.
2). Transfer 5 ul of #1 to a round bottom tube and add 200 ul of 5 mg/ml MAL in buffer C (1 mg, 0.0065 um-1:20 m:m with Joe)
3). Incubate overnight at room temperature with rolling motion.
6). Add 30 ul of Tris 1.0M and incubate overnight at room temperature.
7). Load 250 ul onto a dry 5 ml Sephadex G-25 column (calibrated to binding buffer) and collect supernatant. Store at 4° C.

### Preparation of Binding of Lectins to Beads-Si

Pull down 750 ul magnetic beads in a round dark 2 ml tube and resuspend in 500 ul binding buffer. Add 100 ul labled lectin. Incubate 6 h at room temperature with rolling motion (under aluminum paper). Wash 7× with binding buffer 1 mg/ml BSA and resuspend in 2000 ul binding buffer.

### Enzymatic Reaction

50 ul of lectin-associated beads were incubated for 15 min at room temperature with or without 1U of the a-(2-3)-Neuraminidase (positionally specific from Streptococcus pneumonia). Magnetic beads were pulled down and fluorescence was measured.

### Results

Release of the lectins from beads by one of the Streptococcus pneumonia neuraminidases is shown in Table 9 below.

TABLE 9

| | Buffer | (2-3) Enz |
|---|---|---|
| | % release | % release |
| Beads-a(2-3)-Si/MAL-Joe | 5 | 35 |

These results indicate that neuraminidase treatment of the lectin associated beads, by a free enzyme, has the ability to cleave sialic acid from the sugar backbone, causinthe release of the lectin from the beads.

Pretreatment of Beads-Si with Neuraminidase before Lectin Binding

100 ul of oligosaccharide-labeled beads were incubated for 15 min at room temperature with 1U of the a-(2-3)-Neuraminidase. Beads were pulled down and resuspended in 200 ul of binding buffer and 10 ul of the relevant fluorescence labeled lectin was added. After incubation for 2.5 h at room temperature with rolling motion, the beads were washed 5× with binding buffer and measured for fluorescence.

Table 10 shows the results of % lectin binding to treated beads.

TABLE 10

| | Buffer | (2-3) Enz |
|---|---|---|
| | % bound | % bound |
| Beads-a(2-3)-Si/MAL-Joe | 40 | 5 |

These results indicate that pretreatment of the Bead-Si with neuraminidase highly reduced the association between the lectins and the beads.

### EXAMPLE 12

### Inhibitors of Non-Specific Protease Activity

Inhibitors against non-specific proteases that may interfere with the provided method may be useful for reducing background noise and increasing the precision of detection of a pathogen, disease, or medical condition, or biomarker thereof, from a biological sample. A bioinformatics study was first performed to identify non-specific proteases in each tissue/organ and inhibitors that may be effective against those proteases.

The bioinformatics research was conducted according to following stages:
1) In this first stage, all the proteases that are known today were searched in the literature. The protease search was conducted through a Medline literature search (and the articles it cited) and Medline Nucleotide search. Redundancies in protease names were eliminated. The search resulted in 442 proteases.
2) In this stage, every known protease was associated with tissues in which it is expressed. Every protease was searched against the following protein databases:
   a. Medline.
   b. http://www.hprd.org
   c. http://www.brenda.uni-koeln.de

Those databases were also used to categorize every protease according to its catalytic active site (Serine, Cysteine, Aspartic, metalloprotease, or Ubiquitin proteasome system protein).

Each tissue was attributed to the biological system or organ in question. A visual basic program was written into an excel based database. The application enables the user to mark the tissues of interest. Once the user marks the tissues of interest, the proteases that are expressed in these tissues are shown. The following is a list of the tissues analyzed:

| | | |
|---|---|---|
| Lymphocyte | Antrum | CNS |
| Epididymis | Tongue | Eye |
| Oesophagus | Caecum | Lung |
| Ubiquitous | Jejunum | Milk |
| Neutrophil | Myocyte | Pons |
| Hepatocyte | Stomach | Bone |
| Lymph node | Trachea | Iris |
| Th(2) cell | Gingiva | Lens |
| Cerebellum | Decidua | Aorta |
| Endocervix | Putamen | Ovary |
| Melanocyte | Urethra | Ileum |
| Enterocyte | Chorion | Colon |
| Th(1) cell | Pharynx | Semen |
| Glomerulus | Cochlea | Serum |
| Eosinophil | Prostate | Brain |
| Urothelium | Pancreas | Fetus |
| Myocardium | Platelet | Liver |
| Spermatozoa | Placenta | Heart |
| Bone marrow | Cerebrum | Liver |
| Leydig cell | Midbrain | Blood |
| Odontoclast | Monocyte | Urine |
| Hippocampus | Bronchus | Serum |
| Endometrium | Duodenum | Tooth |
| Spinal cord | Thalamus | Heart |
| Grey matter | Amygdala | Testis |
| Parathyroid | Basophil | Kidney |
| Goblet cell | Intestine | Uterus |
| Endometrium | Cartilage | Cornea |
| Chondrocyte | Microglia | Plasma |
| Granulocyte | CD8+ cell | Vagina |
| Trophoblast | Leukocyte | Rectum |
| Spermatocyte | Epidermis | Tonsil |
| Keratinocyte | Ventricle | Spleen |
| Muscle fibre | Thymocye | Thymus |
| Blood vessel | Astrocyte | Cervix |
| Ciliary body | Bile duct | Embryo |
| Gall bladder | Neuroglia | Atrium |
| Frontal lobe | CD4+ cell | BCell |
| White matter | Macroglia | Muscle |
| Schwann cell | Mast cell | Retina |
| Vas deferens | Macrophage | T Cell |
| Hypothalamus | | Artery |
| Renal tubule | | Neuron |
| Pineal gland | | |
| Sertoli cell | | |
| Renal cortex | | |
| Nasal mucosa | | |
| Thyroid gland | | |
| Mammary gland | | |
| Megakaryocyte | | |
| Temporal lobe | | |
| Adrenal gland | | |
| Loop of Henle | | |
| Smooth muscle | | |
| Spermatogonia | | |
| Purkinje cell | | |
| Atrial septum | | |
| Renal medulla | | |
| Parietal lobe | | |
| Gastric mucosa | | |
| Adrenal cortex | | |
| Nervous system | | |
| Vitreous Humor | | |
| Red blood cell | | |
| Occipital lobe | | |
| Dendritic cell | | |
| Salivary gland | | |
| Fallopian tube | | |
| Choroid plexus | | |
| Ependymal cell | | |
| Amniotic fluid | | |
| Lamina propria | | |
| Adipose tissue | | |
| Olfactory bulb | | |
| Cardiac muscle | | |
| Cervical mucus | | |
| Umbilical cord | | |
| Skin fibroblast | | |
| Adrenal medulla | | |
| Small intestine | | |
| Lung epithelium | | |
| Cerebral cortex | | |
| Urinary bladder | | |
| Cerebral neuron | | |
| Skeletal muscle | | |
| Dentate nucleus | | |
| Stratum corneum | | |
| Collecting duct | | |
| Pituitary gland | | |
| Coronary artery | | |
| Corpus callosum | | |
| Cytotrophoblast | | |
| Caudate nucleus | | |
| Seminal vesicle | | |
| Large intestine | | |
| Adenohypophysis | | |
| Oligodendrocyte | | |
| Sebaceous gland | | |
| Chromaffin cell | | |
| Germ cell layer | | |
| Ascending colon | | |
| Small inetstine | | |
| Substantia nigra | | |
| Pulmonary artery | | |
| Vascular endothelium | | |
| Islets of Langerhans | | |
| Mononuclear phagocyte | | |
| Intestinal epithelium | | |
| Peripheral blood cell | | |
| Granulosa lutein cell | | |
| Gastrointestinal Tract | | |
| Corneal epithelial cell | | |
| Hematopoietic stem cell | | |
| Gall bladder epithelium | | |
| Distal convoluted tubule | | |
| Ovarian follicular fluid | | |
| Bronchial epithelial cell | | |
| Vascular endothelial cell | | |
| Pancreatic duct epithelium | | |
| Proximal convoluted tubule | | |
| Proximal tubular epithelium | | |
| Umbilical vein endothelial cell | | |
| Retinal pigmented epithelial cell | | |
| Cerebrospinal fluid | | |
| Corneal endothelium | | |
| Syncytiotrophoblast | | |
| Zona fasciculata | | |
| Transverse colon | | |
| Descending colon | | |
| Ovarian follicle | | |
| Chorionic villus | | |
| Synovial membrane | | |
| Medulla oblongata | | |
| Pancreatic acinus | | |
| Cerebellar cortex | | |
| Myeloid stem cell | | |
| Paracentral gyrus | | |
| Nucleus accumbens | | |
| Submaxillary gland | | |
| Vermiform appendix | | |
| Mammary epithelium | | |
| Ventricular septum | | |
| Placental membrane | | |
| Uterine myometrium | | |
| Ciliary epithelium | | |
| Natural killer cell | | |
| Alveolar macrophage | | |
| Subthalamic nucleus | | |

3) Using the previously described tissue selecting application, proteases that are expressed in the respiratory system were identified. The tissues that were chosen are:

| | |
|---|---|
| Submandibular gland | Lungs |
| Saliva | salivary gland |
| Bronchus | Tongue |
| Alveolar macrophage | Nasal mucosa |
| Tooth | Lung epithelium |
| Ciliary epithelium | Trachea |
| Pharynx | Bronchial epithelial cell |
| Tongue | Gingival |
| Leukocyte | Nasal mucosa |
| macrophages and granulocyte | neutrophils |

The selection of these tissues marked the following 72 proteases as being expressed in the respiratory system:

| | |
|---|---|
| tryptase alpha | Renin |
| Dehydropeptidase | Hepsin |
| Tryptase epsilon | Napsin-1 |
| Renal dipeptidase | Testisin |
| rine protease EOS | Calpain 7 |
| Serine protease 15 | Caspase 4 |
| Serine protease 25 | Caspase 5 |
| Serine protease 8 | Caspase 7 |
| Cysteine protease 2 | Caspase 9 |
| protease serine 11 | Caspase 10 |
| elastase, leukocyte | Granzyme K |
| Placental protein 11 | Kallikrein |
| Serine protease, HTRA3 | Matriptase |
| Dipeptidyl peptidase IV | cathepsin E |
| ClpP caseinolytic protease | Cathepsin B |
| Tripeptidyl aminopeptidase | Cathepsin C |
| Airway trypsin like protease | Cathepsin G |
| ClpX caseinolytic protease X | Cathepsin H |
| Mosaic serine protease-corin | Cathepsin K |
| Beta site App cleaving enzyme | Cathepsin S |
| Ubiquitin specific protease 35 | Cathepsin Z |
| Beta site APP cleaving enzyme 2 | Pancreasin |
| Plasminogen activator urokinase | Proteinase 3 |
| Transmembrane serine protease 2 | Cathepsin A |
| Signal peptide peptidase-like 2B | Cathepsin O |
| O-sialoglycoprotein endopeptidase | Kallikrein 4 |
| Pigment epithelium derived factor | Kallikrein 5 |
| Squamous cell carcinoma antigen 2 | Kallikrein 7 |
| Stratum corneum chymotryptic | Kallikrein 8 |
| enzyme | Kallikrein 9 |
| Protein convertase subtilisin/kexin | Kallikrein B |
| type 5 | Kallikrein 10 |
| Mucosa associated lymphoid tissue | Kallikrein 11 |
| lymphoma translocation protein 1 | Kallikrein 12 |
| | Kallikrein 13 |
| | Kallikrein 15 |
| | Kallikrein 6 |
| | Trypsinogen 2 |
| | all Kallikrein |
| | Cathepsin F |
| | Tryptase beta 1 |

4) For every protease, information was gathered regarding its cleavage site preferences and its known inhibitors. During this process we used literature and the following databases:
   a. http://www.brenda.uni-koeln.de
   b. www.hprd.org
   c. http://www.ebi.uniprot.org/index.shtml
   d. http://www.peptide.co.jp.
   e. http://merops.sanger.ac.uk
   f. www.expasy.org
5) The cleavage profiles of the identified proteases were used to determine which of the proteases might cleave the human Rhinovirus (HRV) peptide, pep9 (DABCYL)-L-E-A-L-F-Q-P-D(EDANS)-S-Q-NH₂ (SEQ ID NO.: 32). Proteases for which there are no known cleavage site were considered proteases that might also cleave pep9. The non-specific metalloproteases are strongly inhibited by EDTA, which can be added to almost all samples, and therefore were not considered in this analysis.

The proteases that are capable of cleaving pep9 include:
Tryptase epsilon
Serine proteasEOS
Serine protease 15
Serine protease 25
Serine protease 8
Placental protein 11
ClpP caseinolytic protease
Tripeptidyl aaminopeptidase
Airway trypsin like protease
Mosaic serine protease-corin
Ubiquitin specific protease 35
Pigment epithelium derived factor
Paracaspase
   Cathepsin A
   Cathepsin O
   Kallikrein 5
   Kallikrein 8
   Kallikrein 9
   Kallikrein B
   Kallikrein 10
   Kallikrein 11
   Kallikrein 12
   Kallikrein 13
   Kallikrein 6
   Proteinase 3
   Kallikrein 15
   Cathepsin F
   Renin
   Protease
   Napsin-1
   Calpain 7
   Caspase 4
   Caspase 5
   Caspase 7
   Caspase 9
   Caspase 10
   Elastases
   Cathepsin B
   Cathepsin H
   Cathepsin K
   Pancreasin
   cathepsin E
6) After the proteases most likely to cleave the peptide pep9 have been identified, the smallest group of protease inhibitors that could inhibit these proteases were identified. The literature was reviewed for the inhibitory effect on the proteases, toxicity, and availability of the inhibitors in step 4. Another consideration was the known effect of the inhibitors on HRV 3C according to Wang et al., Anal. Biochem. 252:238-245 (1997).
7) As can be seen in Table 11 below, there are about 40 proteases that may need to be inhibited to reduce non-specific protease activity and thus, background noise. Based on a review of the literature, it has been found that 5 inhibitors should inhibit these proteases and lower the background reading of the assay in fluid from the respiratory system without lowering the HRV 3C protease activity. In Table 8, EDTA was added for its inhibitory effect on the metalloproteases.

TABLE 11

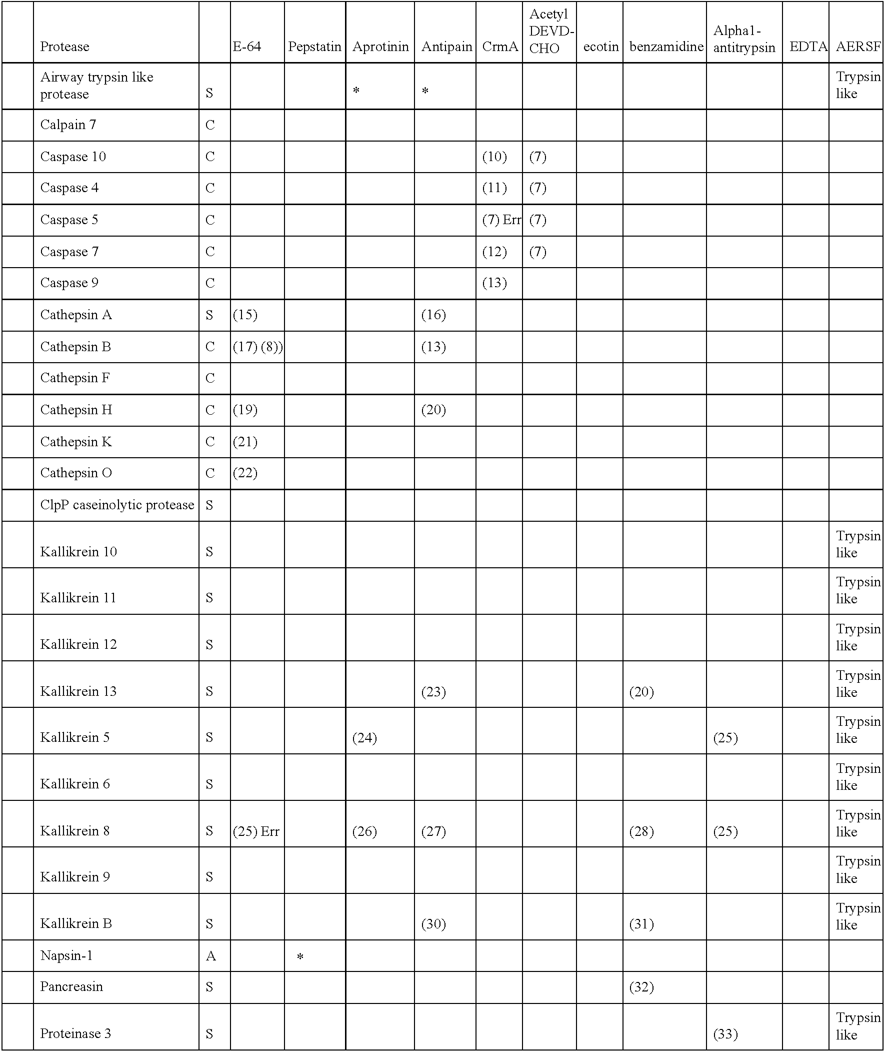

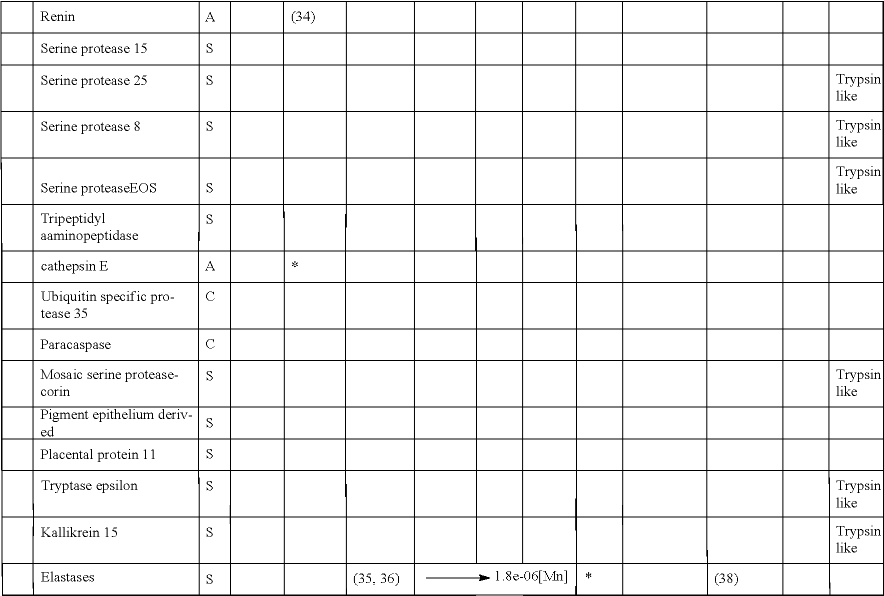

Legend for Table 11.

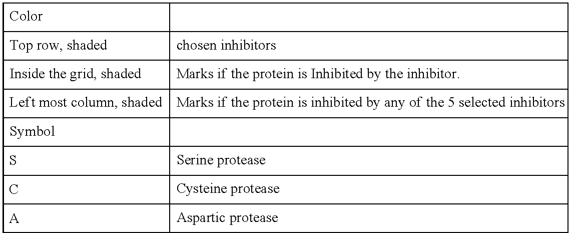

### EXAMPLE 13

### Nasal Wash Inhibitory Cocktail for Detection of HRV

Using the information gathered in the previous example, seven inhibitors that theoretically should abolish non specific proteases activities were chosen. These inhibitors include: Pestatin A, AEBSF, Aprotinin, E-64, Ac-DEVD-CHO, EDTA+EGTA and Eglin C. Bestatin was also selected as it is a useful aminopeptidase inhibitor. These inhibitors were tested for their activity against background noise and HRV protease specific activity.

All inhibitors were dissolved at 100×, aliquoted, stored and used according to the manufacturers' instructions. No freeze thaw cycles were imposed on the inhibitors. Prior to starting the assay by addition of substrate, 10 μl of the cocktail or individual inhibitor was incubated for 5 min with 90 μl of the sample/buffer+ HRV protease.

To test the effect of the inhibitors on protease activity (Buffer+3C) and on background noise present in a nasal wash specimen pool, reaction rates were compared with or without the inhibitors in question (Table 12). Reactions were performed at 4 μM PEP1 and 50 nM recombinant HRV 3C protease for 5 min under starting buffer environment. A nasal wash specimen pool of four HRV negative specimens by RT-PCR was used due to insufficient volume from an individual specimen and to reproduce equal conditions throughout the experiments.

Base on these results (Table 12), the final nasal wash inhibitory cocktail was chosen: 1 mM AEBSF, 7.5 μM Aprotinin, 5 mM EDTA+EGTA each (normally introduced by the assay buffer), 50 μM E-64 and 0.5 μM Eglin C. Pepstatin A, Ac-DEVD-CHO and Bestatin were omitted since they had no effect on the non specific proteolytic activity of the nasal wash specimen (Table 12). The inhibitor E-64 was included due to its potent cysteine proteases inhibition potential and it showed no inhibition against HRV 3C protease activity (Table 12).

TABLE 12

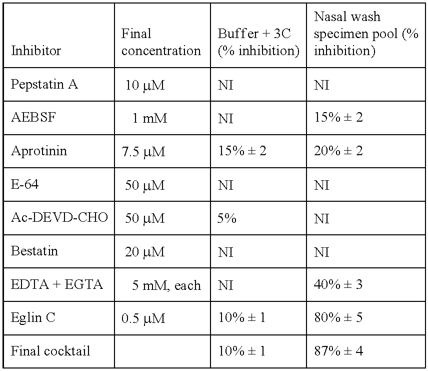

NI: No Inhibition; (±): SD of duplicates.

The inhibitor cocktail was also tested against a specimen pool and WBC lysate with or without the cocktail and with spiked 50 nM recombinant HRV 3C protease. See FIGS. 21A-B. WBCs are abundant in inflammatory nasal wash and therefore, WBC lysates were tested. WBC lysate had high background activity in the HRV assay (with the substrate PEP1) resulting from non-specific cellular proteases. It was assumed that part of the background observed in the specimen pool originated from WBC.

Results with and without inhibitory cocktail and with cocktail+recombinant HRV 3C protease are shown for both the specimen pool (FIG. 21A) and human WBC at ˜1×10⁶ cell/ml (FIG. 21B). The cocktail inhibited background activity in both the specimen pool and WBC lysate with high potency (>87%). Spiking of recombinant HRV 3C protease to reactions with the inhibitory cocktail induced specific HRV protease activities at normal rates. Reactions were performed with 4 μM PEP1 and 50 nM recombinant HRV 3C protease where indicated (by +3C). The data are representative of two independent repeats.

To further improve the inhibitory cocktail and broaden the non specific proteases it covers, four additional inhibitors were added: Bestatin, Pepstatin A, Heparin and GW311616A. These inhibitors did not inhibit the HRV 3C recombinant protease and based on their known target proteases, they may be relevant for other human body fluids specimens. After the addition of these four inhibitors, the new inhibitory cocktail named I8 was established. See Table 13.

TABLE 13

| | | | final concentration |
|---|---|---|---|
| Name | Type | known target proteases for inhibition | used (uM) |
| AEBSF | serine protease | trypsin, chymotrypsin, plasmin, | 1000 |
| | inhibitor. | kallikrein, and thrombin | |
| Aprotinin | competitive serine | Acrosin, Chymotrypsin, | 7.5 |
| | protease | Chymotrypsinogen, Elastase, | |
| | | Kallikrein, Plasmin, Plasminogen | |
| Bestatin | synthetic dipeptide | leucine aminopeptidase, | 20 |
| | | aminopeptidase B, and triamino | |
| | | peptidase | |
| E-64 | irreversible cysteine | Actinidin, Ananain, Bromelain, | 50 |
| | protease | Calpain, Cathepsin B, Cathepsin B1, | |
| | | Cathepsin H, Cathepsin L, Camepsin, | |
| | | Clostripain, Comosain, CMP-Sialic | |
| | | Acid: Lactosylceramide, α(2-3)- | |
| | | Sialytransferase, Ficin, α-Ginivain, | |
| | | Papain, α-and β-Trypsin | |
| Eglin C | 70 amino acid peptide | chymotrypsin, subtilisin, leukocyte, | 0.5 |
| | | elastase and cathepsin G. | |
| GW | | human neutrophil elastase | 10 |
| Pepstatin A | aspartyl proteases | pepsin, renin, cathepsin D, chymosin, | 10 |
| | | prorease B | |
| Heparin | mucopolysaccharide or | human elastase | 2.00 |
| | a glycosoaminoglycan. | | |

### EXAMPLE 14

### Inhibitory Cocktail for Detection of Enteroviruses in Cerebrospinal Fluid (CSF)

Methods and compositions for detecting meningitis has been disclosed in WO 2007/029262. However, CSF (Cerebrospinal fluid) samples used for detecting enterovirus with the enterovirus substrate Camb2 generate background activity. To identify inhibitors that could be used to reduce background noise, non-specific proteases that may be present in CSF and their inhibitors were identified using the method described in Example 12.

### Activity, Design and Results:

1) To find all the proteases that are expressed in tissues that come in contact with the CSF, a literature search and a database search were conducted. The databases that were used included: MEROPS, BRENDA, OMIM, NCBI and HPA. This search included the following tissues: Lymphocyte, Brain, Leukocyte, Nervous system, Adrenal medulla, Adrenal cortex, Cerebral cortex, Cerebral neuron, CNS, Spinal cord, T Cell, Substantia nigra, Cerebrum, Macrophage, Hippocampus, B Cell, Neutrophil, Cerebellum, Medulla oblongata, Frontal lobe, Temporal lobe, Thalamus, Amygdala, Grey matter, White matter, Schwann cell, Pituitary gland, Cerebrospinal fluid, Neuroglia, Parathyroid, Hypothalamus, Zona fasciculata, Cerebellar cortex, Pineal gland, Neuron, Eosinophil, Purkinje cell, Granulocyte, Basophile.
2) This search produced 227 proteases. According to their sequence homology and according to the literature, the proteases were divided into three categories:
   a. Metalloproteases (39 members).
   b. ATP-dependent proteases (41 members).
   c. Serine/Cysteine and aspartic proteases (147 members).
3) The expected concentration of ATP in the specimen reduces the probability that an ATP-dependent protease would cleave the substrate. Furthermore, the use of a chelator (EDTA) in the reaction buffer inhibits the activity of the non-specific metalloproteases. Therefore, after the elimination of the metalloproteases and the ATP-dependent proteases as possible non-specific cleaving agents, the serine/cysteine and aspartic proteases were candidates for non-specific protease activity in CSF.
4) For each serine/cysteine and aspartic proteases, comprehensive literature and database searches were performed for their cleavage site preferences. A number of these proteases were eliminated because their cleavage sites showed that they would not cleave the substrate of interest, Camb2. This elimination process reduced the probable proteases from 227 to 51 proteases.
5) For the remaining 51 proteases, an additional literature search, to minimize (as much as possible) the number of inhibitors used in the cocktail, was conducted for their protease inhibitors (and in some cases inhibitor intra-interactions), pH dependency of the inhibitors and the proteases, incubation time for the protease/inhibitor, and whether the inhibitor is commercially available.
6) The proposed inhibitors useful for detecting enterovirus in CSF samples using Camb2 are listed below:
Granzyme H, Proteinase 3: α-1-Proteinase inhibitor.
Dipeptidyl peptidase: Diprotin A, L-2,4-Diaminobutyryl-piperidinamide.
PrSS11: HtrA1 inhibitor (Novartis).
HtrA2: Ucf-101 (Calbiochem).
Calpain 7: 3-(4-Iodophenyl)-2-mercapto-(Z)-2-propenoic Acid (Sigma-Eldrich).
Kallikrein 12: HAI-2A (R&D Systems), Ecotin (Sigma).
Cathepsin A: Ebelactone B: Chymostatin.
Cathepsin H: Human stefin A and human stefin B.
calpain-11: Calpastatin.

### EXAMPLE 15

### Inhibitory Cocktail for Bacterially Infected CSF Samples

There have been no reports of co-infection of viral and bacterial meningitis. Thus, a test for viral meningitis infection implies a negative result for bacterial meningitis. However, CSF samples infected with bacterial meningitis may have background non-specific activity that leads to false positive results in tests for enterovirus, thereby confusing the results. Background non specific activity in CSF samples could be related to bacterial proteases that can cleave the substrate, leading to a positive signal. The most frequent bacterial meningitis found in CSF include: Streptococcus pneumonia, Neisseria meningitides (meningococc), Haemophilus influenza and Klebsiella pneumonia. Accordingly, Pneumococcus (Streptococcus pneumonia, two different strains 6B and 23F) and meningococcus (Neisseria meningitides) were spiked to simulate the background non specific activity observed in CSF samples and to identify inhibitors to reduce it. A total of more than 40 inhibitors from different groups of inhibitors were tested.

Two inhibitors, Phosphoramidon and 2,6-pyridinedicarboxylic acid, significantly reduced the signal induced in these bacteria (FIGS. 22A-B). Phosphoramidon is a strong inhibitor of many bacterial Metalloendoproteinases, thermolysin, and elastase, but a weak inhibitor of collagenase. It does not inhibit trypsin, papain, chymotrypsin, and pepsin. Phosphoramidon was tested using the Echovirus 3C recombinant and pneumococcus (types 6B and 23F) spiking systems.

The experiment shown in FIG. 22A was performed in the presence of Echo recombinant protease (50 nM) or in the presence of two different strains of pneumococcus 6B and 23F (lysate 1 μl). The inhibitory effect was measured in the presence/absent of Phosphoramidon (20 uM). Phosphoramidon inhibited the recombinant protease by 7% and the 6B and 23F pneumococcus activity by 91% and 80% respectively. The results are representative of at least three independent experiments in which similar results of 90% inhibition on both strains was measured.

2,6-pyridinedicarboxylic acid was tested using the the Echovirus 3C recombinant and meningococcus spiking systems. The experiment shown in FIG. 22B was performed in the presence of Echo recombinant protease (1:18), in the presence of meningococcus (lysate 3 ul) or in the presence of blood (10 ul). The RFU/min value was determined in the presence/absence of 2,6-pyridinedicarboxylic acid (2.5 mM). 2,6-pyridinedicarboxylic acid inhibited the recombinant protease by 4.5%, the meningococcus activity by 81%, and blood activity by 0%.

Phosphoramidon and 2,6-pyridinedicarboxylic acid were therefore added to the inhibitor cocktail I8 describe above, and the inhibitors cocktails I9a (I8+Phosphoramidon), 19b (I8+2,6-pyridinedicarboxylic acid) and I10 (I8+Phosphoramidon+2,6-pyridinedicarboxylic acid) were established.

The final composition of I9a inhibitory cocktail is summarized in Table 14a.

TABLE 14a

| | | | final concentration |
|---|---|---|---|
| Name | Type | known target proteases for inhibition | used (uM) |
| AEBSF | serine protease | trypsin, chymotrypsin, plasmin, | 1000 |
| | inhibitor. | kallikrein, and thrombin | |
| Aprotinin | competitive serine | Acrosin, Chymotrypsin, | 7.5 |
| | protease | Chymotrypsinogen, Elastase, | |
| | | Kallikrein, Plasmin, Plasminogen | |
| Bestatin | synthetic dipeptide | leucine aminopeptidase, | 20 |
| | | aminopeptidase B, and triamino | |
| | | peptidase | |
| E-64 | irreversible cysteine | Actinidin, Ananain, Bromelain, | 50 |
| | protease | Calpain, Cathepsin B, Cathepsin B1, | |
| | | Cathepsin H, Cathepsin L, Cathepsin, | |
| | | Clostripain, Comosain, CMP-Sialic | |
| | | Acid: Lactosylceramide, α(2-3)- | |
| | | Sialytransferase, Ficin, α-Ginivain, | |
| | | Papain, α-and β-Trypsin | |
| Eglin C | 70 amino acid peptide | chymotrypsin, subtilisin, leukocyte, | 0.5 |
| | | elastase and cathepsin G. | |
| GW | | human neutrophil elastase | 10 |
| Pepstatin A | aspartyl proteases | pepsin, renin, cathepsin D, chymosin, | 10 |
| | | prorease B | |
| Heparin | mucopolysaccharide or | human elastase | 2.00 |
| | a glycosoaminoglycan. | | |
| Phosphoramidon | bacterial | thermolysin, and elastase | 91 |
| | metalloendoproteinases | | |

The final composition of I9b inhibitory cocktail is summarized in Table 14b.

TABLE 14b

| | | | final concentration |
|---|---|---|---|
| Name | Type | known target proteases for inhibition | used (uM) |
| AEBSF | serine protease | trypsin, chymotrypsin, plasmin, | 1000 |
| | inhibitor. | kallikrein, and thrombin | |
| Aprotinin | competitive serine | Acrosin, Chymotrypsin, | 7.5 |
| | protease | Chymotrypsinogen, Elastase, | |
| | | Kallikrein, Plasmin, Plasminogen | |
| Bestatin | synthetic dipeptide | leucine aminopeptidase, | 20 |
| | | aminopeptidase B, and triamino | |
| | | peptidase | |
| E-64 | irreversible cysteine | Actinidin, Ananain, Bromelain, | 50 |
| | protease | Calpain, Cathepsin B, Cathepsin B1, | |
| | | Cathepsin H, Cathepsin L, Cathepsin, | |
| | | Clostripain, Comosain, CMP-Sialic | |
| | | Acid: Lactosylceramide, α(2-3)- | |
| | | Sialytransferase, Ficin, α-Ginivain, | |
| | | Papain, α-and β-Trypsin | |
| Eglin C | 70 amino acid peptide | chymotrypsin, subtilisin, leukocyte, | 0.5 |
| | | elastase and cathepsin G. | |
| GW | | human neutrophil elastase | 10 |
| Pepstatin A | aspartyl proteases | pepsin, renin, cathepsin D, chymosin, | 10 |
| | | prorease B | |
| Heparin | mucopolysaccharide or | human elastase | 2.00 |
| | a glycosoaminoglycan. | | |
| 2,6 | Broad spectrum | | 2500 |
| pyridinedicarboxylic | inhibitor of | | |
| acid | metalloproteases | | |

The final composition of I10 inhibitory cocktail is summarized in Table 15.

TABLE 15

| | | | | Volume from stock | Concentration |
|---|---|---|---|---|---|
| | | Stock | Preparation | to inhibitors | (μM) |
| Material Name | Stock Solvent | Strength | Of stock | mixture tube (μl) | In the well |
| AEBSF | 25 mM HEPES, | 200X | 25 mg/0.52 ml | 5 ul | 333 |
| | pH 5 | | | | |
| Aprotinin | 25 mM HEPES, | 200X | 5 mg/0.5 ml | 15 ul | 7.5 |
| | pH 7.5 | | | | |
| Bestatin | DMSO | 200X | 0.8 mg/0.6 ml | 15 ul | 20 |
| EglinC | 25 mM HEPES, | 200X | 0.1 mg/0.25 ml | 15 ul | 0.5 |
| | pH 7.5 | | | | |
| GW | 25 mM HEPES, | 200X | 1 mg/1.15 ml | 15 ul | 10 |
| | pH 7.5 | | | | |
| PepstatinA | DMSO | 200X | 0.82 mg/0.6 ml | 15 ul | 10 |
| Heparin | 25 mM HEPES, | 200X | 24 mg/1 ml | 15 ul | 2 |
| | pH 7.5 | | | | |
| Phosphoramidon | 25 mM HEPES, | 200X | 5 mg/1 ml | 15 ul | 91 |
| | pH 7.5 | | | | |
| 2,6 | DMSO | 100X | 42 mg/1 ml | 30 ul | 2500 |
| pyridinedicarboxylic | | | | | |
| acid | | | | | |
| E-64 | 25 mM HEPES, | 200X | 5 mg/1.4 ml | 15 ul | 50 |
| | pH 7.5 | | | | |

Enterovirus 3C protease (Echo), WBC lysate (corresponding to 2×103 cells per test), Pneomococc lysate (corresponding to 1×10⁶ bacteria per test) and Meningococc lysate (corresponding to 1×10⁸ bacteria per test) (5 ul each) were spiked into artificial-CSF (45 ul), followed by the addition of the indicated inhibitor cocktails (5 ul each): I8, I9a=I8+Phosphoramidon (90 μM), I9b=I8+2-6-pyridinedicarboxylic acid (2.5 mM) and I10=I8+Phosphoramidon+2-6-pyridinedicarboxylic acid. The samples with the indicated inhibitor cocktails were incubated at RT for 2.5 min followed by the addition of 50 ul 2× reaction buffer containing the substrate camb2 (final concentration 2 μM). Fluorimetric measurements were started immediately. WBC lysate simulate WBC death and tissue inflammation effect, which causes the release of protease from cells undergoing apoptosis in the samples. Bacterial amounts are correlated to mean (Pneomococc) and high (Meningococc) concentration found in 50 μl of infected CSF samples.

The results in FIG. 23 show that the I10, I9a and I9b inhibitor cocktails inhibits the Enterovirus 3C protease by ˜20% and the WBC lysate by ˜90%. These results indicate that these inhibitors cocktails can block CSF proteolytic activity related to WBC death, without interfering with protease activity related to the Enterovirus 3C protease. On the other hand, while the I10 mixture inhibits proteolytic activity of both bacteria lysates by 80%-90%, the I9a and I9b inhibitors cocktails had specific inhibition activity towards select bacteria. The I9a inhibits the Pneomococc proteolytic activity, without affecting the Meningococc proteolytic activity, and I9b inhibits the Meningococc proteolytic activity but not the one induced by Pneomococc.

A theoretical example of a meningitis test result is shown in Table 16. The test is performed in 5 tubes. The first 3 tubes are incubated with the I10 inhibitor cocktail: negative control (artificial CSF), positive control (artificial CSF spiked with recombinant Enterovirus 3C protease) and a sample of CSF. Two more tubes containing the CSF sample will be incubated with the inhibitor cocktail I9a or I9b.

If no signal is detected, the sample can be considered as negative for both Enterovirus and bacteria (or under detection limits of the assay) (Result #1 in Table 16). As no co-infection of virus and bacteria has been reported, a positive signal in the presence of I10 indicates the presence of live Enterovirus in the tested CSF sample (Result #2). As I9b contains 2-6-pyridinedicarboxylic acid, it will inhibit only the Meningococcus activity. Therefore, a positive signal in tube #4 (incubated with I9a) and a negative signal in tubes #3 and 5 (incubated with I10 and I9b that contains 2-6-pyridinedicarboxylic acid) will indicate the presence of Meningococcus in the sample (Result #3). As I9a contains Phosphoramidon, it will inhibit only the Pneumococcus activity. Therefore, a positive signal in tube #5 (incubated with I9b) and a negative signal in tubes #3 and 4 (incubated with I10 and I9b that contains Phosphoramidon) will indicate the presence of Pneumococcus in the sample (Result #4).

Accordingly, the methods and compositions disclosed in WO 2007/029262 for detecting a viral meningitis infection can be used in conjunction with the inhibitors of the present methods. For example, the substrates that can be used for detecting a viral meningitis infection include those that detect herpes virus, West Nile virus, and enterovirus. A specific example of a substrate that can be used for the detection of herpes virus includes SEQ ID NO: 48. Examples of substrates that can be used for the detection of West Nile virus include SEQ ID NOs: 49, 50, and 51. Examples of substrates that can be used for the detection of enterovirus include SEQ ID NOs: 52, 53, 54, 55, 56, 57, and 58. In addition, the inhibitors may be used to distinguish between bacterial and viral meningitis infection.

TABLE 16
Test and results interpretation

| | | | Result | Result | Result | Result |
|---|---|---|---|---|---|---|
| Tube # | Sample | Inhibitors | #1 | #2 | #3 | #4 |
| 1 | Positive | I10 | + | + | + | + |
| | control | | | | | |
| 2 | Negative | I10 | − | − | − | − |
| | control | | | | | |
| 3 | CSF | I10 | − | + | − | − |
| 4 | CSF | I9a | − | + | + | − |
| 5 | CSF | I9b | − | + | − | + |
| Menin- | | | Non- | Echo | Meningo | Pneomo |
| gitis | | | Echo | | | |

### EXAMPLE 16

### Analysis of Clinical Samples for Enterovirus Before Assay Optimization

CSF samples obtained from two clinical sites, were tested for presence of enterovirus according to the method of the invention but before the assay conditions were optimized. The samples were also tested with a combination of RT-PCR and other clinical parameters. In the RT-PCR/clinical parameter combination, a sample was considered positive only when it exhibited inflammation, i.e. above 7 white blood cells (WBC) in 1 uL CSF and RT-PCR is positive. A sample was considered negative when it exhibited no inflammation (<=7 cell/ul) or when it exhibited inflammation but was negative with RT-PCR. Samples were considered hemolytic if red blood cell (RBC) count was above 100/ul (for the one set) or if the sample's color was reddish (for the other set, RBC count not provided).

Reaction rate cutoff value was first determined for the method of the invention retrospectively so that it exhibited the best correlation between specificity and sensitivity. Samples whose reaction rate exceeded the cutoff value were considered positive, and those below this value were considered negative.

### Procedure for Detecting Enterovirus According to the Method of the Invention

Each measurement was performed by using 50 uL of CSF sample. 5 ul of inhibitor cocktail (I8) was added to the samples and incubated for 2.5 min. The reaction was initiated by the addition of 50 uL 2× reaction buffer (“2× RB”, 50 mM HEPES pH7.5, 1.8M Na₂SO4, 300 mM NaCl, 10% Glycerol, 10 mM EDTA and 10 mM EGTA each) containing the substrate camb2 (final concentration 2 uM).

### Clinical Site 1: Prospective Samples f

46 leftover CSF samples from one clinical site were collected from prospective patients as approved by the Helsinki committee. Samples were sent to a virology laboratory for RT-PCR analysis. The method of the invention was performed as described above.

Table 17 summarizes the results from the clinical site. The reaction rate cutoff that represents the best correlation was set at 335 RFU/min (about 20% of positive control; 50 nM recombinant 3C protease).

TABLE 17
CSF samples from One Clinical Site.

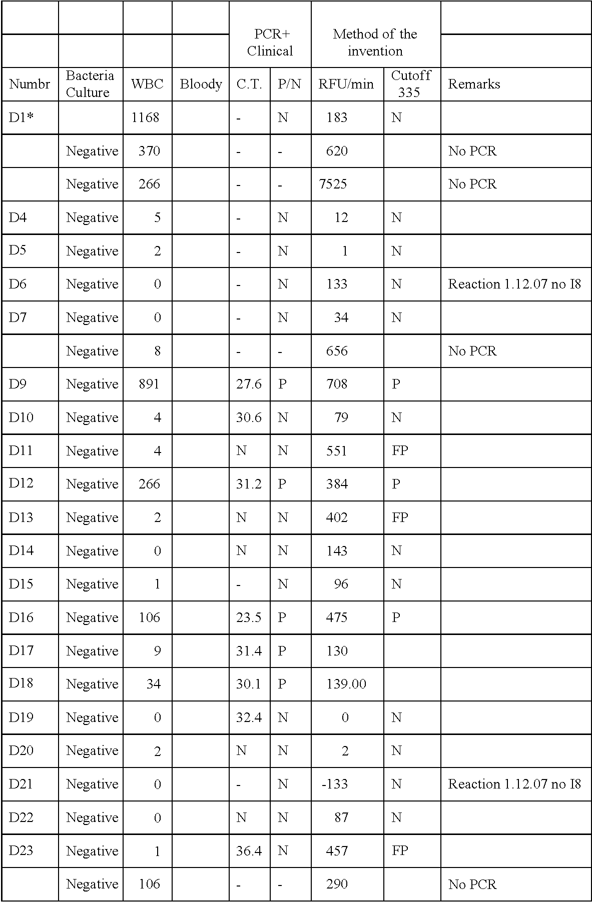

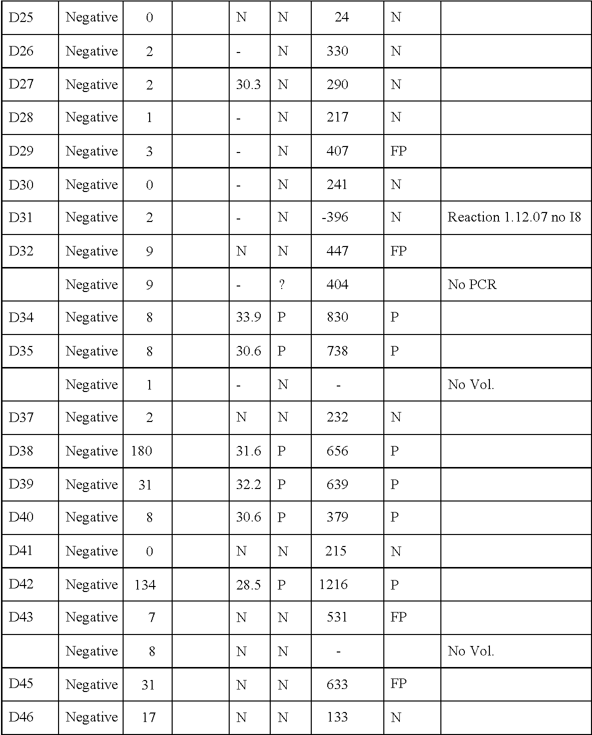

Legend:
B—Hemolytic,
P—positive,
N—negative,
FP—False Positive,
FN—False Negative,
X—excluded due to insufficient data.
*The need for inhibitors cocktail: Initially the experiment with the set of samples was performed without protease inhibitors cocktail. However, sample D1 had tested positive for streptococcus pneumococcus and could not be distinguished from positive viral samples. Therefore, the experiment was repeated with the addition cocktail I8 for 2.5 min prior to the addition of the reaction buffer. As a result, sample D1 tested negative, while other viral positive samples remained positive. Overall, a better correlation to the RT-PCR method could be obtained by adding the inhibitor cocktail I8. The cutoff was reduced by 45% from 600 (without inhibitors) to 335 RFU/min (with inhibitors). Interestingly, a similar reduction of 40% was observed when recombinant protease activity was measured with and without inhibitors. This result reinforces the observation that the activity in positive CSF samples is due to 3C protease activity.

### Clinical Site 2: Retrospective Samples

30 positive and 30 negative CSF samples pre-tested for Enterovirus were collected retrospectively leftover from another viral laboratory under the approval of the Helsinki committee. The method was compared to RT-PCR only because the samples had already passed the clinical criteria for inflammation (>7 WBC/ul) before being sent for RT-PCR testing. The method of the invention was performed as described above.

Table 18 summarizes the results of these samples. The same cutoff of 335 RFU/min (about 20% of positive control; 50 nM recombinant 3C protease) was used.

TABLE 18
CSF samples from Clinical Site 2.

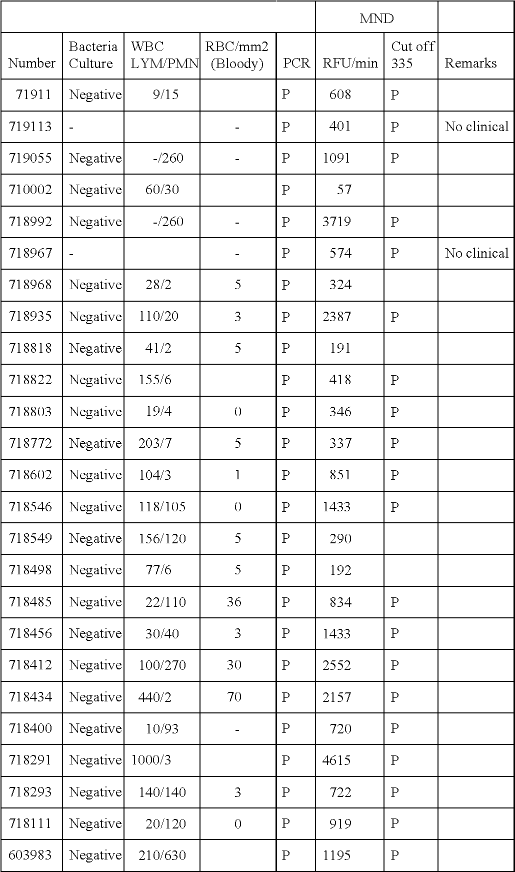

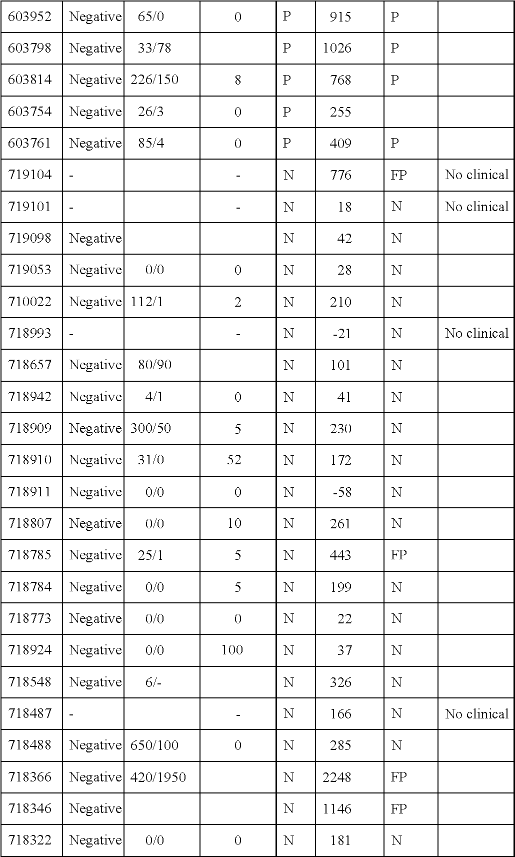

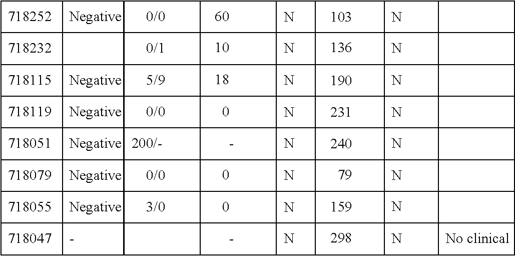

Legend:
B—Hemolytic,
P—positive,
N—negative,
FP—False Positive,
FN—False Negative.

### Clinical Results Analysis

### Hemolytic Samples

The results demonstrate that the assay may not deal efficiently with samples containing red blood cells (above 100 RBC or samples appearing red). The assay failed to determine correctly 5 out of 17 hemolytic samples. Of the five, four were false positive and one false negative. Of the remaining 12, 9 were positive and 3 negative. Thus, red blood cells may be removed from the sample by, for example, centrifugation before analysis. Moreover, the red blood cells can be removed before freezing the samples for storage.

### False Negative

The assay failed to determine correctly 8 (710002, 718968, 718818, 718549, 718498, 603754, D17 and D18) out of 41 positive samples (one of which was bloody). To rule out the possibility that the false negative samples inhibited the protease, all samples except D17 were spiked with 50 nM recombinant enzyme. No inhibition was detected in these 7 samples. These false negative could be due to lower sensitivity of the instant method compared to RT-PCR. Alternatively, the protease may have been inactivated due to storage or freeze/thaw cycles that the samples had undergone.

### False Positive

11 out of 58 samples were false positive (D11, D13, D23, D29, D32, D43, D45, 719104, 718785, 718366, and 718346), 4 of which were bloody (D32, D45, 718366, and 718346).

False positive (FP) signals may have originated from unspecific substrate cleavage due to inflammation factors. However, there was no correlation between the WBC count and the magnitude of unspecific background noise. Moreover, 9 negative samples (D1, D46, 710022, 718657, 718909, 718910, 718488, 718115, and 718051) with inflammation gave a signal below the cutoff value, suggesting that instant method can accommodate inflammatory samples.

5 of the FP samples were without inflammation (D11, D13, D23, D29 and D43). This suggests that if hemolytic and FP samples with no inflammation are not included, only two FP samples remain.

### RT-PCR to Clinical Parameters Contradictions

4 samples showed negative clinical characteristics (no inflammation) and tested RT-PCR positive (D10, D19, D23 and D27). Of these, only D23 gave a positive signal in the instant method. This could result from any of the following reasons: false positive of the RT-PCR signal or viral genomic material leftovers with no active infection.

### Conclusions

Over 100 CSF samples were used to evaluate a method of detecting enterovirus meningitis according to the instant invention. Table 19 summarizes the results. Sensitivity and specificity using the method of the invention were 80 and 81%, respectively. Higher specificity (86%) can be obtained if hemolytic samples are excluded.

TABLE 19

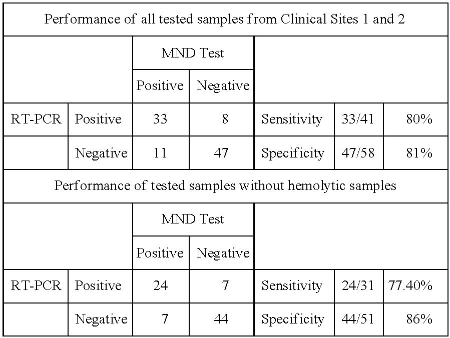

### EXAMPLE 17

### Assay Optimization—Plates and Tubes

### Reaction Plastics

### Background:

A negative slope in blank reactions was observed. Among other reasons, it was concluded that part of this could be due to adherence of substrate to the plate wells during the reaction. Another observation was that after centrifugation, the substrate pellet would stick all around the sides of the tube inner wall. Thus, nonbinding (NB) plates and tubes were tested.

### Results:

Plates

Black plates from Greiner cat. 655900 were obtained and tested. To test for substrate adherence, a standard assay buffer (+substrate) was incubated in both regular and NB plates for 15 min. Each well was washed twice with 200 ul PBS and vortexed vigorously. Finally, to evaluate how much is left stuck to the plate, wells were re-suspended in 100 ul 1× reaction buffer. Each wash was measured for fluorescence. Results are shown in Table 20.

TABLE 20

| | Regular plate, RFU | Non binding plate, RFU |
|---|---|---|
| Wash 1 | 8500 | 10000 |
| Wash 2 | 4000 | 1500 |
| Left stuck | 2100 | 730 |

The results indicate that in the first wash, more substrate was washed out in NB plates. After the second wash, regular plates retained ˜3 fold more substrate than NB. Finally, after 2 washes, regular plates retained ˜3 fold more substrate than NB. An additional feature of NB plates is its effect on the blank curve shape. The wave shaped blank curve typical to regular (FIG. 24A) plates became more linear with a slight positive slope (FIG. 24B, ˜200 RFU/min).

Another feature of the NB plate is its contribution to reduce c.v. values. As seen in Table 21, typical c.v. values with NB plates are 5-6% compared to 10-15% in regular plates.

TABLE 21
REPRODUCIBILITY WITH NON-BINDING MICROPLATE

| | BL | P.C |
|---|---|---|
| EXP-1 | 192 | 4280.2 |
| | 272 | 4005.3 |
| | 367 | 4407.8 |
| | 347 | 4610.4 |
| | | 4136.3 |
| | | 3809.8 |
| | | 3787.2 |
| | | 4132 |
| Average | 294.5 | 4146.125 |
| stdev | 79.63458 | 283.8688 |
| c.v. | 27.04061 | 6.846606 |
| EXP-2 | 200 | 4136.6 |
| | 248 | 4797.3 |
| | 154 | 4335.4 |
| | 217 | 4489.8 |
| | | 4292.2 |
| | | 4515.1 |
| | | 4761 |
| Average | 204.75 | 4475.343 |
| stdev | 39.23752 | 243.2139 |
| c.v. | 19.16363 | 5.434532 |

### Tubes

### Background:

It was noticed that that reaction rate (RFU±min) for blank and positive control reactions varies significantly when using amber vs. NB tubes. Blank values (RFU±min) and baseline fluorescence were higher in NB tubes. Thus, the effect of amber vs. NB tubes on baseline fluorescence, blank and positive control reaction rates were examined.

### Reagents and Instruments:

Inhibitor Cocktail: Inhibitors cocktail I10
Enzyme dilution buffer: −25 mM HEPES pH 7.5, 150 mM NaCl, 1 mM EDTA, 10% Glycerol, 0.5 mg/ml BSA, 1M Sucrose, 0.1% Sodium azid.
2× reaction buffer, Buffer B: 50 mM HEPES, 10 mM EDTA, 10 mM EGTA, 1.2M Na2SO4, sodium azide 0.1%, pH 8.5
Artificial CSF: NaCl 125 mM, KCl 2.5 mM, MgCl 1 mM, NaH₂PO₄ 1.25 mM, CaCl₂ 2 mM, Glucose 2.5 mM, NaHCO₃ 25 mM, 0.1% Sodium azide, HEPES 25 mM, pH 7.45.
Enzyme: Recombinant protease from enterovirus strain “Echo” [dilution 1:18
Substrate: Camb-2.3, 1 mM, Camb-2.4, 2 mM. (Different batches for the same Camb2 sequence)
HEPES Buffer: HEPES 25 mM
NB tubes: Sigma Cat #Z666505
Amber tubes: USA scientific Inc. Cat. 1620-2707
96 well Plate: Dark bottom NB plate—Greiner Cat. 655900

| Fluorimeter | Fluorimeter parameters |
|---|---|
| DMV, Polar-Star Galaxy | Ex. 520-10 mm, Em. 570-20 mm. |
| | Temprature RT. |
| | Total reading Time X min. |
| | Read each 4 sec. |
| | Gain 90 (RB) gain 80 (HEPES) |

### Evaluation of the Difference in Parameters of the Tubes

### Preparations:

Substrate: camb 2.3 and 2,4 were diluted to 0.333 mM with DMSO.
Further dilution of 1:83 in HEPES buffer±2× RB was performed. (4.8 ul substrate for 400 ul RB±Hepes for a working concentration).

Well contents were as according to Table 22

TABLE 22

| | Negative | Positive |
|---|---|---|
| Test tube ± Item | Control | control |
| artiCSF ± HEPES buffer | 50 | 45 |
| Echo 1:18 | — | 5 |
| HEPES buffer ± RB + | 50 | 50 |
| substrate | | |
| Total volume | 100 | 100 |

1. Add artiCSF±HEPES buffer to plate
2. Prepare 400 ul HEPES buffer±RB+substrate (camb2.3 and 2.4) in both amber and NB tubes.
3. Add enzyme to wells
4. Add HEPES buffer±RB+substrate to wells with multichannel pipette.

### Results

Results are shown in Tables 23, 24, and 25 and FIGS. 25A and 25B.

From five independent repeats, it is observed that baseline fluorescence and blank rate are 50% higher in Lo binding than in amber tubes for both camb2.3 and 2.4. Nonetheless, reaction rates (minus blank) remained relatively the same. A 25% increase in average reaction rate was observed only in camb2.4 NB tubes. To verify if these differences are salt dependent, the same experiment was repeated 3 independent times in HEPES buffer. Difference in baseline fluorescence was reduced to 30-40% in amber vs. NB tubes, respectively, suggesting that at least part, this phenomenon in due to a salt effect.

Different fluorescence characteristics and reaction rates were observed in the amber vs. NB tubes. Thus, the cause of the difference was further investigated.

TABLE 23
Total fluorescence and activity readings following substrate preparation in Amber
vials using 2xRB for dilution

| Camb 2.4 substrate | | | | Camb 2.3 substrate | | | |
|---|---|---|---|---|---|---|---|
| Total | | | | Total | | | |
| RFU at | | | Positive | RFU at | | | Positive |
| T0 | | Positive | control- | T0 | | Positive | control- |
| (kRFU) | Blank | control | blank | (kRFU) | Blank | control | blank |
| 11.8 ± 0.1 | 287 ± 55 | 1376 ± 40 | 1089 | 26.4 ± 0.14 | 453 ± 41 | 1812 ± 79 | 1359 |
| 14.8 ± 0.14 | 143 ± 31 | 1782 ± 97 | 1639 | 32.1 ± 0.13 | 527 ± 41 | 2246 ± 97 | 1719 |
| 13.6 ± 0.23 | 147 ± 33 | 1526 ± 220 | 1379 | 30.4 ± 0.6 | 485 ± 56 | 1958 ± 66 | 1473 |
| 15.3 ± 0.09 | 199 ± 42 | 1289 ± 82 | 1090 | 31.9 ± 0.4 | 403 ± 48 | 1557 ± 59 | 1154 |
| 13.1 ± 0.1 | 237 ± 22 | 1182 ± 60 | 945 | 29.9 ± 0.3 | 344 ± 53 | 1444 ± 26 | 1100 |
| | | Average | 1228.4 | | | Average | 1361 |
| | | SD | 278.4669 | | | SD | 250.8396 |

TABLE 24
Total fluorescence and activity readings following substrate preparation in Lo binding
vials using 2xRB for dilution

| Camb 2.4 substrate | | | | Camb 2.3 substrate | | | |
|---|---|---|---|---|---|---|---|
| Total | | | | Total | | | |
| RFU at | | | Positive | RFU at | | | Positive |
| T0 | | Positive | control - | T0 | | Positive | control- |
| (kRFU) | Blank | control | blank | (kRFU) | Blank | control | blank |
| 23.2 ± 0.9 | 732 ± 104 | 2398 ± 50 | 1666 | 41.9 ± 0.2 | 814 ± 72 | 2209 ± 21 | 1395 |
| 25.6 ± 0.19 | 648 ± 107 | 2647 ± 93 | 1999 | 45.5 ± 0.7 | 1053 ± 73 | 2654 ± 141 | 1601 |
| 23.8 ± 0.3 | 548 ± 62 | 2089 ± 101 | 1541 | 43.6 ± 0.19 | 1036 ± 27 | 2373 ± 110 | 1337 |
| 24.5 ± 0.2 | 550 ± 44 | 2173 ± 63 | 1623 | 40.5 ± 0.4 | 977 ± 11 | 1989 ± 37 | 1012 |
| 26.4 ± 0.3 | 463 ± 93 | 1770 ± 202 | 1307 | 42.4 ± 0.3 | 840 ± 65 | 1817 ± 107 | 977 |
| | | Average | 1627.2 | | | Average | 1264.4 |
| | | SD | 249.8584 | | | SD | 265.486 |

TABLE 25
Total fluorescence (kRFU) following substrate
(Camb 2.3 and Camb2.4) preparation in Amber or
Lo binding vials using Hepes for dilution

| | |
|---|---|
| Amber | Lo binding |

| Camb 2.4 | Camb 2.3 | Camb 2.4 | Camb 2.3 |
|---|---|---|---|
| 20.3 ± 1 | 35.5 ± 0.077 | 28.7 ± 0.1 | 41.3 ± 0.7 |
| 22.6 ± 0.3 | 37.7 ± 0.5 | 29 ± 0.7 | 43.4 ± 0.3 |
| 21.5 ± 0.3 | 34 ± 0.1 | 34 ± 0.2 | 44 ± 0.1 |

### Evaluation of Whether Absorption or Release of Fluorescence is the Cause of the Differences in Fluorescence and Reaction Rate

Substrate: camb 2,4 was diluted to 0.333 mM with DMSO.
Further dilution of 1:83 in 2× RB was performed. (4.8 ul substrate for 400 ul RB for a working concentration)

Well content was as according to Table 26.

TABLE 26

| | Negative |
|---|---|
| Test tube ± Item | Control |
| artiCSF | 50 |
| Echo 1:18 | — |
| RB + substrate | 50 |
| Total volume | 100 |

1. Add artiCSr to plate
2. Prepare 3×400 ul RB+substrate in both amber and NB tubes. Incubate at RT 5 min.
3. Transfer to fresh amber/NB tube. Incubate at RT 5 min.
4. Add RB+substrate to wells.

Results

Results are shown in Table 27.

The reason for the different properties appears to be absorption to the amber tubes. RB+substrate that were passed through amber tubes shows the lower RFU reading. When passed twice in amber tubes, the lowest readings were obtained. When RB was passed twice through NB tubes, no significant change in RFU was observed.

Furthermore, it was observed that RFU/min correlated to the RFU start point (R2=0.941). This may suggest that the main influence on blank rate is due to substrate concentration.

TABLE 27

| | RFU | Rate |
|---|---|---|
| | start | RFU/min |
| One amb only | 15455 | 234 |
| amb to NB | 17702 | 423 |
| amb to amb | 8790 | 54 |
| One NB only | 35313 | 888 |
| NB to amb | 24700 | 807 |
| NB to NB | 38473 | 1153 |

### Evaluation of Whether Active or Non Active Material is Absorbed to the Tubes

Substrate: camb 2,4 was diluted to 0.333 mM with DMSO.
Further dilution of 1:83 in 2× RB was performed (2 uM final, 4.8 ul substrate for 400 ul RB) in both amber an NB tubes.
Further dilution of 1:46 in 2× RB was performed (4 uM final, 9.6 ul substrate for 400 ul RB) in amber tubes.
Further dilution of 1:166 in 2× RB was performed (1 uM final, 2.4 ul substrate for 400 ul RB) in NB tubes.

Well contents were as according to Table 28.

TABLE 28

| | Negative | Positive |
|---|---|---|
| Test tube ± Item | Control | control |
| artiCSF | 45 | 45 |
| Dilution buffer | 5 | — |
| Echo 1:18 | — | 5 |
| RB + substrate | 50 | 50 |
| Total volume | 100 | 100 |

1. Add artiCSF±HEPES buffer to plate
2. Prepare 400 ul RB+substrate in both amber and NB tubes.
3. Add enzyme/dilution buffer to wells.
4. Add RB+substrate to wells.

### Results

The results are shown in Tables 29 and 30. It appears that at least part of the material absorbed is active material. Substrate concentration was adjusted such that baseline fluorescence is about the same in both NB and amber tubes (4 uM and 2 uM in amber and NB tubes, respectively or 2 uM and 1 uM in amber and NB tubes, respectively). The blank and reaction rates were close in both tubes.

TABLE 29

| amber | | | | | | | |
|---|---|---|---|---|---|---|---|
| 2 uM | | | | 4 uM | | | |
| camb | | | | camb | | | |
| RFU | | | | RFU | | | |
| start | B | pc | pc-B | start | B | pc | pc-B |
| 18.3 | 390 | 1587 | 1197 | 31.7 | 1059 | 2948 | 1889 |
| 16.6 | 440 | 2003 | 1563 | 36.4 | 1110 | 3144 | 2034 |

TABLE 30

| NB | | | | | | | |
|---|---|---|---|---|---|---|---|
| 2 uM | | | | 1 uM | | | |
| camb | | | | camb | | | |
| RFU | | | | RFU | | | |
| start | B | pc | pc-B | start | B | pc | pc-B |
| 31.7 | 713 | 2307 | 1594 | 16.8 | 388 | 1536 | 1148 |
| 30.5 | 773 | 2847 | 2074 | 19.4 | 318 | 2024 | 1706 |

### EXAMPLE 18

### Assay Optimization—Buffer

The reaction buffer is a part of the enterovirus test kit of the invention. The camb-2 substrate is dissolved in the reaction buffer. The reaction buffer contains different salts to allow the tested enzyme to work at the optimal conditions. The current reaction buffer composition is: HEPES pH 7.5, 300 mM NaCl, 10 mM EDTA, 10 mM EGTA, 10% Glycerol, 1.8M Na2SO4, 0.1% Sodium azide. The amount of salt in the reaction buffer is high and allows optimal enzymatic activity. However, under these conditions, substrate solubility is affected. Thus, the reaction buffer was improved to increase substrate solubility without reducing enzymatic activity of the tested 3C protease.

### Results:

300 mM NaCl and 10% glycerol was removed from the reaction buffer and several reduced concentrations of Na2SO4 (starting at 0.9M) were tested in order to determine the minimal salt requirements of the assay (Tables 31-34). Experiments were repeated at least three times by two different scientists.

TABLE 31
Determining the need for NaCl and Glycerol in the reaction buffer at 0.9M Na2SO4
(n = 9)

| | | | | | | | 0.9M Na₂SO₄ | |
|---|---|---|---|---|---|---|---|---|
| | Original buffer | | 0.9M Na₂SO₄ | | 0.9M Na₂SO₄ | | No NaCl, | |
| | 0.9M Na₂SO₄ | | No NaCl | | No Glycerol | | No Glycerol | |
| | blank | * p.c. | blank | p.c | blank | p.c | blank | p.c. |
| EXP-1 | −938.84 | 1636.76 ± 4.7 | −731.25 | 2005.69 ± 3.3 | −685.33 | 1834.58 ± 2.67 | −1070.96 | 2081.3 ± 7.1 |
| EXP-2 | −1047.9 | 1791.82 ± 3.3 | −619.38 | 2367.11 ± 6.9 | −904.31 | 1829.44 ± 4.89 | −716.56 | 2740.3 ± 5.3 |
| EXP-3 | −131.84 | 1239.87 ± 9.7 | −565.49 | 1497.55 ± 6.2 | −855.01 | 1233.36 ± 7.9 | −911.25 | 1314.6 ± 5.7 |

* p.c. = positive control

TABLE 32
Determining the need of NaCl and Glycerol in the reaction buffer at 0.8M Na2SO4
(n = 3)

| | | | | | | | 0.8M Na₂SO₄ | |
|---|---|---|---|---|---|---|---|---|
| | 0.8M Na₂SO₄ | | 0.8M Na₂SO₄ | | 0.8M Na₂SO₄ | | No NaCl, | |
| | Original buffer | | No NaCl | | No Glycerol | | No Glycerol | |
| | blank | p.c. | blank | p.c | blank | p.c | blank | p.c. |
| EXP-1 | −972.70 | 2212.7 ± 15.7 | −541.65 | 2798.13 ± 0.6 | −638.06 | 2208.13 ± 2.31 | −923.25 | 2865.2 ± 8.2 |
| EXP-2 | −659.26 | 1468.4 ± 16.6 | −172.69 | 1699.3 ± 14.2 | −587.94 | 2033.72 ± 11.0 | −432.26 | 2314.2 ± 5.2 |
| EXP-3 | −512.21 | 1677.9 ± 15.9 | −95.35 | 2351.6 ± 13.8 | −721.09 | 2471.16 ± 10.3 | −625.97 | 2478.6 ± 3.4 |
| EXP-4 | −130.33 | 998 ± 18.3 | −1383.6 | 1248.6 ± 11.3 | −117 | 875.33 ± 15.1 | −203 | 1444 ± 3.75 |

TABLE 33
Determining the need of NaCl and Glycerol in the reaction buffer at 0.7M Na₂SO₄
(n= 3)

| | | | | | | | 0.7M Na₂SO₄ | |
|---|---|---|---|---|---|---|---|---|
| | 0.7M Na₂SO₄ | | 0.7M Na₂SO₄ | | 0.7M Na₂SO₄ | | No NaC1, | |
| | Original buffer | | No NaCl | | No Glycerol | | No Glycerol | |
| | blank | p.c. | blank | p.c | blank | p.c | blank | p.c. |
| EXP-1 | −191.01 | 1492.7 ± 27.0 | −103.33 | 1454.7 ± 45.5 | −307.76 | 1717.5 ± 23.9 | −560.3 | 2336.4 ± 9.8 |
| EXP-2 | −504.97 | 2046.1 ± 5.98 | 52.8 | 2533.8 ± 8.19 | −783.2 | 1481.05 ± 122 | −812.93 | 2739.9 ± 22 |
| EXP-3 | 44.34 | 2016.3 ± 11.2 | 284.6 | 1949.9 ± 6.37 | −455.75 | 1526.5 ± 8.36 | −245.05 | 2539.7 ± 5.1 |
| EXP-4 | −401.5 | 1263 ± 10.06 | — | — | −187.6 | 1227 ± 215.6 | −101 | 1534.6 ± 9.8 |

TABLE 34
Determining the need for NaCl and Glycerol in the reaction buffer at 0.6M Na₂SO₄
(n = 3)

| | | | | | | | 0.6M Na₂SO₄ | |
|---|---|---|---|---|---|---|---|---|
| | 0.6M Na₂SO₄ | | 0.6M Na₂SO₄ | | 0.6M Na₂SO₄ | | No NaCl, | |
| | Original buffer | | No NaCl | | No Glycerol | | No Glycerol | |
| | blank | p.c. | blank | p.c | blank | p.c | blank | p.c. |
| EXP-1 | −90.52 | 1308.6 ± 1.35 | −67.33 | 1454.7 ± 45.5 | 35.68 | 1276.1 ± 6.64 | −560.3 | 2336.4 ± 9.8 |
| EXP-2 | −44.72 | 814.28 ± 14.4 | 432.08 | 714.72 ± 71.4 | 466.66 | 1430.6 ± 5.04 | 266.38 | 1591.4 ± 3.4 |
| EXP-3 | 97.66 | 778.6 ± 21.4 | 347.3 | 1017.6 ± 63.7 | 18 | 1168 ± 13.28 | 160.29 | 2072.7 ± 6.1 |

The above results show that at each Na₂SO4 concentrations tested (0.9-0.6M—Tables 31-34), the enzymatic activity was improved when both 300 mM NaCl and 10% glycerol were removed. A slight improvement of the CVs was also observed. Therefore, the reaction buffer was modified by removing NaCl and Glycerol. Next, the Na₂SO4 concentration was tested for the minimally acceptable level without affecting enzymatic activity (Tables 35-36).

TABLE 35
Reduction of Na₂SO₄ concentration (n = 3)

| | 0.9M | | 0.8M | | 0.7M | | 0.6M | |
|---|---|---|---|---|---|---|---|---|
| | No NaCl, | | No NaCl, | | No NaCl, | | No NaCl, | |
| | No Glycerol | | No Glycerol | | No Glycerol | | No Glycerol | |
| | blank | p.c. | blank | p.c | blank | p.c | blank | p.c. |
| EXP-1 | −834.06 | 1688.1 ± 6.82 | −657.4 | 2296.2 ± 7.61 | −494.98 | 1677.24 ± 2.42 | 213.93 | 2073.3 ± 3.3 |
| EXP-2 | −680.18 | 1637.0 ± 2.27 | −901.12 | 1919.5 ± 3.13 | −719.28 | 1846.02 ± 14.4 | 503.39 | 2223.1 ± 6.76 |
| EXP-3 | −1168.8 | 2365.3 ± 9.6 | −1117.4 | 2637.66 ± 7.4 | −806.5 | 2809.97 ± 21.5 | 495.82 | 2452.3 ± 12.6 |

The results indicate that 0.6M with no NaCl and no Glycerol is equal or more efficient within the CV variation than at other Na₂SO4 concentrations tested. Thus, 0.6M was chosen. Lowering Na₂SO4 concentration below 0.6M required a lower gain setting, indicating a loss of assay sensitivity. Below 0.4M Na₂SO4, a sharp reduction in reaction rate was observed (Table 36).

TABLE 36
Reduction of Na2SO4 concentration with lowering gain (n = 3)

| | 0.9M | | 0.8M | | 0.7M | | 0.6M | |
|---|---|---|---|---|---|---|---|---|
| | No NaCl, | | No NaCl, | | No NaCl, | | No NaCl, | |
| | No Glycerol | | No Glycerol | | No Glycerol | | No Glycerol | |
| | blank | p.c. | blank | p.c | blank | p.c | blank | p.c. |
| EXP-1 | −834.06 | 1688.1 ± 6.82 | −657.4 | 2296.2 ± 7.61 | −494.98 | 1677.24 ± 2.42 | 213.93 | 2073.3 ± 3.3 |
| EXP-2 | −680.18 | 1637.0 ± 2.27 | −901.12 | 1919.5 ± 3.13 | −719.28 | 1846.02 ± 14.4 | 503.39 | 2223.1 ± 6.76 |
| EXP-3 | −1168.8 | 2365.3 ± 9.6 | −1117.4 | 2637.66 ± 7.4 | −806.5 | 2809.97 ± 21.5 | 495.82 | 2452.3 ± 12.6 |

Reproducibility Tests with 0.6M Na₂SO4

CSF sample was incubated with 50 nM Echo protease and 2× reaction buffer with 0.6M Na₂SO4. Also included were 3 blanks and 9 positive control repetitions. One representative experiment is shown in Table 37.

TABLE 37

| | RFU/min |
|---|---|
| | 50 nM Echo |
| | 3500.308 |
| | 3984.629 |
| | 3825.399 |
| | 3772.385 |
| | 4286.979 |
| | 4141.972 |
| | 3563.895 |
| | 4308.713 |
| | 4365.755 |
| Average | 3972.226 |
| STDEV | 306.786 |
| CV | 7.723 |

The reproducibility experiments of 2× reaction buffer with 0.6M Na₂SO4 exhibit the same or a better reaction rate as compared to a buffer containing 0.9M Na₂SO4.

The new buffer composition was then tested on two positive and two negative CSF samples. The results (Table 38) indicate that regardless of buffer composition, the samples tested maintained the same features. A slight increase in reaction rate with the new buffer was observed. Due to this rise, it may be necessary to increase the cutoff value in future tests.

TABLE 38

| Sample's | | | |
|---|---|---|---|
| Name | P/N | Old buffer | New buffer |
| 18119 | Negative | 231 | 316 |
| 18051 | Negative | 240 | 617 |
| 18546 | Positive | 1433 | 1802 |
| 18456 | Positive | 1038 | 1246 |

### EXAMPLE 19

### Assay Optimization—Solvent

Various solvents were tested in order to optimize substrate solubility and stability. Solvents tested include: Isopropanol, DMSO, Acetonitril, Ethylene glycol, Dioxan, 1,2-Propanediol, and 1,3-Propanediol. All the solvents tested were prepared at 0-10% concentrations in 2× reaction buffer and the final concentrations tested were 0-5%. Isopropanol, DMSO, Acetonitrile, Ethylene glycol and Dioxan were added to 3× reaction buffer to make a final 2× reaction buffer with the appropriate solvent concentration. Camb 2 was added to the final 2× buffer. For 1,2-Propanediol and 1,3-Propanediol, Camb 2 was first dissolved in the solvent at the appropriate concentration. The solvent+camb2 was then added to 3× reaction buffer to make a final 2× reaction buffer+2 uM camb2. All the experiments were performed in the TECAN Polarstar n=1 in triplicates.

### Results

Isopropanol:

TABLE 39
Experiment 1

| | Reaction rate | | |
|---|---|---|---|
| Solvents (Final | Average Blank | Average *P.C | |
| concentration) | (RFU/min) | (RFU/min) | % Loss |
| [symbol] | [symbol] | 1176.86 | — |
| Isopropanol | −49.64 | 641.57 | 45% |
| (1%) | | | |
| Isopropanol | 396.56 | 1143.93 | 3% |
| (2.5%) | | | |
| Isopropanol | 8.29 | −2.7 | 100% |
| (4%) | | | |
| Isopropanol | −1.61 | −4.19 | 100% |
| (5%) | | | |

[symbol]

indicates data missing or illegible when filed

TABLE 40
Experiment 2

| | Reaction rate | | |
|---|---|---|---|
| Solvents (Final | Average Blank | Average P.C | |
| concentration) | (RFU/min) | (RFU/min) | % Loss |
| Na₂SO₄ | −166.85 | 1601 | — |
| Isopropanol | 164.9 | 1047.42 | 35% |
| (1%) | | | |
| Isopropanol | 255.6 | 806 | 50% |
| (2.5%) | | | |
| Isopropanol | 326.53 | 676.0 | 42% |
| (4%) | | | |
| Isopropanol | 26.96 | 394.82 | 75% |
| (5%) | | | |

At all isopropanol concentrations tested, a loss in reaction rate was observed. In addition, at the high concentrations tested (4 and 5% final), salt precipitation formed. Therefore, it was concluded that this solvent is inappropriate to be used in the EV assay.

### DMSO:

TABLE 41

| | Reaction rate | | |
|---|---|---|---|
| Solvents (Final | Average Blank | Average P.C | |
| concentration) | (RFU/min) | (RFU/min) | % Loss |
| Na₂SO₄ | −83.92 | 1213.13 | — |
| DMSO (1%) | −41.59 | 1270 | — |
| DMSO (2.5%) | −1021 | 1139.26 | 7% |
| DMSO (4%) | −418.5 | 1288 | — |

At all DMSO concentrations tested (1-5%), no loss in reaction rate was observed. However, no improvement in blank stability was observed. DMSO showed no significant effect over any of the parameters tested. Therefore, it was concluded that this solvent may be used in the EV assay.

### Acetonitrile:

TABLE 42
Experiment 1

| | Reaction rate | | |
|---|---|---|---|
| Solvents (Final | Average Blank | Average P.C | |
| concentration) | (RFU/min) | (RFU/min) | % Loss |
| Na₂SO₄ | −900.8 | 1931.5 | — |
| Acetonitrile | −26.36 | 1106.6 | 43% |
| (1%) | | | |
| Acetonitrile | −11.72 | 846.73 | 57% |
| (2.5%) | | | |
| Acetonitrile | 125.03 | 706.1 | 65% |
| (4%) | | | |
| Acetonitrile | −238.6 | 308.48 | 84% |
| (5%) | | | |

TABLE 43
Experiment 2

| | Reaction rate | | |
|---|---|---|---|
| Solvents (Final | Average Blank | Average P.C | |
| concentration) | (RFU/min) | (RFU/min) | % Loss |
| Na₂SO₄ | −772.38 | 1341 | — |
| Acetonitrile | −367.4 | 1257.5 | 7% |
| (0.5%) | | | |
| Acetonitrile | −234.6 | 1002.48 | 25% |
| (1%) | | | |

At all Acetonitrile concentrations tested (1-5%), a loss in reaction rate was observed. However, an improvement in blank stability was observed (FIGS. 26A and 26B). Acetonitrile concentration was decreased to the point of minimal reaction rate inhibition (˜7% loss), but at this concentration, the contribution to blank stability was insufficient.

### Dioxan:

TABLE 44
Experiment 1

| | Reaction rate | | |
|---|---|---|---|
| Solvents (Final | Average Blank | Average P.C | |
| concentration) | (RFU/min) | (RFU/min) | % Loss |
| Na₂SO₄ | −1043.4 | 600 | — |
| Dioxan (1%) | −1331 | 1496.23 | — |
| Dioxan (2.5%) | −2279.17 | 725.55 | — |
| Dioxan (4%) | −1954.83 | 910 | — |
| Dioxan (5%) | −1907.7 | 1564.1 | — |

TABLE 45
Experiment 2

| | Reaction rate | | |
|---|---|---|---|
| Solvents (Final | Average Blank | Average P.C | |
| concentration) | (RFU/min) | (RFU/min) | % Loss |
| Na₂SO₄ | −595.26 | 1819 | — |
| Dioxan (1%) | −149.65 | 1515.03 | 17% |
| Dioxan (2.5%) | 308.5 | 934.72 | 50% |
| Dioxan (4%) | 143.45 | 585.18 | 70% |
| Dioxan (5%) | 112.32 | −127.27 | 100% |

TABLE 46
Experiment 3

| | Reaction rate | | |
|---|---|---|---|
| Solvents (Final | Average Blank | Average P.C | |
| concentration) | (RFU/min) | (RFU/min) | % Loss |
| Na₂SO₄ | 8.6 | 1834 | — |
| Dioxan (1%) | −277.7 | 1097 | 40% |
| Dioxan (2.5%) | −74.73 | 766.16 | 60% |
| Dioxan (4%) | −354.8 | 100.63 | 99% |
| Dioxan (5%) | 54.79 | 40.37 | 100% |

Dioxan solvent inhibited the enzyme activity even at the lowest concentration tested.

1,3-Propanediol with COX enzyme (1:10):

TABLE 47
GalaxyFLUOstar - Experiments 1 and 2

| | Reaction rate | | |
|---|---|---|---|
| Solvents (Final | Average Blank | Average P.C | |
| concentration) | (RFU/min) | (RFU/min) | % Loss |
| Na₂SO₄ | 172.42 | 3415.28 | — |
| 1,3-propanediol | 109.5 | 3948.19 | — |
| (1%) | | | |
| 1,3-propanediol | 240.12 | 3072.45 | 10% |
| (5%) | | | |
| | Reaction rate | | |
| Solvents (Final | Average Blank | Average P.C | |
| concentration) | (RFU/min) | (RFU/min) | % Loss |
| Na₂SO₄ | 344.96 | 5044.92 | — |
| 1,3-propanediol | 111.74 | 4125.89 | 20% |
| (1%) | | | |
| 1,3-propanediol | 232.33 | 4491.13 | 11% |
| (2.5%) | | | |
| 1,3-propanediol | 178.89 | 2956.26 | 42% |
| (4%) | | | |
| 1,3-propanediol | 212.8 | 2389.47 | 47% |
| (5%) | | | |

TABLE 48
TECAN with nonbiding plate

| | Reaction rate | | |
|---|---|---|---|
| Solvents (Final | Average Blank | Average P.C | |
| concentration) | (RFU/min) | (RFU/min) | % Loss |
| Na₂SO₄ | 358.51 | 7324.46 | — |
| 1,3-propanediol | 209.04 | 7620.53 | — |
| (1%) | | | |
| 1,3-propanediol | 41.2 | 4681.05 | 36% |
| (2.5%) | | | |
| 1,3-propanediol | 8.42 | 7238.7 | 2% |
| (4%) | | | |
| 1,3-propanediol | 47.08 | 5929.86 | 20% |
| (5%) | | | |

The results with 1,3-propanediol show high variability in the reaction rates between the three experiments. A reduction in the blank value was also observed.

1,2-Propanediol with COX enzyme (1:10):

TABLE 49
TECAN with nonbinding microplate

| | Reaction rate | | |
|---|---|---|---|
| Solvents (Final | Average Blank | Average P.C | |
| concentration) | (RFU/min) | (RFU/min) | % Loss |
| Na₂SO₄ | 178.7 | 8040 | — |
| 1,2-propanediol | 102.67 | 8588.13 | — |
| (1%) | | | |
| 1,2-propanediol | 98.11 | 6521.1 | 20% |
| (2.5%) | | | |
| 1,2-propanediol | 107.5 | 5013.13 | 30% |
| (4%) | | | |
| 1,2-propanediol | 261.74 | 4509 | 44% |
| (5%) | | | |

TABLE 50
Galaxy FLUOstar with nonbinding microplate

| | Reaction rate | | |
|---|---|---|---|
| Solvents (Final | Average Blank | Average P.C | |
| concentration) | (RFU/min) | (RFU/min) | % Loss |
| Na₂SO₄ | 275 | 6198 | — |
| 1,2-propanediol | 227.6 | 5689.6 | 9% |
| (1%) | | | |
| 1,2-propanediol | 400 | 4838.97 | 22% |
| (2.5%) | | | |
| 1,2-propanediol | 387.69 | 4291.04 | 30% |
| (4%) | | | |
| 1,2-propanediol | 459.84 | 3278.19 | 47% |
| (5%) | | | |

Except for 1% 1,2-propanediol, all concentrations tested inhibited enzymatic activity.

### Solvents Summary

Except for DMSO, Ethylene glycol and 1,3-propanediol, the solvents tested were not optimal for the EV assay.

### EXAMPLE 20

### Assay Optimization—Reaction Conditions

The following reaction conditions were examined to optimize the assay:
a. Reducing substrate concentration and increasing measurement time.
b. Optimizing Na₂SO₄ concentration.
c. Dissolving the substrate in deionized water (ddw) vs. DMSO.

### Reducing Substrate Concentration and Increasing Measurement Time

The effect of reduction in substrate (camb2) concentration and increase in measurement time was tested. The experiment was performed at a final substrate (camb2.5) concentrations of 1, 0.5, 0.25 and 0.125 μM and data analysis was made at different time intervals: 1.5-5, 5-12 and 5-22 min (FIGS. 27A-C).

A linear correlation between reaction rate and substrate concentration was demonstrated (slope=2100 and 2800 RFU/min/uM for 1.5-5 and 5-12 min respectively). When measurement time interval was changed from 1.5-5 to 5-12 min, a reduction in blank values and a modest increase in positive control values was observed (FIG. 28A). Thus, the ratio between blank and positive control increased from 3.2 to 4.8 (at 0.25 uM camb2.5), respectively (FIG. 28B). Moreover, changing the measurement time interval to 5-22 min increased the ratio to 6 (at 0.25 uM camb2.5, FIG. 28B). FIG. 28B also shows similar ratios between blank and positive control at different substrate concentrations. FIG. 28C shows an improvement in CV values in both positive control and blank, when measurement time interval changed from 1.5-5 to 5-12 min. According to these results, a reduction in blank values and increase in the stability of blank values were achieved. Based on these results, substrate concentration was reduced to a final concentration of 0.25 uM and results will be analyzed between 5-10 min.

### Optimizing Na₂SO₄ Concentration

Sodium sulfate generally has an ambivalent effect: it increase 3C protease activity and reduces substrate solubility. Once it was decided that the substrate concentration would be reduced to 0.25 uM and results analyzed at between 5-10 min, the Na₂SO₄ concentration was optimized. The sodium sulfate final concentration was lowered as much as possible without affecting assay activity, while improving solubility. 0.6-0.4M Na₂SO₄ was tested under the new assay conditions.

TABLE 51

| | | Positive | | Positive | | Positive |
|---|---|---|---|---|---|---|
| | Blank | control | Blank | control | Blank | control |
| | 0.6M Na2SO4 | | 0.5M Na2SO4 | | 0.4M Na2SO4 | |
| | (RFU/min) | | (RFU/min) | | (RFU/min) | |
| EXP-1 | 92 | 862 | 237 | 791 | 176 | 929 |
| EXP-2 | 177 | 890 | 143 | 992 | 146 | 402 |
| EXP-3 | 121 | 824 | 111 | 829 | 36 | 404 |
| | 0.6M Na2SO4 | | 0.5M Na2SO4 | | 0.4M Na2SO4 | |
| EXP-1 | 154 | 679 | 95 | 697 | 102 | 362 |
| EXP-2 | 171 | 918 | 181 | 665 | 118 | 536 |
| EXP-3 | 90 | 921 | 64 | 620 | 89 | 382 |
| EXP-1 | 130 | 739 | 103 | 432 | 72 | 312 |
| EXP-2 | 89 | 770 | 30 | 601 | 17 | 228 |
| Averages ± SE | | 825 ± 31 | | 703 ± 56 | | 444 ± 75 |

Data analysis was performed between 5-10 min.
The assay activity was measured in triplicates by three scientists. The results in Table 51 are the average of the three triplicates.

According to the results, reduction in sodium sulfate concentration reduced the enzymatic activity from 825 at 0.6M, to 700 at 0.5M, and 444 at 0.4M. Based on these results, it was decided not to change the sodium sulfate concentration and continue to work with 0.6M sodium sulfate.

Dissolving the Substrate in Deionized Water (ddw) vs. DMSO.

To examine the effect of substrate dilution in DMSO vs. DDW under the new assay conditions, substrate stock (1 mM in DMSO) was diluted 1:8 in DMSO or in DDW to yield the working stock. The working stocks were further diluted 1:250 in 2× RB for a concentration of 0.5 uM (0.25 uM final in the well).

The assay activity was measured in triplicates by three scientists. The results in Table 52 are the average of the three triplicates.

TABLE 52

| | DMSO | | DDW | |
|---|---|---|---|---|
| | Blank | Positive control | Blank | Positive |
| | (RFU/min) | (RFU/min) | (RFU/min) | (RFU/min) |
| | 169 ± 15 | 1023 ± 36 | 168 ± 27 | 747 ± 68 |
| | 228 ± 51 | 987 ± 126 | 159 ± 18 | 832 ± 64 |
| Average | 198.5 | 1005 | 163.5 | 789.5 |
| stdev | 41.7193 | 25.45584412 | 6.363961031 | 60.10408 |
| CV | 21.01728 | 2.532919813 | 3.892330906 | 7.612929 |
| SE | 29.5 | 18 | 4.5 | 42.5 |

Data Analysis was Performed Between 5-10 Min.

The blank slope was the same in both DMSO and DDW in the first repetition. In the second repetition, the DDW stock showed a reduced blank slope; however, that value was insignificant over the standard deviation. Moreover, the results show that the reaction rate was 25% lower when the substrate was diluted with DDW compared to DMSO. Based on these results and the fact that Camb2 is expected to be more stable in DMSO than in DDW, DMSO was chosen as a diluent for the substrate.

### Validation Tests

Two positive and two negative CSF samples were tested under the new assay conditions.

TABLE 53

| Sample's | | New conditions |
|---|---|---|
| Name | P/N | (RFU/min) |
| Blank | | 41 |
| Positive | | 362 |
| control | | |
| 710022 | Negative | 29 |
| 18911 | Negative | 15 |
| 18546 | Positive | 690 |
| 19055 | Positive | 870 |

These results indicate that the samples tested performed adequately in the new assay conditions. The negative samples (710022 and 18911) showed no reaction rate above the blank. Positive samples had a high reaction rate, higher than the positive control reaction.

### Conclusions:

The assay conditions were changed to 25% Camb2 substrate concentration and kinetic measurement time at 5-10 mM. Na₂SO₄ concentration remained at 0.6M. These assay conditions were validated with clinical samples.

### EXAMPLE 21

### Analysis of Clinical Samples for Enterovirus After Partial Assay Optimization

Before optimizing the enterovirus assay, the analysis comprised analyzing CSF samples (whole, frozen) with the addition of the substrate to a 2× reaction buffer (2× RB) with a final concentration of 0.9M Na₂SO₄ and an inhibitor cocktail (I8) that was mostly directed to reducing WBC background. That assay yielded 80% and 81% sensitivity and specificity, respectively. See Example 16. The following changes were made to the assay:
The final concentration of Na₂SO₄ in the reaction buffer was lowered to 0.6M.
The substrate concentration was reduced to 25% and is added to the 2× reaction buffer.
Calculation time was changed to 5-10 min. Gain was raised to 100.
The inhibitor cocktail was replaced with I10 with 2.5 minutes incubation prior to the substrate addition
Non binding plates and tubes were used.
   CSF samples were analyzed using the following conditions:

### Reagents and Instruments:

Inhibitor cocktail—I10
HEPES Buffer: HEPES 25 mM
2× reaction buffer: 50 mM HEPES, 10 mM EDTA, 10 mM EGTA, 1.2M Na2SO4, sodium azide 0.1%, pH 8.5
Artificial CSF: NaCl 125 mM, KCl 2.5 mM, MgCl 1 mM, NaH₂PO₄ 1.25 mM, CaCl₂ 2 mM, Glucose 2.5 mM, NaHCO₃ 25 mM, 0.1% Sodium azide, HEPES 25 mM, pH 7.45.
Enzyme: Recombinant enterovirus protease from strain “Echo”diluted 1:18 in dilution buffer
Substrate: Camb2.5 (1 mM) diluted 1:8 in DMSO
Fluorimeter: BMG, Polar-Star Galaxy
   Ex: 520/10 nm, Em: 570/20 nm.
   Temperature: RT.
   Total reading Time: 10 min.
   Gain=100
      Reaction plate: Non binding black bottom plate (catalog #—655900, Greiner)

### Assay Procedure:

1. Thaw samples at room temperature for 30 min in a biological hood.
2. Prepare assay layout according to the number of samples to be run in the BMG fluorimeter and the well composition shown in Table 54

TABLE 54

| | Negative | Positive | |
|---|---|---|---|
| Test tube/Item | Control | control | Sample |
| Artificial CSF/Sample | 50 | 45 | 50 |
| Echo batch II (1:18) | — | 5 | — |
| Inhib. mix | 5 | 5 | 5 |
| RB 2X + substrate | 50 | 50 | 50 |
| Total volume | 105 | 105 | 105 |

3. Prepare sufficient amount of inhibitor cocktail I10 (150 ul for 24 wells).
4. Add artificial CSF/sample to the wells (black plate NB cat. 655900).
5. Add Echo protease to p.c. (positive control) wells.
6. Dilute Camb2.5 (0.125 mM) 1:250 in 2× RB.
7. Add inhibitor cocktail to all wells.
8. Add 50 ul 2× RB+substrate to all the wells.
9. Cover the wells with a sealer.
10. Immediately start measurement in fluorimeter.
   Samples: The samples are clinical samples that were collected from patients suspected of having meningitis as described in Example 16 and frozen as a “whole” sample.

TABLE 55

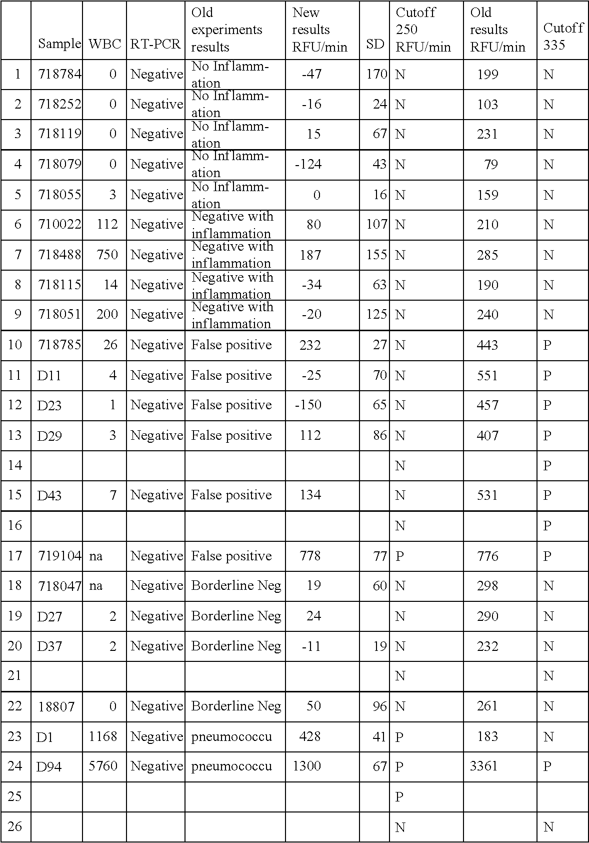

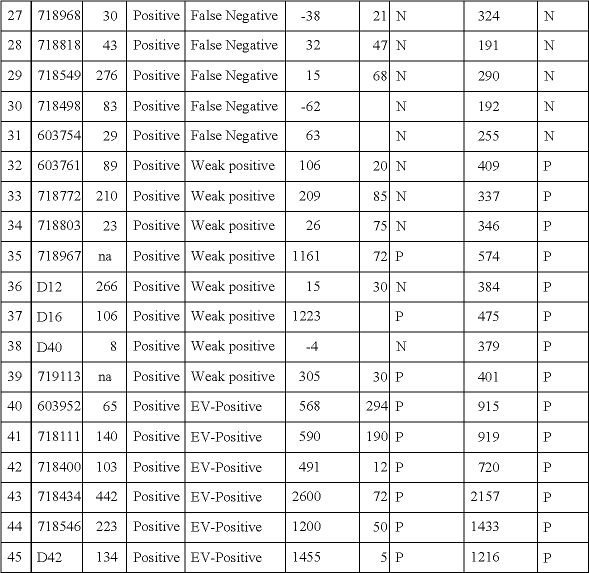

The cutoff value was set at 250 RFU/min. As shown in Table 55, all negative (1-9) and positive (40-45) results from old experiments retained their correlation to the RT-PCR assay under the new assay conditions. The biggest improvement observed was in the false positive group (10-17). Of these 8 old false positive results, only one remained false positive (17) and the other 7 tested negative under the new assay conditions. Furthermore, of these samples, two were hemolytic samples (14 and 16). This suggests that the new assay conditions can cope better with hemolytic samples. Another interesting finding was observed in the borderline negative group (18-22). Four out these five samples tested significantly lower than the cutoff value under the new assay conditions. The one sample that remained slightly below the cutoff was a hemolytic sample. The improvements in the false positive and borderline negative groups can be attributed to the improvements made to the blank slope, making the system more robust.

On the other hand, the effect of the new conditions was observed in the weak positive group (32-39). Out of the eight samples that previously tested weakly positive, five tested negative under the new assay conditions. However, two samples that previously tested weakly positive (35 and 37) tested strongly positive under the new assay conditions. One sample (39) remained weakly positive. These findings suggest that the majority of samples with reaction rates slightly above the cutoff were artifacts due to the lack of robustness under the old assay conditions. No change in the false negative group (26-31) was observed.

Moreover, positive signals were still obtained from the three bacterial samples tested (23-25). Though the assay was carried out with inhibitors specifically targeted against the bacteria in question (phosphoramidon for Pneumococcus (23 and 24) and 2,6 pyridine for Meningococcus (25)), they still could not abolish the unspecific background activity that resulted.

As shown in Table 56, the new assay conditions were able to improve specificity, mainly by reducing past false positive results (10-16) that exhibited reaction rates slightly above the cutoff. This improvement can be attributed to the increased robustness of the blank under the new assay conditions. The majority of false positive samples were bacterial (3 out of 4). However, sensitivity was reduced from 70% to 45% under the new assay conditions. This reduction originated solely from previously tested weak samples (32-34, 36 and 38). This reduction can also be attributed to the increased robustness of the blank under the new assay conditions, eliminating the positive artifacts resulting from the old assay conditions.

TABLE 56

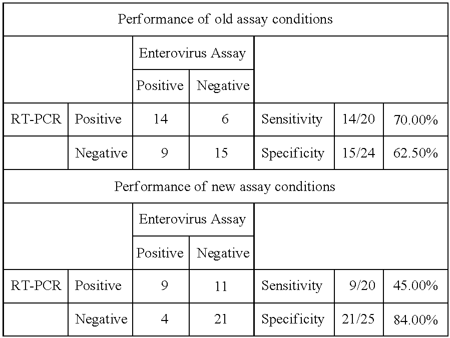

1-139. (canceled)

140. A method of detecting the presence or absence of a biomarker of a pathogen, disease, dysfunction of a biological cascade or medical condition in a subject by detecting the presence or absence of an endogenous substrate in a biological sample comprising:
a. providing the biological sample from the subject that may or may not contain the endogenous substrate;
b. providing one or more test reactions by contacting the biological sample with an enzyme indicative of the biomarker of a pathogen, disease, dysfunction of a biological cascade or medical condition and a substrate comprising a signaling moiety; wherein the enzyme modifies the endogenous substrate and the substrate comprising the signaling moiety, and wherein modification of the substrate comprising, the signaling moiety by the enzyme produces a signal from the signaling moiety;
c. providing data from a control reaction comprising the enzyme and the substrate comprising the signaling moiety;
d. detecting the signal produced by the signaling moiety in the test reaction; and
e. wherein the presence or quantity of the biomarker of the pathogen, disease, dysfunction of a biological cascade or medical condition is indicated by a difference caused by the presence of the endogenous substrate in the biological sample between the signal produced in the test reaction and the data from the control reaction.

141. The method of claim 140, wherein the enzyme modifies the substrate comprising the signaling moiety by cleaving the substrate comprising the signaling moiety.

142. The method of claim 140, wherein the signaling moiety is a signaling moiety chosen from an enzyme, a fluorophore, a chromophore, a protein, a peptide, a chemiluminescent substance, a quencher, a Fluorescence Resonance Energy Transfer (FRET) pair, a pre-enzyme, a lectin, an aptamer and a radiosotope.

143. The method of claim 143, wherein the substrate comprising the signaling moiety further comprises a separation moiety.

144. The method of claim 143, wherein the separation moiety is a separation moiety chosen from an immunological binding agent, a magnetic binding moiety, a peptide binding moiety, an affinity binding moiety, a lectin, an aptamer and a nucleic acid moiety.

145. The method of claim 143, further comprising separating, modified substrate comprising the signaling moiety from unmodified substrate comprising the signaling moiety.

146. The method of claim 140, wherein the pathogen, disease, biological cascade or medical condition is selected from a group consisting of a bacterial infection, methicillin resistant Staphylococcus aureus infection, an autoimmune disorder, genetic disorder, coagulation disorder, cancer, inflammation, neurodegenerative disorder, hypertension, vasodilation, diabetes, allergy, a coagulation cascade, fibrinolysis cascade, kinin cascade, signaling cascade, mitogen-activated protein kinase (MAPK) cascade, and inflammation cascade or wherein the endogenous substrate is selected from a group consisting of procalcitonin and phenol soluble modulin.

147. The method of claim 140, further comprising contacting the biological sample with one or more agents selected from a group consisting of inhibitors of non-specific enzymatic activity and activators of the enzyme indicative of the biomarker of a pathogen, disease, dysfunction of a biological cascade or medical condition.

148. The method of claim 140, wherein the substrate comprising the signaling moiety is fused to a second enzyme that becomes activated upon cleavage of the substrate comprising the signaling moiety by the enzyme indicative of the biomarker of a pathogen, disease, or medical condition, and the activated second enzyme modifies a second substrate comprising a second signaling moiety and produces a signal from the signaling moieties.

149. A kit comprising:
a. an enzyme that modifies an endogenous substrate in a biological sample and a substrate comprising a signaling moiety;
b. the substrate comprising the signaling moiety;
c. and an instructions for performing a method of detecting the presence or absence of a biomarker of a pathogen, disease, or medical condition in a subject by detecting the presence or absence of an endogenous substrate in a biological sample comprising:
i. providing the biological sample from the subject that may or may not contain the endogenous substrate;
ii. providing a test reaction by contacting the biological sample with an enzyme indicative, of the biomarker of a pathogen, disease, or medical condition and a substrate comprising a signaling moiety; wherein the enzyme modifies the endogenous substrate and the substrate comprising the signaling moiety, and wherein modification of the substrate comprising the signaling moiety by the enzyme produces a signal from the signaling moiety;
iii. providing data from a control reaction comprising the enzyme and the substrate comprising the signaling moiety;
iv. detecting the signal produced by the signaling moiety in the test reaction; and
v. wherein the presence or quantity of the biomarker of the pathogen, disease, or medical condition is indicated by a difference caused by the presence of the endogenous substrate in the biological sample between the signal produced in the test reaction and the data from the control reaction.

150. The kit of claim 149, further comprising one or more agents selected from a group consisting of inhibitors of enzymatic activity and activators of the enzyme.

151. A method for detecting the presence or absence of enzymatic activity in a biological sample by detecting the presence or absence of an enzyme comprising:
a. contacting said biological sample that may or may not contain the enzyme obtained from a subject with a substrate comprising, a signaling moiety; wherein the enzyme modifies the substrate and wherein modification of the substrate produces a signal from the signaling moiety; and
b. detecting a signal produced from the signaling moiety; wherein the signal produced is indicative of enzymatic activity in the sample, further wherein the enzymatic activity is indicative of a pathogen, disease, or medical condition in a subject.

152. The method of claim 151, wherein the method further comprises contacting the biological sample with one or more agents chosen from inhibitors of enzymatic activity or with one or more activators of the enzyme.

153. The method of claims 151, wherein the signaling moiety is a signaling moiety chosen from an enzyme, a fluorophore, a chromophore, a protein, a peptide, a chemiluminescent substance, a quencher, a fluorescence Resonance Energy Transfer (FRET) pair, a pre-enzyme, a lectin, an aptamer, and a radiosotope.

154. The method of claim 151, wherein the substrate further comprises a separation moiety.

155. The method of claim 154, wherein the separation moiety is a separation moiety chosen from an immunological binding agent, a magnetic binding moiety, a peptide binding moiety, an affinity binding moiety, a lectin, an aptamer, and a nucleic acid moiety.

156. The method of claim 154, further comprising separating modified substrate from unmodified substrate.

157. The method of claim 151, wherein the biological sample is chosen from white blood cells (WBC), cerebrospinal fluid (CSF) and serum.

158. The method of claim 151, wherein the enzyme is selected from a group consisting of a cytomegalovirus (CMV) protease, a human immunodeficiency virus (HIV) protease, a human T-cell lymphotrophic virus (HTLV) virus protease, a secreted aspartyl proteinase (Sap), Sap2, phospholipase B, lipase, bacterial neuraminidase, aromatase and prostate specific antigen (PSA).

159. The method of claim 151, wherein the substrate is selected from a group consisting of a substrate comprising the amino acid sequence chosen from VXAA/S (SEQ ID NO.: 23), G-V-V-N-A-/S-C-R (SEQ ID NO.: 19); S-Y-V-L-A-/S-V-S (SEQ ID NO.: 20); N-N-V-E-A-/A-T-S (SEQ ID NO.: 21); T-A-V-N-A-/S-G-N (SEQ ID NO.: 22); R-G-V-V-N-A-/-S-S-R-L-A (SEQ ID NO.: 26), R-G-V-V-N-A/-S-S-R-L-A-C (SEQ ID NO.: 27), (S/G)(Q/G/R/K)(N/C/D)(Y/hydrophobic/aromatic)-/-P(W/hydrophobic)(V/Q) (SEQ ID NO.: 28), (V/L/T/P)X(hydrophobic)(F/L)-/-V(hydrophobic)Q (SEQ ID NO.: 29); KVKV(F/L)-/-VVQPK (SEQ ID NO.: 30); PPX(hydrophobic)L-/-PI (SEQ ID NO.: 31), R-L-I-Q-K-R-S-D (SEQ ID NO.: 3); L-I-Q-K-R-S-D-V (SEQ ID NO.: 4); F-V-N-Q-H-L (SEQ ID NO.: 5); L-V-E-A-L-Y-L-V (SEQ ID NO.: 6); E-A-L-Y-L-V-C-G (SEQ ID NO.: 7); E-R-G-F-F-Y-T-P (SEQ ID NO.: 8); P-A-L-F-F-R-L (SEQ ID NO.: 9); L-V-I-H-T (SEQ ID NO.: 10); H-Q-V-Y-F-V-R-K (SEQ ID NO.: 11); P-A-R-F-F-R-L (SEQ ID NO.: 12); P-A-E-F-F-A-L (SEQ ID NO.: 13); P-L-E-M-F-A-L (SEQ ID NO.: 14), HSSKLQ (SEQ ID NO.: 15); QFYSSN (SEQ ID NO.: 16); GAGLRLSSYY-SGAG (SEQ ID NO.: 17) and SSIYSQTEEQ (SEQ ID NO.: 1.8); and methoxy-4-trifluoromethyl-coumarin (MFC).

160. The method of claim 151, wherein said pathogen, disease, or medical condition is chosen from a fungal infection, a meningitis infection, dysfunctional endocrine system and prostate cancer.

161. The method of claim 160, wherein the fungus is chosen from Candida, Cryptococcus neoformans, Aspergillus fumigates, Blastocladiomycota, chytridiomycota, Dikarya, Glomeromycota, Microsporidia, and Neocallimastigomycota.

162. The method of claim 151, wherein the substrate comprising the signaling moiety comprises a sialic acid and a carbohydrate linked by a linker chosen from an α2-3, α2-6, α2-8, α2-9 and cyclic neuraminidic acid linkages. The method of claim 14, wherein the one or more inhibitors of enzymatic activity is one or more inhibitors chosen from an inhibitor of granzyme H, proteinase 3, dipeptidyl peptidase, PrSSII, HtrA2, Calpain 7, Kallikrein 12, Cathepsin A, Cathepsin H, calpain-11, α-1-Proteinase inhibitor, Diprotin A, L-2,4-Diaminobutyryl-piperidinamide HtrAl inhibitor, Ucf-101, 3-(4-Iodophenyl)-2-mercapto-(Z)-2-propenoic acid, HAl-2A, Ecotin, Ebelactone B, chymostatin, human stefin A, human stefin B, calpastatin and inhibitors inhibit proteases from white blood cells, pneumococcus protease activity, meningococcus protease activity or a combination thereof, inhibitors that inhibit enzymatic activity associated with bacterial meningitis infection but does not inhibit viral meningitis enzymatic activity, cocktail of inhibitors chosen from: a) cocktail II 9a: 4-(2-Aminoethyl)benzenesulfonyl fluoride hydrochloride (AEBSF), aprotinin, bestatin, trans-Epoxysucciny-L-leucyl-amido(4-guanidine)butane (E-64), Eglin C, (3S,3aS,6aR)-3-Isopropyl-l-(methanesulfonyl)-4-[4-(l-piperidinyl)-2(E)-butenoyl]perhydropyrrolo[3,2b]pyrrol-2(lH)-one hydrochloride (GW311616A), pepstatin A, heparin, and phosphoramidon; b) cocktail II 9b: 4-(2-Aminoethyl)benzenesulfonyl fluoride hydrochloride (AEBSF), aprotinin, bestatin, trans-Epoxysucciny-L-leucyl-amido(4-guanidino)butane (E-64), Eglin C, (3S,3aS,6aR)-3-Isopropyl-l-(methanesulfonyl)-4-[4-(l-piperidinyl)-2(E)-butenoyl]perhydropyrrolo[3,2b]pyrrol-2(lH)-one hydrochloride (GW311616A), pepstatin A, heparin, and 2-6-pyridinedicarboxylic acid; and c) cocktail 110: 4-(2-Aminoethyl)benzenesulfonyl fluoride hydrochloride (AEBSF), aprotinin, bestatin, trans-Epoxysucciny-L-leucyl-amido(4-guanidine)butane (E-64), Eglin C, (3S,3aS,6aR)-3-isopropyl-l-(methanesulfonyl)-4-[4-(l-piperidinyl)-2(E)-butenoyl]perhydropyrrolo[3,2b]pyrrol-2(lH)-one hydrochloride (GW311616A), pepstatin A, heparin, phosphoramidon, and 2-6-pyridinedicarboxylic acid; optionally wherein the activator is Na₂SO₄.

163. The method of claim 161, wherein the meningitis is viral meningitis if a signal is produced in the presence of one or more inhibitors that inhibit enzymatic activity associated with bacterial meningitis infection but do not inhibit viral meningitis enzymatic activity, or wherein the subject has a meningitis infection if a signal is reduced or not produced in the presence of one or more inhibitors that inhibit meningitis enzymatic activity, compared to a signal produced in the absence of these inhibitors.
